SEQUENCE LISTING (14 sequences)

SEQ ID NO: 1 - protein, 234 aa, Homo sapiens
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFNIVILLAGGFLSKSLVFSVLFAVTLRSFVP

SEQ ID NO: 2 - protein, 150 aa, Homo sapiens
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFRCSTLSFSWLVDS

SEQ ID NO: 3 - protein, 225 aa, Homo sapiens
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRYSFQVPGPLVWTLSPLFPSLCAERM

SEQ ID NO: 4 - protein, 250 aa, Homo sapiens
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIREKSMTFGIRRLDVESHPLPPLHTGHFRISQFFSQAGTQSLHSCYKGKPTP

SEQ ID NO: 5 - protein, 185 aa, Homo sapiens
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFN

SEQ ID NO: 6 - protein, 175 aa, Homo sapiens
LKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFN

SEQ ID NO: 7 - protein, 107 aa, Homo sapiens
LKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGF

SEQ ID NO: 8 - DNA, 18 bp, Artificial Sequence
  Primer
gtggtgaccaaggggttc

SEQ ID NO: 9 - DNA, 20 bp, Artificial Sequence
  Primer
agatggatgtggctggaagt

SEQ ID NO: 10 - DNA, 20 bp, Artificial Sequence
  Primer
gtgaccaagggtttttcagg

SEQ ID NO: 11 - DNA, 20 bp, Artificial Sequence
  Primer
acaccggaaccctgatgata

SEQ ID NO: 12 - DNA, 19 bp, Artificial Sequence
  Primer
aaaggcaagaacgcctgac

SEQ ID NO: 13 - DNA, 20 bp, Artificial Sequence
  Primer
gggactttaccaagagggac

SEQ ID NO: 14 - protein, 1273 aa, SARS coronavirus
  S protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

(19) United States
(12) Patent Application Publication
(10) Pub. No.: US 2023/0273208 A1
(43) Pub. Date: Aug. 31, 2023

(54) SOLUBLE TREM-1 AS A MARKER OF SEVERITY OR COMPLICATIONS FOR A SUBJECT SUFFERING FROM A CORONAVIRUS INFECTION

(71) Applicants: INOTREM, Vandoeuvre-lès-Nancy (FR); CENTRE HOSPITALIER REGIONAL UNIVERSITAIRE DE NANCY, Nancy Cedex (FR); UNIVERSITE DE LORRAINE, Nancy Cedex (FR); CENTRE NATIONAL DE LA RECHERCHE SCIENTIFIQUE, Paris (FR); STICHTING RADBOUD UNIVERSITAIR MEDISCH CENTRUM, Nijmegen (NL)

(72) Inventors: Jean-Jacques GARAUD, Paris (FR); Marc DERIVE, Villers-lès-Nancy (FR); Margarita SALCEDO-MAGGUILLI, Châtillon (FR); Lucie JOLLY, Nancy (FR); Sébastien GIBOT, Réméréville (FR); David MOULIN, Vandoeuvre-lès-Nancy (FR); Mihai NETEA, Nijmegen (NL); Aline DE NOOIJER, Nijmegen (NL)

(73) Assignees: INOTREM, Vandoeuvre-lès-Nancy (FR); CENTRE HOSPITALIER REGIONAL UNIVERSITAIRE DE NANCY, Nancy Cedex (FR); UNIVERSITE DE LORRAINE, Nancy Cedex (FR); CENTRE NATIONAL DE LA RECHERCHE SCIENTIFIQUE, Paris (FR); STICHTING RADBOUD UNIVERSITAIR MEDISCH CENTRUM, Nijmegen (NL)

(21) Appl. No.: 18/007,849

(22) PCT Filed: Jun. 7, 2021
(86) PCT No.: PCT/EP2021/065196

§ 371 Date: Dec. 2, 2022

(30) Foreign Application Priority Data
Jun. 5, 2020 (EP) 20305604.9
Mar. 3, 2021 (EP) 21160546.4

(51) Int. Cl. G01N 33/569

(52) U.S. Cl. CPC G01N 33/56983; G01N 2333/70503; G01N 2469/20

(57) ABSTRACT

The use of soluble triggering receptor expressed on myeloid cells-1 (sTREM-1) as a marker in a method for identifying a subject suffering from a disease caused by a coronavirus such as COVID-19 at risk of having or developing a severe form and/or a complication or at risk of death, in a method for assessing the severity of a disease caused by a coronavirus such as COVID-19, and in a method for monitoring a subject suffering from a disease caused by a coronavirus such as COVID-19.

### FIELD OF INVENTION

The present invention relates to the identification and monitoring of a subject suffering from a disease caused by a coronavirus, in particular of a subject at risk of a severe form of the disease or at risk of complication(s). The present invention notably relates to soluble triggering receptor expressed on myeloid cells-1 (sTREM-1) as a marker of a disease caused by a coronavirus, in particular as a marker of severity and/or complication(s), for use in methods for identifying, assessing, monitoring a subject suffering from a disease caused by a coronavirus.

### BACKGROUND OF INVENTION

Coronaviruses (CoVs) are ribonucleic acid (RNA) viruses of the Coronaviridae family, notably characterized by a distinctive morphology as seen with electron microscopy, i.e., a crownlike appearance resulting from club-shaped spikes projecting from the surface of their envelope. Coronaviruses infect mammals and birds and cause a wide range of respiratory, gastrointestinal, neurologic, and systemic diseases.

Human coronaviruses were initially thought to cause only mild respiratory infections in most cases, such as the common cold. Four endemic human CoVs are thus estimated to account for 10% to 30% of upper respiratory tract infections in human adults. However, in recent years, two highly pathogenic coronaviruses causing severe respiratory diseases emerged from animal reservoirs: severe acute respiratory syndrome coronavirus (SARS-CoV) first identified in 2003 and Middle East respiratory syndrome coronavirus (MERS-CoV) first identified in 2012.

In December 2019, the Wuhan Municipal Health Committee, China, identified a new infectious respiratory disease of unknown cause. Coronavirus RNA was quickly identified in some of the patients and in January 2020, a full genomic sequence of the newly identified human coronavirus SARS-CoV-2 (previously known as 2019-nCoV) was released by Shanghai Public Health Clinical Center & School of Public Health, Fudan University, Shanghai, China. The genomic sequence of SARS-COV-2 has 82% nucleotide identity with the genomic sequence of human SARS-CoV (Chan et al., Emerg Microbes Infect. 2020; 9(1):221-236). Moreover, as previously shown for SARS-CoV, SARS-CoV-2 utilizes ACE2 (angiotensin converting enzyme 2) as receptor for viral cell entry (Hoffmann et al., Cell. 2020; 181(2):271-280.e8).

In infected subjects exhibiting symptoms, the disease caused by SARS-COV-2 is termed “coronavirus disease 2019” (COVID-19). COVID-19 is a respiratory illness with a broad clinical spectrum. The majority of affected subjects experience mild or moderate symptoms. COVID-19 generally presents first with symptoms including headache, muscle pain, fatigue, fever and respiratory symptoms (such as a dry cough, shortness of breath, and/or chest tightness). Other reported symptoms include a loss of smell and/or taste. Some subjects develop a severe form of COVID-19 that may lead to pneumonitis and acute respiratory failure. Complications of COVID-19 include thrombotic or thromboembolic complications, pulmonary embolism, cardiovascular failure, renal failure, liver failure and secondary infections. It is estimated that about 5% of subjects suffering from COVID-19 will require hospitalization, with about 15-25% of the hospitalized subjects requiring admission in intensive care unit (ICU). SARS-CoV-2 infection is thought to be asymptomatic or causing little or no clinical manifestations in 30 to 60% of infected subjects.

Global efforts to identify efficient diagnosis, prognosis and monitoring markers are ongoing. In particular, it would be helpful to be able to identify subjects affected with COVID-19 likely to develop a severe form of the disease and/or a complication, to assess the severity of COVID-19 in a subject, and to monitor subjects affected with COVID-19.

Therefore, there is still a need for markers allowing to identify, assess, and monitor subjects suffering from a disease caused by a coronavirus, such as COVID-19 caused by a SARS-CoV-2, in particular subjects likely to develop a severe form of the disease and/or a complication of the disease.

The present invention relates to soluble triggering receptor expressed on myeloid cells-1 (sTREM-1) as a marker of a disease caused by a coronavirus, such as COVID-19 caused by SARS-CoV-2, in particular as a marker used in a method for identifying a subject suffering from a disease caused by a coronavirus at risk of a severe form and/or a complication, in a method for assessing the severity of a disease caused by a coronavirus, and in a method for monitoring a subject suffering from a disease caused by a coronavirus.

### SUMMARY

A first object of the invention is an in vitro method for identifying a subject suffering from a disease caused by a coronavirus infection as being at risk of having or developing a severe form and/or a complication of the disease caused by a coronavirus infection or at risk of death occurring after the coronavirus infection, said method comprising:
measuring the level of soluble triggering receptor expressed on myeloid cells-1 (sTREM-1) in a biological sample from the subject; and
comparing the level of sTREM-1 measured in the biological sample from the subject to a reference value.

In one embodiment, the complication of the disease caused by a coronavirus infection is selected from the group consisting of respiratory failure, including acute respiratory failure or acute respiratory distress syndrome (ARDS); respiratory failure requiring oxygen therapy (including non-invasive ventilation); respiratory failure requiring mechanical ventilation; persistence of respiratory failure including the requirement for prolonged mechanical ventilation, in particular prolonged mechanical ventilation lasting more than 15 days, and failed extubation; secondary infection or superinfection; thrombotic complications (also referred to as thromboembolic complications) including venous and/or arterial thromboembolism, deep venous thrombosis, pulmonary embolism, and cerebrovascular accidents; cardiocirculatory failure (which may also be referred to as cardiovascular failure); renal failure such as acute kidney injury (AKI); liver failure; and any combinations thereof.

Another object of the invention is an in vitro method for determining the severity of a disease caused by a coronavirus infection in a subject, said method comprising:
measuring the level of soluble triggering receptor expressed on myeloid cells-1 (sTREM-1) in a biological sample from the subject; and
comparing the level of sTREM-1 measured in the biological sample from the subject to a reference value.

In one embodiment, the method for determining the severity of a disease caused by a coronavirus infection in a subject allows the monitoring over time of said in the subject, and the method comprises measuring the level of sTREM-1 in biological samples from the subject obtained on at least two occasions, preferably separated by at least 24 hours.

In one embodiment, the coronavirus is severe acute respiratory syndrome coronavirus 2 (SARS-CoV-2) and the disease caused by a coronavirus infection is coronavirus disease 2019 (COVID-19).

In one embodiment, the level of sTREM-1 is a transcription level of sTREM-1 or a translation level of sTREM-1. In one embodiment, the biological sample is a blood sample, preferably a serum sample.

In one embodiment, the subject requires hospitalization. In one embodiment, the subject requires respiratory support. In one embodiment, the level of sTREM-1 is measured at day 3 following hospitalization.

### Definitions

In the present invention, the following terms have the following meanings:

“TREM-1” refers to “triggering receptor expressed on myeloid cells-1” and is also sometimes referred to as CD354. TREM-1 is a membrane-bound glycoprotein receptor belonging to the immunoglobulin (Ig) superfamily that is notably expressed on myeloid cells. TREM-1 activates downstream signaling pathways with the help of an adapter protein called DAP12 (DNAX-activating protein of 12 kDa). TREM-1 comprises three distinct domains: an Ig-like structure (mostly responsible for ligand binding), a transmembrane part and a cytoplasmic tail which associates with DAP12. Unless specified otherwise, the TREM-1 protein has an amino acid sequence as set forth in SEQ ID NO: 1, corresponding to UniProtKB/Swiss-Prot accession number Q9NP99-1, last modified on Oct. 1, 2000 and to UniProtKB accession number Q38L15-1, last modified on Nov. 22, 2005. Several transcripts are known for TREM-1. The transcript commonly referred to as TREM1-201 (transcript ID ensembl ENST00000244709.8) encodes an amino acid sequence as set forth in SEQ ID NO: 1. The transcript commonly referred to as TREM1-202, also known as TREM-1 isoform 2 (ensembl transcript ID ENST00000334475.10) encodes an amino acid sequence as set forth in SEQ ID NO: 2 (corresponding to UniProtKB/Swiss-Prot accession number Q9NP99-2). The transcript commonly referred to as TREM1-207, also known as TREM-1 isoform 3 (ensembl transcript ID ENST00000591620.1) encodes an amino acid sequence as set forth in SEQ ID NO: 3 (corresponding to UniProtKB/Swiss-Prot accession number Q9NP99-3). The transcript commonly referred to as TREM1-204 (ensembl transcript ID ENST00000589614.5) encodes an amino acid sequence as set forth in SEQ ID NO: 4 (corresponding to UniProtKB/Swiss-Prot accession number K7EKM5-1, last modified Jan. 9, 2013).

“sTREM-1”, for “soluble triggering receptor expressed on myeloid cells-1”, refers to a soluble form of TREM-1 lacking the transmembrane and intracellular domains of TREM-1. In one embodiment, sTREM-1 thus corresponds to the soluble form of the extracellular domain of TREM-1. The soluble TREM-1 may be generated by proteolytic cleavage of TREM-1 Ig-like ectodomain from the membrane-anchored TREM-1 by matrix metalloproteinases (Gomez-Pina et al., J Immunol. 2007 Sep. 15; 179(6):4065-73). In one embodiment, sTREM-1 thus corresponds to a truncated TREM-1 shed from the membrane of myeloid cells, in particular from activated myeloid cells. It was also suggested that sTREM-1 results from an alternative splicing of TREM-1 mRNA. A TREM-1 splice variant was characterized in 2015 by Baruah et al. (J Immunol. 2015 Dec. 15; 195(12):5725-31), and was found to be secreted from primary and secondary human neutrophil granules. In one embodiment, sTREM-1 thus corresponds to a TREM-1 splice variant, in particular to the TREM-1 transcript commonly referred to as TREM1-202, also known as TREM-1 isoform 2, encoding an amino acid sequence as set forth in SEQ ID NO: 2.

“About” preceding a figure encompasses plus or minus 10%, or less, of the value of said figure. Thus, for example, “about 10” encompasses the values ranging from 9 to 11, and “about 100” encompasses the values ranging from 90 to 110. It is to be understood that the value to which the term “about” refers is itself also specifically, and preferably, disclosed.

“Biomarker” or “biological marker” refers to a variable that can be measured in a biological sample from a subject.

“Electrochemiluminescence immunoassay (ECLIA)” refers to an immunoassay wherein the detection of the signal is based on electrochemiluminescence, i.e., a form of chemiluminescence in which the light-emitting chemiluminescent reaction is preceded by an electrochemical reaction.

“Identity” or “identical”, when used in the present invention in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”) Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988). Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. \2, 387 (1984); Genetics Computer Group, University of Wisconsin, Madison, Wis.), BLASTP, BLASTN, and FASTA (Altschul et al., J. MoI. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., J. MoI. Biol. 215, 403-410 (1990)). The well-known Smith Waterman algorithm may also be used to determine identity.

“Measuring” or “measurement”, or alternatively “detecting” or “detection”, mean assessing the presence, absence, quantity, or amount (which can be an effective amount) of a given substance, i.e., sTREM-1, within a biological sample from a subject. “Measuring” or “measurement”, or alternatively “detecting” or “detection” as used herein include the derivation of the qualitative or quantitative concentration of said substance, i.e., sTREM-1, within the biological sample and within the subject (e.g., blood concentration or plasma concentration).

“Respiratory support” refers to any measure administered to a subject in order to compensate for a respiratory distress or failure experienced by the subject. Examples of such measures include oxygen therapy (also called standard oxygen therapy or supplemental oxygen), such as supplemental oxygen by mask, nasal cannula or nasal prongs, positive pressure, high flow nasal oxygen, non-invasive ventilation (NIV) (e.g., occlusive mask); invasive mechanical ventilation (IMV) requiring tracheal intubation and/or tracheostomy; and extracorporeal membrane oxygenation (ECMO). As used herein, “respiratory support” thus encompasses both oxygen therapy and invasive mechanical ventilation (IMV).

“Mechanical ventilation” refers to invasive respiratory support including respiratory support requiring tracheal intubation and/or tracheostomy, and extracorporeal membrane oxygenation (ECMO). As used herein, the terms “mechanical ventilation” and “invasive mechanical ventilation” are interchangeable.

“Standard of care” refers to the care routinely provided to a hospitalized subject suffering from of a disease caused by a coronavirus, in particular COVID-19 caused by SARS-CoV-2. Standard of care may include for example at least one of the following: respiratory support as defined hereinabove, vasopressor therapy (such as for example phenylephrine, norepinephrine, epinephrine, vasopressin, and/or dopamine), fluid therapy, antimicrobial therapy, antiviral therapy, cardiovascular support, renal replacement therapy, and sedation.

“Subject” refers to a mammal, preferably a human. According to the present invention, a subject is a mammal, preferably a human, suffering from a disease caused by a coronavirus, in particular from COVID-19 caused by SARS-CoV-2.

“Confirmed laboratory diagnosis of COVID-19” as used herein refers to COVID-19 caused by SARS-CoV-2 confirmed by a laboratory test such as a rRT-PCR (real-time reverse transcription polymerase chain reaction) test allowing to detect the presence of SARS-CoV-2 in a sample from a subject (such as a sample from a nasal swab, a sample from an oropharyngeal swab, a sputum sample, a lower respiratory tract aspirate, a bronchoalveolar lavage, a nasopharyngeal wash/aspirate or a nasal aspirate) or an antibody test (such as an enzyme-linked immunosorbent assay (ELISA)) allowing to detect the presence of antibodies against SARS-CoV-2 in a sample from a subject (such as a blood sample).

“Disease caused by a coronavirus” and “disease caused by a coronavirus infection” are interchangeable and refer to any symptom or set of symptoms induced in a subject by the presence of a coronavirus in the organism of said subject.

“Treating” or “Treatment” refers to a therapeutic treatment, to a prophylactic (or preventative) treatment, or to both a therapeutic treatment and a prophylactic (or preventative) treatment, wherein the object is to prevent, reduce, alleviate, and/or slow down (lessen) one or more of the symptoms or manifestations of a disease caused by a coronavirus, in particular COVID-19 caused by SARS-CoV-2, in a subject in need thereof. Symptoms of a disease caused by a coronavirus, in particular COVID-19 caused by SARS-CoV-2, include, without being limited to, a fever and respiratory symptoms such as dry cough and/or breathing difficulties that may require respiratory support (for example supplemental oxygen, non-invasive ventilation, invasive mechanical ventilation, extracorporeal membrane oxygenation (ECMO)). Manifestations of a disease caused by a coronavirus, in particular COVID-19 caused by SARS-CoV-2, also include, without being limited to, the viral load (also known as viral burden or viral titer) detected in a biological sample from the subject. In one embodiment, “treating” or “treatment” refers to a therapeutic treatment. In another embodiment, “treating” or “treatment” refers to a prophylactic or preventive treatment. In yet another embodiment, “treating” or “treatment” refers to both a prophylactic (or preventive) treatment and a therapeutic treatment.

### DETAILED DESCRIPTION

The present invention relates to triggering receptor expressed on myeloid cells-1 (TREM-1), in particular soluble triggering receptor expressed on myeloid cells-1 (sTREM-1), as a marker of a disease caused by a coronavirus (also referred to as a disease caused by a coronavirus infection), in particular as a marker of severity and/or complication(s) of said disease. The present invention also relates to a TREM-1 level, in particular a sTREM-1 level, as a marker, in particular as a marker of severity and/or complication(s) of said disease, for use in methods for identifying, assessing, and monitoring a subject suffering from a disease caused by a coronavirus as described herein.

The present invention thus relates to methods for identifying, assessing, and monitoring a subject suffering from a disease caused by a coronavirus as described herein, said methods comprising or consisting of:
measuring the level of TREM-1, in particular of sTREM-1, in a biological sample from the subject as described herein; and
comparing the level of TREM-1, in particular of sTREM-1, measured in the biological sample from the subject to a reference value as described herein.

TREM-1 (triggering receptor expressed on myeloid cells-1) is a glycoprotein receptor belonging to the Ig superfamily that is expressed notably on myeloid cells. sTREM-1 is a soluble form of TREM-1 lacking the transmembrane and intracellular domains of TREM-1. Without wishing to be bound to a theory, the Applicants suggest that PRRs (Pathogen Recognition Receptors) engagement, including Nod-like receptors (NLRs) and Toll-like receptors (TLRs), induce the upregulation of TREM-1 expression and/or its mobilization and clustering at the cell membrane, which lead to its dimerization and multimerization. Said NLRs and TLRs activation can occur by linking DAMPs (Danger Associated Molecular Patterns) or PAMPs (Pathogen Associated Molecular Patterns). In particular, said NLRs and TLRs activation can occur under sterile inflammatory conditions by linking DAMPs and/or alarmins, or under infectious conditions by linking PAMPs. This activation of NLRs and TLRs induces the upregulation of proteases, in particular of metalloproteinases, which in turn, among a number of targets, will induce the liberation of a soluble TREM-1 through proteolytic cleavage of membrane-anchored TREM-1 (Gomez-Pina et al., J Immunol. 2007 Sep. 15; 179(6):4065-73). Said proteolytic cleavage depends on the dimerization of the TREM-1 receptor. sTREM-1 is thus shed from the membrane of myeloid cells, in particular from activated myeloid cells, and sTREM-1 release is a marker of TREM-1 activation. In one embodiment, sTREM-1 corresponds to the soluble form of the extracellular domain of TREM-1. In one embodiment, sTREM-1 corresponds to a truncated TREM-1 shed from the membrane of myeloid cells, in particular from activated myeloid cells.

According to one embodiment, the level of TREM-1 is the level of TREM-1 transcript.

Example of known TREM-1 transcripts include, without being limited to, the transcript commonly referred to as TREM1-201 (transcript ID ensembl ENST00000244709.8) encoding an amino acid sequence corresponding to SEQ ID NO: 1; the transcript commonly referred to as TREM1-202, also known as TREM-1 isoform 2 (ensembl transcript ID ENST00000334475.10) encoding an amino acid sequence corresponding to SEQ ID NO: 2; the transcript commonly referred to as TREM1-207, also known as TREM-1 isoform 3 (ensembl transcript ID ENST00000591620.1) encoding an amino acid sequence corresponding to SEQ ID NO: 3, the transcript commonly referred to as TREM1-204 (ensembl transcript ID ENST00000589614.5) encoding an amino acid sequence corresponding to SEQ ID NO: 4.

In one embodiment, the TREM-1 transcript encodes an amino acid sequence as set forth in SEQ ID NO: 1, SEQ ID NO: 2, SEQ ID NO: 3 or SEQ ID NO: 4. In one embodiment, the TREM-1 transcript encodes an amino acid sequence as set forth in SEQ ID NO: 1, SEQ ID NO: 3 or SEQ ID NO: 4. In one embodiment, sTREM-1 is a variant of SEQ ID NO: 1, a variant of SEQ ID NO: 3 or a variant of SEQ ID NO: 4.

In one embodiment, a variant of SEQ ID NO: 1, SEQ ID NO: 3 or SEQ ID NO: 4 is an amino acid sequence comprising or consisting of at least 25 contiguous amino acids, preferably at least 50, 60, 70, 80, 90, 100, 110, 120, 130, 140, 150, 160, 165, 170, 175, 180 or 185 contiguous amino acids of the amino acid sequence of SEQ ID NO: 1, SEQ ID NO: 3 or SEQ ID NO: 4, respectively. In one embodiment, a variant of SEQ ID NO: 1, SEQ ID NO: 3 or SEQ ID NO: 4 is an amino acid sequence comprising or consisting of the amino acid sequence of SEQ ID NO: 1, SEQ ID NO: 3 or SEQ ID NO: 4, respectively, and additional amino acids at the C-terminus and/or at the N-terminus of SEQ ID NO: 1, SEQ ID NO: 3 or SEQ ID NO: 4, respectively, wherein the number of additional amino acids ranges from 1 to 50, preferably from 1 to 20, more preferably from 1 to 10 amino acids, such as, for example, 1, 2, 3, 4, 5, 6, 7, 8, 9 or 10 amino acids at the C-terminus and/or 1, 2, 3, 4, 5, 6, 7, 8, 9 or 10 amino acids at the N-terminus. In one embodiment, a variant of SEQ ID NO: 1, SEQ ID NO: 3 or SEQ ID NO: 4 is an amino acid sequence that typically differs from the amino acid sequence of SEQ ID NO: 1, SEQ ID NO: 3 or SEQ ID NO: 4, respectively, through one or more amino acid substitution(s), deletion(s), addition(s) and/or insertion(s). In one embodiment, said substitution(s), deletion(s), addition(s) and/or insertion(s) may affect 1, 2, 3, 4, 5, 6, 7, 8, 9, or 10 amino acids. In one embodiment, a variant of SEQ ID NO: 1, SEQ ID NO: 3 or SEQ ID NO: 4 is an amino acid sequence of at least 25 amino acids, preferably of at least 50, 60, 70, 80, 90, 100, 110, 120, 130, 140, 150, 160, 165, 170, 175, 180 or 185 amino acids, having at least 60%, 65%, 70%, 75%, 80%, 90%, 95%, or at least 96%, 97%, 98%, 99% or more identity with the amino acid sequence of SEQ ID NO: 1, SEQ ID NO: 3 or SEQ ID NO: 4, respectively.

In one embodiment, a variant of SEQ ID NO: 1, SEQ ID NO: 3 or SEQ ID NO: 4 is an amino acid sequence having at least 60%, 65%, 70%, 75%, 80%, 90%, 95%, or at least 96%, 97%, 98%, 99% or more identity with the amino acid sequence of SEQ ID NO: 1, SEQ ID NO: 3 or SEQ ID NO: 4, respectively.

According to one embodiment, the level of TREM-1 is the level of sTREM-1.

In one embodiment, sTREM-1 corresponds to the extracellular fragment generated by cleavage of the membrane-bound TREM-1 having an amino acid sequence as set forth in SEQ ID NO: 1 by a protease, preferably a matrix metallopeptidase, more preferably by the matrix metalloproteinase 9 (MMP9).

In one embodiment, sTREM-1 has an amino acid sequence as set forth in SEQ ID NO: 5 (ATKLTEEKYELKEGQTLDVKCDYTLEKFASSQKAWQIIRDG EMPKTLACTERPSKNSHPVQVGRIILEDYHDHGLLRVRMVNLQVEDSGLYQCV IYQPPKEPHMLFDRIRLVVTKGFSGTPGSNENSTQNVYKIPPTTTKALCPLYTSP RTVTQAPPKSTADVSTPDSEINLTNVTDIIRVPVFN), corresponding to amino acids 21 to 205 of SEQ ID NO: 1.

In one embodiment, sTREM-1 has an amino acid sequence as set forth in SEQ ID NO: 6 (LKEGQTLDVKCDYTLEKFASSQKAWQIIRDGEMPKTLACTE RPSKNSHPVQVGRIILEDYHDHGLLRVRMVNLQVEDSGLYQCVIYQPPKEPHM LFDRIRLVVTKGFS GTPGSNENSTQNVYKIPPTTTKALCPLYTSPRTVTQAPPKS TADVSTPDSEINLTNVTDIIRVPVFN), corresponding to amino acids 31 to 205 of SEQ ID NO: 1.

sTREM-1 may also result from an alternative splicing of TREM-1 mRNA. A TREM-1 splice variant was characterized in 2015 by Baruah et al., (J Immunol. 2015 Dec. 15; 195(12):5725-31) and was found to be secreted from primary and secondary human neutrophil granules. In one embodiment, sTREM-1 corresponds to a TREM-1 splice variant. In one embodiment, sTREM-1 corresponds to the TREM-1 transcript commonly referred to as TREM1-202, also known as TREM-1 isoform 2, encoding an amino acid sequence as set forth in SEQ ID NO: 2. In one embodiment, sTREM-1 thus has an amino acid sequence as set forth in SEQ ID NO: 2 (MRKTRLWGLLWMLFVSELRAATKLTEEKYELKEGQTLDVKCDYTLEKFAS SQ KAWQIIRDGEMPKTLACTERPSKNSHPVQVGRIILEDYHDHGLLRVRMVNLQV EDSGLYQCVIYQPPKEPHMLFDRIRLVVTKGPRCSTLSFSWLVDS)

In one embodiment, sTREM-1 comprises an amino acid sequence as set forth in SEQ ID NO: 7 (LKEGQTLDVKCDYTLEKFASSQKAWQIIRDGEMPKTLACTERPS KNSHPVQVGRIILEDYHDHGLLRVRMVNLQVEDSGLYQCVIYQPPKEPHMLFD RIRLVVTKGF), corresponding to amino acids 31 to 137 of SEQ ID NO: 1, and has a length of 200 amino acids or less, preferably of 185 amino acids or less.

In one embodiment, sTREM-1 has an amino acid sequence as set forth in SEQ ID NO: 2, SEQ ID NO: 5 or SEQ ID NO: 6. In one embodiment, sTREM-1 has an amino acid sequence as set forth in SEQ ID NO: 5 or in SEQ ID NO: 6.

In one embodiment, sTREM-1 is a variant of SEQ ID NO: 2, a variant of SEQ ID NO: 5 or a variant of SEQ ID NO: 6. In one embodiment, sTREM-1 is a variant of SEQ ID NO: 5 or a variant of SEQ ID NO: 6.

In one embodiment, a variant of SEQ ID NO: 2, SEQ ID NO: 5 or SEQ ID NO: 6 is an amino acid sequence comprising or consisting of at least 25 contiguous amino acids, preferably at least 50, 60, 70, 80, 90, 100, 110, 120, 130, 140, 150, 160, 165, 170, 175, 180 or 185 contiguous amino acids of the amino acid sequence of SEQ ID NO: 2, SEQ ID NO: 5 or SEQ ID NO: 6, respectively. In one embodiment, a variant of SEQ ID NO: 2, SEQ ID NO: 5 or SEQ ID NO: 6 is an amino acid sequence comprising or consisting of the amino acid sequence of SEQ ID NO: 2, SEQ ID NO: 5 or SEQ ID NO: 6, respectively, and additional amino acids at the C-terminus and/or at the N-terminus of SEQ ID NO: 2, SEQ ID NO: 5 or SEQ ID NO: 6, respectively, wherein the number of additional amino acids ranges from 1 to 50, preferably from 1 to 20, more preferably from 1 to 10 amino acids, such as, for example, 1, 2, 3, 4, 5, 6, 7, 8, 9 or 10 amino acids at the C-terminus and/or 1, 2, 3, 4, 5, 6, 7, 8, 9 or 10 amino acids at the N-terminus. In one embodiment, a variant of SEQ ID NO: 2, SEQ ID NO: 5 or SEQ ID NO: 6 is an amino acid sequence that typically differs from the amino acid sequence of SEQ ID NO: 2, SEQ ID NO: 5 or SEQ ID NO: 6, respectively, through one or more amino acid substitution(s), deletion(s), addition(s) and/or insertion(s). In one embodiment, said substitution(s), deletion(s), addition(s) and/or insertion(s) may affect 1, 2, 3, 4, 5, 6, 7, 8, 9, or 10 amino acids. In one embodiment, a variant of SEQ ID NO: 2, SEQ ID NO: 5 or SEQ ID NO: 6 is an amino acid sequence of at least 25 amino acids, preferably of at least 50, 60, 70, 80, 90, 100, 110, 120, 130, 140, 150, 160, 165, 170, 175, 180 or 185 amino acids, having at least 60%, 65%, 70%, 75%, 80%, 90%, 95%, or at least 96%, 97%, 98%, 99% or more identity with the amino acid sequence of SEQ ID NO: 2, SEQ ID NO: 5 or SEQ ID NO: 6, respectively. In one embodiment, a variant of SEQ ID NO: 2, SEQ ID NO: 5 or SEQ ID NO: 6 is an amino acid sequence having at least 60%, 65%, 70%, 75%, 80%, 90%, 95%, or at least 96%, 97%, 98%, 99% or more identity with the amino acid sequence of SEQ ID NO: 2, SEQ ID NO: 5 or SEQ ID NO: 6, respectively. In one embodiment, the variant of SEQ ID NO: 2, SEQ ID NO: 5 or SEQ ID NO: 6 is not SEQ ID NO: 1.

In one embodiment, sTREM-1 is a fragment of SEQ ID NO: 2, a fragment of SEQ ID NO: 5, or a fragment of SEQ ID NO: 6. In one embodiment, sTREM-1 is a fragment of SEQ ID NO: 5 or a fragment of SEQ ID NO: 6.

In one embodiment, a fragment of SEQ ID NO: 2, SEQ ID NO: 5 or SEQ ID NO: 6 is an amino acid sequence comprising or consisting of at least 25 contiguous amino acids, preferably of at least 50, 60, 70, 80, 90, 100, 110, 120, 130, 140, 150, 160, 165, 170, 175, 180 or 185 contiguous amino acids of the amino acid sequence of SEQ ID NO: 2, SEQ ID NO: 5 or SEQ ID NO: 6, respectively. In one embodiment, a fragment of SEQ ID NO: 2 is an amino acid sequence comprising or consisting of at least 25 contiguous amino acids, preferably of at least 50, 60, 70, 80, 90, 100, 110, 120, 130, 135, 140, or 145 contiguous amino acids of the amino acid sequence of SEQ ID NO: 2. In one embodiment, a fragment of SEQ ID NO: 5 is an amino acid sequence comprising or consisting of at least 25 contiguous amino acids, preferably of at least 50, 60, 70, 80, 90, 100, 110, 120, 130, 140, 150, 160, 165, 170, 175, or 180 contiguous amino acids of the amino acid sequence of SEQ ID NO: 5. In one embodiment, a fragment of SEQ ID NO: 6 is an amino acid sequence comprising or consisting of at least 25 contiguous amino acids, preferably of at least 50, 60, 70, 80, 90, 100, 110, 120, 130, 140, 150, 155, 160, 165, or 170 contiguous amino acids of the amino acid sequence of SEQ ID NO: 6.

The inventors have surprisingly shown that the level of sTREM-1 measured in a biological sample from a subject can be used as a marker of a disease caused by a coronavirus affecting said subject, in particular as a marker of severity and/or complications of said disease, as detailed in the methods described herein.

In one embodiment, the coronavirus is a human coronavirus. In one embodiment, the coronavirus is an alpha coronavirus or a beta coronavirus, preferably a beta coronavirus.

Examples of alpha coronaviruses include, without being limited to, human coronavirus 229E (HCoV-229E) and human coronavirus NL63 (HCoV-NL63) also sometimes known as HCoV-NH or New Haven human coronavirus. Examples of beta coronaviruses include, without being limited to, human coronavirus OC43 (HCoV-OC43), human coronavirus HKU1 (HCoV-HKU1), Middle East respiratory syndrome-related coronavirus (MERS-CoV) previously known as novel coronavirus 2012 or HCoV-EMC, severe acute respiratory syndrome coronavirus (SARS-CoV) also known as SARS-CoV-1 or SARS-classic, and severe acute respiratory syndrome coronavirus (SARS-CoV-2) also known as 2019-nCoV or novel coronavirus 2019.

In one embodiment, the coronavirus is selected from the group comprising or consisting of HCoV-229E, HCoV-NL63, HCoV-OC43, HCoV-HKU1, MERS-CoV, SARS-CoV-1 and SARS-CoV-2. In one embodiment, the coronavirus is selected from the group comprising or consisting of MERS-CoV, SARS-CoV-1 and SARS-CoV-2.

In one embodiment, the coronavirus is a MERS coronavirus, in particular MERS-CoV causing Middle East respiratory syndrome (MERS). Thus, in one embodiment, the subject is suffering from MERS caused by MERS-CoV.

In one embodiment, the coronavirus is a SARS coronavirus. In one embodiment, the coronavirus is SARS-CoV (also referred to as SARS-CoV-1) causing severe acute respiratory syndrome (SARS) or SARS-CoV-2 causing COVID-19. Thus, in one embodiment, the subject is suffering from SARS caused by SARS-CoV (also referred to as SARS-CoV-1) or from COVID-19 caused by SARS-CoV-2. In one embodiment, the coronavirus is SARS-CoV-2 causing COVID-19. Thus, in one in one embodiment, the subject is suffering from COVID-19 caused by SARS-CoV 2.

As used herein, “SARS-CoV-2” encompasses SARS-CoV-2 as initially identified in Wuhan, China and any variants thereof. Variants of SARS-CoV-2 may differ from each other by the presence of one or more mutation(s) in any of their proteins, including their nonstructural replicase polyproteins and their four structural proteins, known as the S (spike) protein or glycoprotein, the E (envelope) protein, the M (membrane) protein, and the N (nucleocapsid) protein. In particular, variants of SARS-CoV-2 may differ from each other by the presence of one or more mutation(s) in their S protein. The reference sequence of the S protein, consisting of 1273 amino acids, is as set forth in SEQ ID NO: 14, corresponding to UniProtKB accession number P0DTC2, last modified on Apr. 22, 2020.

As indicated by the US Centers for Disease Control and Prevention (CDC), examples of SARS-CoV-2 variants include, without being limited to:
variant B.1.1.7, also known as Alpha (WHO label), VUI—202012/01, VOC-202012/01, 20I/501Y.V1, or colloquially as the “UK variant or British variant or English variant”, comprising the following mutations (based on the sequence SEQ ID NO: 21): 69del, 70del, 144del, N501Y, A570D, D614G, P681H, T716I, S982A, D1118H, and optionally E484K, S494P, and/or K1191N;
variant B.1.351, also known as Beta (WHO label), 20H/501Y.V2 (formerly 20C/501Y.V2), or colloquially as the “South African” variant, comprising the following mutations (based on the sequence SEQ ID NO: 21): D80A, D215G, 241del, 242del, 243del, K417N, E484K, N501Y, D614G, A701V;
variant P.1, also known as Gamma (WHO label), 20J/501Y.V3, or colloquially as the “Brazilian variant”, comprising the following mutations (based on the sequence SEQ ID NO: 21): L18F, T20N, P26S, D138Y, R1905, K417T, E484K, N501Y, D614G, H655Y, T1027I;
variant P.2, also known as Zeta (WHO label) or 20J, a variant first detected in Brazil, comprising the following mutations (based on the sequence SEQ ID NO: 21): E484K, D614G, V1176F, and optionally F565L;
variant B.1.617, also known as 20A/484Q, or colloquially as the “Indian variant”, comprising the following mutations (based on the sequence SEQ ID NO: 21): L452R, E484Q, D614G;
variant B.1.617.1, also known as Kappa (WHO label) or 20A/S:154K, a variant first detected in India, comprising the following mutations (based on the sequence SEQ ID NO: 21): G142D, E154K, L452R, E484Q, D614G, P681R, Q1071H, and optionally T95I;
variant B.1.617.2, also known as Delta (WHO label) or 20A/S:478K, a variant first detected in India, comprising the following mutations (based on the sequence SEQ ID NO: 21): T19R, 156del, 157del, R158G, L452R, T478K, D614G, P681R, D950N, and optionally G142D;
variant B.1.617.3, a variant first detected in India, comprising the following mutations (based on the sequence SEQ ID NO: 21): T19R, G142D, L452R, E484Q, D614G, P681R, D950N;
variant B.1.427, also known as Epsilon (WHO label) or 20C/S:452R, comprising the following mutations (based on the sequence SEQ ID NO: 21): L452R, D614G;
variant B.1.429, also known as 20C/S:452R, comprising the following mutations (based on the sequence SEQ ID NO: 21): S13I, W152C, L452R, D614G;
variant B.1.525, also known as Eta (WHO label) or 20A/S:484K, comprising the following mutations (based on the sequence SEQ ID NO: 21): A67V, 69del, 70del, 144del, E484K, D614G, Q677H, F888L;
variant B.1.526 also known as Iota (WHO label) or 20C/S:484K, comprising the following mutations (based on the sequence SEQ ID NO: 21): T95I, D253G, D614G, and optionally LSF, S477N, E484K, and/or A701V; and
variant B.1.526.1, also known as 20C, comprising the following mutations (based on the sequence SEQ ID NO: 21): D80G, 144del, F157S, L452R, D614G, D950H, and optionally T791I and/or T859N.

Thus, in one embodiment, the subject is suffering from COVID-19 caused by SARS-CoV-2 or any variant of SARS-CoV-2. In one embodiment, the subject is suffering from COVID-19 caused by a SARS-CoV-2 variant selected from the group comprising or consisting of variant B.1.1.7 (Alpha), variant B.1.351 (Beta), variant P.1 (Gamma), variant P.2 (Zeta), variant B.1.617, variant B.1.617.1 (Kappa), variant B.1.617.2 (Delta) and/or variant B.1.617.3. In one embodiment, the subject is suffering from COVID-19 caused by the SARS-CoV-2 variant B.1.1.7 (Alpha). In one embodiment, the subject is suffering from COVID-19 caused by the SARS-CoV-2 variant B.1.617, or any of the related variants B.1.617.1 (Kappa), B.1.617.2 (Delta) and/or B.1.617.3.

In one embodiment, the subject suffering from a disease caused by a coronavirus as described hereinabove is not hospitalized. In one embodiment, the subject suffering from a disease caused by a coronavirus as described hereinabove is hospitalized.

In one embodiment, the subject suffering from a disease caused by a coronavirus as described hereinabove is hospitalized but does not require admission in intensive care unit (ICU). In one embodiment, the subject suffering from a disease caused by a coronavirus as described hereinabove is hospitalized and requires admission in ICU. In one embodiment, the subject suffering from a disease caused by a coronavirus as described hereinabove is hospitalized in ICU.

In one embodiment, the subject suffering from a disease caused by a coronavirus as described hereinabove is hospitalized and does not require respiratory support. In one embodiment, the subject suffering from a disease caused by a coronavirus as described hereinabove is hospitalized and requires respiratory support.

In one embodiment, the subject suffering from a disease caused by a coronavirus as described hereinabove is hospitalized and requires non-invasive ventilation (NIV). In one embodiment, the subject suffering from a disease caused by a coronavirus as described hereinabove is hospitalized and requires invasive mechanical ventilation (IMV). In one embodiment, the subject suffering from a disease caused by a coronavirus as described hereinabove is hospitalized in ICU and requires respiratory support. In one embodiment, the subject suffering from a disease caused by a coronavirus as described hereinabove is hospitalized in ICU and requires IMV. In one embodiment, the subject suffering from a disease caused by a coronavirus as described hereinabove is hospitalized in ICU and is under IMV. In one embodiment, the subject suffering from a disease caused by a coronavirus as described hereinabove is hospitalized in ICU and has been under IMV for less than 6 hours, 12 hours, 18 hours, 24 hours, 30 hours, 36 hours, 42 hours, or 48 hours, preferably for less than 48 hours.

In one embodiment, the subject suffers from acute respiratory failure or from acute respiratory distress syndrome (ARDS) associated to the disease caused by a coronavirus as described hereinabove.

In one embodiment, the subject is a male. In one embodiment, the subject is a female.

In one embodiment the subject is an adult. Thus, in one embodiment, the subject is older than 18, 19, 20 or 21 years of age. In one embodiment the subject is a child. Thus, in one embodiment, the subject is younger 18, 17, 16 or 15 years of age.

In one embodiment, the subject is younger than 85, 80, 75, 70, 65 or 60 years of age. In one embodiment, the subject is 85 years old or younger. In one embodiment, the subject is older than 60, 65 or 70 years of age. In one embodiment, the subject is older than 60, 65 or 70 years of age and younger than 85 years of age. In one embodiment, the subject is 60 years old or older. In one embodiment, the subject is 60 years old or older and younger than 85 years old.

In one embodiment, the subject suffers from at least one comorbidity.

As used herein, “comorbidity” refers to a disease or condition coexisting in the subject with the disease caused by a coronavirus.

Examples of comorbidities that may coexist in the subject with a disease caused by a coronavirus include, without being limited to, asthma, autoimmune or auto-inflammatory diseases or conditions, cardiovascular diseases or conditions, chronic bronchitis, chronic kidney diseases, chronic liver disease, chronic obstructive pulmonary disease (COPD), cystic fibrosis, diabetes, emphysema, high blood pressure, immunodeficiency, malignancy (i.e., cancer), obesity, pulmonary hypertension, and severe respiratory conditions.

In one embodiment, the subject presents at least one comorbidity selected from the group comprising or consisting of asthma, autoimmune or auto-inflammatory diseases or conditions, cardiovascular diseases or conditions, chronic bronchitis, chronic kidney diseases, chronic liver disease, chronic obstructive pulmonary disease (COPD), cystic fibrosis, diabetes, emphysema, high blood pressure, immunodeficiency, malignancy (i.e., cancer), obesity, pulmonary hypertension, and severe respiratory conditions.

According to the methods as described herein, the level of TREM-1, in particular the level of sTREM-1, is measured in a biological sample from a subject as described hereinabove.

As used herein, “biological sample” refers to a biological sample isolated, collected or harvested from a subject and can include, by way of example and not limitation, bodily fluids, cell samples and/or tissue extracts such as homogenates or solubilized tissues obtained from a subject.

In one embodiment, the present invention does not comprise obtaining a biological sample from a subject. Thus, in one embodiment, the biological sample from the subject is a biological sample previously obtained from the subject. Said biological sample may be conserved in adequate conditions before being used as described herein.

In one embodiment, the biological sample from the subject is a body fluid sample. Examples of body fluids include, without being limited to, blood, plasma, serum, lymph, urine, bronchioalveolar lavage fluid, cerebrospinal fluid, sweat or any other bodily secretion or derivative thereof.

According to the present invention, “blood” includes whole blood, plasma, serum, circulating epithelial cells, constituents, or any other derivative of blood.

In one embodiment, the biological sample from the subject is a blood sample. In one embodiment, the biological sample from the subject is a whole blood sample or a plasma sample. Methods for obtaining a plasma sample are routinely used in clinical laboratories. In one embodiment, the whole blood sample or the plasma sample from the subject is processed to obtain a serum sample. Methods for obtaining a serum sample from a whole blood sample or a plasma sample are routinely used in clinical laboratories.

In one embodiment, the biological sample is a blood sample, a plasma sample or a serum sample. Accordingly, in one embodiment, the TREM-1 level, in particular the sTREM-1 level is a blood level, a plasma level, or a serum level.

In one embodiment, the biological sample from the subject is a tissue extract. Tissue extracts are obtained routinely from tissue biopsy and autopsy material.

As used herein, the term “level” as in “TREM-1 level”, and in particular “sTREM-1 level”, refers to the expression level of TREM-1, in particular of sTREM-1. It can refer alternatively to the transcription level of TREM-1, in particular of sTREM-1 (i.e., the level of mRNA or cDNA) or to the translation level of TREM-1, in particular of sTREM-1 (i.e., the level of protein). The expression level may be detected intracellularly or extracellularly.

According to the present invention, the level of TREM-1, in particular of sTREM-1, may be measured by any known method in the art. Methods for measuring an expression level such as a transcription level or a translation level are well-known to the skilled artisan.

In one embodiment, the term “level” as in “TREM-1 level”, and in particular “sTREM-1 level”, refers to the quantity, amount, or concentration of TREM-1, in particular of sTREM-1. Thus, the level of TREM-1, in particular of sTREM-1, measured in a biological sample from a subject refers to the quantity, amount, or concentration of TREM-1, in particular of sTREM-1, in said biological sample.

According to one embodiment, the level of TREM-1, in particular of sTREM-1, refers to a protein level, a protein quantity, a protein amount, or a protein concentration.

In one embodiment, the level of TREM-1 refers to the level of an amino acid sequence as set forth in SEQ ID NO: 1, SEQ ID NO: 3 and/or SEQ ID NO: 4, and/or variants thereof as described hereinabove.

In one embodiment, the level of TREM-1 is a level of sTREM-1.

In one embodiment, the level of sTREM-1 refers to the level of an amino acid sequence as set forth in SEQ ID NO: 2, SEQ ID NO: 5 and/or SEQ ID NO: 6, and/or fragments and/or variants thereof as described hereinabove. In one embodiment, the level of sTREM-1 refers to the level of an amino acid sequence as set forth in SEQ ID NO: 5 and/or SEQ ID NO: 6, and/or fragments and/or variants thereof as described hereinabove.

Methods for measuring the translation level of TREM-1, in particular of sTREM-1, (i.e., the level of TREM-1 protein or of sTREM-1 protein) are well-known to the skilled artisan and include, without being limited to, immunohistochemistry, multiplex methods (such as Luminex®), immunoassays, western blot, enzyme-linked immunosorbent assay (ELISA), sandwich ELISA, multiplex ELISA, capillary-based ELISA (such as the ELLA® platform), electrochemiluminescence (ECL) also referred as electrogenerated chemiluminescence or electrochemiluminescence immunoassay (ECLIA), enzyme-linked fluorescent assay (ELFA), fluorescent-linked immunosorbent assay (FLISA), enzyme immunoassay (EIA), radioimmunoassay (RIA), flow cytometry (FACS), surface plasmon resonance (SPR), biolayer interferometry (BLI), immunochromatographic assay (ICA) (such as NEXUS IB10, Sphingotech) and mass spectrometry-based approaches.

Typically, measuring the level of TREM-1 protein, in particular of sTREM-1 protein, in a biological sample as described hereinabove may comprise contacting the biological sample with a binding partner capable of selectively interacting with TREM-1, or sTREM-1, in the biological sample.

In one embodiment, measuring the level of TREM-1 protein, in particular of sTREM-1 protein, in a biological sample as described hereinabove comprises the use of an antibody, such as a polyclonal or a monoclonal antibody.

Examples of antibodies allowing the detection of TREM-1, in particular of sTREM-1, include, without being limited to, the polyclonal antibody raised against Metl-Arg200 amino acids of human TREM-1 (reference AF1278 from R&D Systems), the monoclonal antibody raised against Ala21-Asn205 of human TREM-1 (reference MAB1278 from R&D Systems), the purified anti-human CD354 (TREM-1) antibody (clone TREM-26, reference 314902 from BioLegend), the purified anti-human CD354 (TREM-1) antibody (clone TREM-37, reference 316102 from BioLegend), the monoclonal mouse anti-human sTREM1 (clone 15G7, reference 298099 from USBio), the mouse anti-human TREM1 (clone 2E2, reference 134704 from USBio). Other non-limitative examples of antibodies allowing the detection of sTREM-1 include sTREM-1 and/or TREM-1 antibodies described in the following patents or patent applications: US2013/150559, US 2013/211050, US 2013/309239, WO2013/120553 and U.S. Pat. No. 8,106,165.

In one embodiment, measuring the level of TREM-1, in particular of sTREM-1, in particular the level of sTREM-1 protein, in a biological sample as described hereinabove comprises the use of an ELISA, an ECLIA or an ELFA.

An ELISA may thus be used for measuring the level of TREM-1, in particular of sTREM-1, in a biological sample, wherein for example the wells of an assay plate are coated with at least one antibody which recognizes TREM-1, or sTREM-1. A biological sample containing or suspected of containing TREM-1, or sTREM-1, is then added to the coated wells. After a period of incubation sufficient to allow the formation of antibody-TREM-1 complexes, or antibody-sTREM-1 complexes, the plate can be washed to remove unbound moieties and a detectably labelled secondary binding molecule added, such as, for example, a second antibody which recognizes TREM-1, or sTREM-1, coupled to horseradish peroxidase (HRP). The secondary binding molecule is allowed to react with any captured antibody-TREM-1 complexes, or antibody-sTREM-1 complexes, the plate washed and the presence of the secondary binding molecule detected using methods well-known in the art. It is understood that commercial assay enzyme-linked immunosorbent assay (ELISA) kits are available. Examples of ELISAs thus include, without being limited to, the TREM-1 Quantikine ELISA kit (reference DTRM10C from R&D Systems); the human TREM-1 DuoSet (references DY1278B and DY1278BE from R&D Systems), the sTREM-1 ELISA (reference sTREM-1 ELISA from iQProducts).

An ECLIA may also be used for measuring the level of TREM-1, in particular of sTREM-1, in a biological sample, wherein for example a biological sample containing or suspected of containing TREM-1, or sTREM-1, is incubated with at least two antibodies which recognize TREM-1, or sTREM-1, on different epitopes in order to form sandwich antibodies-TREM-1 or antibodies-sTREM-1 complexes, with one of the antibody being biotinylated (i.e., the capture antibody) and the other being labeled with a ruthenium complex (i.e., the detection antibody). After a period of incubation sufficient to allow the formation of the sandwich complexes, streptavidin-coated microparticles (such as streptavidin-coated magnetic beads) are added so that the sandwich complexes become bound to the particles via interaction of biotin and streptavidin. Once in the measuring cell of an analyzer, the microparticles are magnetically captured onto the surface of an electrode. Unbound moieties are removed and a voltage is applied to the electrode, thus exciting the ruthenium complex which then emits light at 620 nm. The light emitted is measured by a photomultiplier in the measuring cell of the analyzer. Examples of such ECLIAs include Elecsys® (Roche Diagnostics).

An ELFA may also be used for measuring the level of TREM-1, in particular of sTREM-1, in a biological sample, wherein for example a receptacle is coated with at least one antibody which recognizes TREM-1, or sTREM-1. A biological sample containing or suspected of containing TREM-1, or sTREM-1, is then added to the receptacle. After a period of incubation sufficient to allow the formation of antibody-TREM-1 complexes, or antibody-sTREM-1 complexes, the receptacle can be washed to remove unbound moieties and a secondary binding molecule labeled with an enzyme (such as alkaline phosphatase) is added. The secondary binding molecule is allowed to react with any captured antibody-TREM-1 complexes, or antibody-sTREM-1 complexes, the receptacle washed and the presence of the secondary binding molecule detected through the measurement of the fluorescence emitted upon addition of a substrate of the enzyme (such as 4-methyl-umbelliferyl phosphate), which becomes fluorescent after hydrolysis by the enzyme. Examples of ELFAs include VIDAS® (Biomérieux).

According to one embodiment, the level of TREM-1, in particular sTREM-1, refers to a nucleic acid level, a nucleic acid quantity, a nucleic acid amount or a nucleic acid concentration. In one embodiment, the nucleic acid is a RNA, preferably a mRNA, or a cDNA.

In one embodiment, the level of TREM-1 is a level of TREM-1 transcript.

In one embodiment, the level of TREM-1 nucleic acid refers to the level of mRNA or cDNA encoding an amino acid sequence as set forth in SEQ ID NO: 1, SEQ ID NO: 3 and/or SEQ ID NO: 4, and/or variants thereof as described hereinabove.

In one embodiment, the level of TREM-1 is a level of sTREM-1.

In one embodiment, the level of sTREM-1 nucleic acid refers to the level of mRNA or cDNA encoding an amino acid sequence as set forth in SEQ ID NO: 2, SEQ ID NO: 5 and/or SEQ ID NO: 6, and/or fragments and/or variants thereof as described hereinabove. In one embodiment, the level of sTREM-1 nucleic acid refers to the level of mRNA or cDNA encoding an amino acid sequence as set forth in SEQ ID NO: 5 and/or SEQ ID NO: 6, and/or fragments and/or variants thereof as described hereinabove.

Methods for measuring the transcription level of TREM-1, in particular of sTREM-1, (i.e., the level of TREM-1 mRNA or cDNA, in particular of sTREM-1 mRNA or cDNA) in a biological sample as described hereinabove are well-known to the skilled artisan and include, without being limited to, PCR, qPCR, RT-PCR, RT-qPCR, northern blot, hybridization techniques such as, for example, use of microarrays, and combination thereof including but not limited to, hybridization of amplicons obtained by RT-PCR, sequencing such as, for example, next-generation DNA sequencing (NGS) or RNA-seq (also known as “Whole Transcriptome Shotgun Sequencing”).

In one embodiment, the TREM-1 nucleic acid level, in particular the sTREM-1 nucleic acid level, is measured using the forward and reverse primers having a nucleotide sequence has set forth in SEQ ID NO: 8 and SEQ ID NO: 9, respectively. In one embodiment, the TREM-1 nucleic acid level, in particular the sTREM-1 nucleic acid level, is measured using the forward and reverse primers having a nucleotide sequence has set forth in SEQ ID NO: 10 and SEQ ID NO: 11, respectively. In one embodiment, the TREM-1 nucleic acid level, in particular the sTREM-1 nucleic acid level, is measured using the forward and reverse primers having a nucleotide sequence has set forth in SEQ ID NO: 12 and SEQ ID NO: 13, respectively.

In one embodiment, the level of TREM-1, in particular of sTREM-1, measured in a biological sample as described hereinabove is the level at baseline, i.e., a baseline TREM-1 level, in particular a baseline sTREM-1 level. In one embodiment, a baseline level is the level of TREM-1, in particular of sTREM-1, measured in a biological sample obtained from the subject before the start of medical care, before or at the beginning of the administration of a therapy, upon hospitalization, upon admission in intensive care unit (ICU) or upon start of mechanical ventilation. In one embodiment, a baseline level is the level of TREM-1, in particular of sTREM-1, measured after diagnosis of a disease caused by a coronavirus as described hereinabove, in particular after diagnosis in the hospital of a disease caused by a coronavirus as described hereinabove. In one embodiment, a baseline level is the level of TREM-1, in particular of sTREM-1, measured on the first day of hospitalization. In one embodiment, a baseline level is the level of TREM-1, in particular of sTREM-1, measured on the first day of hospitalization in ICU. In one embodiment, a baseline level is the level of TREM-1, in particular of sTREM-1, measured on the first day of mechanical ventilation.

In one embodiment, the level of TREM-1, in particular of sTREM-1, measured in a biological sample as described hereinabove is the level of TREM-1, in particular of sTREM-1, measured less than 12 h, 24 h, 36 h, 48 h, 60 h or 72 h following hospitalization (or measured in a biological sample as described hereinabove obtained from the subject less than 12 h, 24 h, 36 h, 48 h, 60 h or 72 h following hospitalization), in particular following admission in intensive care unit (ICU), or following the start of mechanical ventilation. In one embodiment, the level of TREM-1, in particular of sTREM-1, measured in a biological sample as described hereinabove is the level of TREM-1, in particular of sTREM-1, measured 12 h, 24 h, 36 h, 48 h, 60 h or 72 h following hospitalization (or measured in a biological sample as described hereinabove obtained from the subject 12 h, 24 h, 36 h, 48 h, 60 h or 72 h following hospitalization), in particular following admission in intensive care unit (ICU), or following the start of mechanical ventilation.

In one embodiment, the level of TREM-1, in particular of sTREM-1, measured in a biological sample as described hereinabove is the level of TREM-1, in particular of sTREM-1, measured on day 1, 2, 3, 4, 5, 6, 7, 8, 9, 10, 11, 12, 13, 14, 15, 16, 17, 18, 19, or 21 following hospitalization (or measured in a biological sample as described hereinabove obtained from the subject on day 1, 2, 3, 4, 5, 6, 7, 8, 9, 10, 11, 12, 13, 14, 15, 16, 17, 18, 19, 20 or 21 following hospitalization), in particular following admission in intensive care unit (ICU), or following the start of mechanical ventilation. In other words, in one embodiment, the level of TREM-1, in particular of sTREM-1, measured in a biological sample as described hereinabove is the level of TREM-1, in particular of sTREM-1, measured on the first, second, third, fourth, fifth, sixth, or seventh day following hospitalization (or measured in a biological sample as described hereinabove obtained from the subject on the first, second, third, fourth, fifth, sixth, or seventh day following hospitalization), in particular following admission in intensive care unit (ICU), or following the start of mechanical ventilation.

In one embodiment, the level of TREM-1, in particular of sTREM-1, measured in a biological sample as described hereinabove is the level of TREM-1, in particular of sTREM-1, measured on day 3 following hospitalization (or measured in a biological sample as described hereinabove obtained from the subject on day 3 following hospitalization), in particular following admission in intensive care unit (ICU), or following the start of mechanical ventilation. In other words, in one embodiment, the level of TREM-1, in particular of sTREM-1, measured in a biological sample as described hereinabove is the level of TREM-1, in particular of sTREM-1, measured on the third day following hospitalization (or measured in a biological sample as described hereinabove obtained from the subject on the third day following hospitalization), in particular following admission in intensive care unit (ICU), or following the start of mechanical ventilation.

According to the methods as described herein, the level of TREM-1, in particular of sTREM-1, measured in a biological sample from a subject as described hereinabove is compared to a reference value.

According to one embodiment, the reference value is a reference TREM-1 level, in particular a reference sTREM-1 level, preferably a blood, plasma or serum level.

According to one embodiment, the reference TREM-1 level, in particular the reference sTREM-1 level, is determined using an enzyme-linked immunosorbent assay (ELISA).

According to one embodiment, the reference TREM-1 level, in particular the reference sTREM-1 level, as determined using a given method or assay encompasses corresponding reference TREM-1 levels, in particular corresponding reference sTREM-1 levels, as determined using another method or assay. Starting from levels obtained with a given method or assay, the skilled artisan will know how to determine corresponding levels obtained with another method or assay. Methods to do so include for example (i) measuring the levels with two different methods or assays in the samples obtained from the subjects of a given reference population, such as a reference population as described herein, thus obtaining two sets of measures for said given reference population; and (ii) determining the correlation between the two sets of measures obtained for the given reference population.

In one embodiment, the reference TREM-1 level, in particular the reference sTREM-1 level, as determined using an ELISA encompasses corresponding reference TREM-1 levels, in particular corresponding reference sTREM-1 levels, as determined using another immunoassay, such as an electrochemiluminescence immunoassay (ECLIA) or an enzyme-linked fluorescence assay (ELFA). In one embodiment, the reference TREM-1 level, in particular the reference sTREM-1 level, as determined using an ELISA encompasses the corresponding reference TREM-1 level, in particular the corresponding reference sTREM-1 level, as determined using an ECLIA.

In one embodiment, the reference value is derived from a reference population. In one embodiment, the reference value is derived from population studies, including, for example, subjects having a similar age range, or subjects in the same or similar ethnic group.

According to one embodiment, the reference value is derived from the measure of the TREM-1 level, in particular the sTREM-1 level, in a biological sample obtained from one or more subjects who are substantially healthy. In one embodiment, a “substantially healthy subject” is a subject who has not been diagnosed or identified as having or suffering from a disease caused by a coronavirus. In one embodiment, a “substantially healthy subject” is a subject who has not been diagnosed or identified as having or suffering from a disease caused by a coronavirus or any other infection. In one embodiment, a “substantially healthy subject” is a subject who has not been diagnosed or identified as having or suffering from a disease caused by a coronavirus or any other disease inducing a response from the immune system or any other disease inducing activation of the TREM-1 pathway. Thus, in one embodiment, the reference value is a reference TREM-1 level, in particular a reference sTREM-1 level, derived from a reference population of subjects who are substantially healthy.

In one embodiment, the reference value derived from a reference population of subjects who are substantially healthy is a TREM-1 level, in particular a sTREM-1 level, preferably a blood, plasma or serum level, ranging from about 20 pg/mL to about 500 pg/mL, preferably from about 50 pg/mL to about 250 pg/mL, more preferably from about 100 pg/mL to about 200 pg/mL. In one embodiment, the reference value derived from a reference population of subjects who are substantially healthy is a TREM-1 level, in particular a sTREM-1 level, preferably a blood, plasma or serum level, ranging from about 20 pg/mL to about 500 pg/mL, preferably from about 50 pg/mL to about 250 pg/mL, more preferably from about 100 pg/mL to about 200 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value derived from a reference population of subjects who are substantially healthy is a TREM-1 level, in particular a sTREM-1 level, preferably a blood, plasma or serum level, ranging from about 50 pg/mL to about 500 pg/mL, preferably from about 200 pg/mL to about 450 pg/mL, more preferably from about 250 pg/mL to about 400 pg/mL, as determined using an ECLIA, or a corresponding TREM-1 level, in particular a corresponding sTREM-1 level, preferably a blood, plasma or serum level, as determined using another immunoassay, in particular an ELISA.

In one embodiment, the reference value derived from a reference population of subjects who are substantially healthy is a TREM-1 level, in particular a sTREM-1 level, preferably a blood, plasma or serum level, of about 50, 75, 100, 125, 150, 175, 200, 225, or 250 pg/mL. In one embodiment, the reference value derived from a reference population of subjects who are substantially healthy is a TREM-1 level, in particular a sTREM-1 level, preferably a blood, plasma or serum level, of about 50, 75, 100, 125, 150, 175, 200, 225, or 250 pg/mL as determined using an ELISA, or a corresponding TREM-1 level, in particular a corresponding sTREM-1 level, preferably a blood, plasma or serum level, as determined using another immunoassay, in particular an ECLIA. In one embodiment, the reference value derived from a reference population of subjects who are substantially healthy is a TREM-1 level, in particular a sTREM-1 level, preferably a blood, plasma or serum level, of about 50, 55, 60, 65, 70, 75, 80, 85, 90, 95, 100, 105, 110, 115, 120, 125, 130, 135, 140, 145, or 150 pg/mL. In one embodiment, the reference value derived from a reference population of subjects who are substantially healthy is a TREM-1 level, in particular a sTREM-1 level, preferably a blood, plasma or serum level, of about 50, 55, 60, 65, 70, 75, 80, 85, 90, 95, 100, 105, 110, 115, 120, 125, 130, 135, 140, 145, or 150 pg/mL as determined using an ELISA, or a corresponding TREM-1 level, in particular a corresponding sTREM-1 level, preferably a blood, plasma or serum level, as determined using another immunoassay, in particular an ECLIA. In one embodiment, the reference value derived from a reference population of subjects who are substantially healthy is a TREM-1 level, in particular a sTREM-1 level, preferably a blood, plasma or serum level, of about 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 pg/mL. In one embodiment, the reference value derived from a reference population of subjects who are substantially healthy is a TREM-1 level, in particular a sTREM-1 level, preferably a blood, plasma or serum level, of about 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 pg/mL as determined using an ELISA, or a corresponding TREM-1 level, in particular a corresponding sTREM-1 level, preferably a blood, plasma or serum level, as determined using another immunoassay, in particular an ECLIA.

In one embodiment, the reference value derived from a reference population of subjects who are substantially healthy is a TREM-1 level, in particular a sTREM-1 level, preferably a blood, plasma or serum level, of about 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355; 360, 365, 370, 375, 380, 385, 390, 395, 400, 405, 410, or 415 pg/mL as determined using an ECLIA, or a corresponding TREM-1 level, in particular a corresponding sTREM-1 level, preferably a blood, plasma or serum level, as determined using another immunoassay, in particular an ELISA.

According to one embodiment, the reference value is derived from the measure of the TREM-1 level, in particular of the sTREM-1 level, in a biological sample from one or more subjects diagnosed or identified as suffering, or having suffered, from a disease caused by a coronavirus, in particular from COVID-19 caused by SARS-CoV-2. Thus, in one embodiment, the reference value is a reference TREM-1 level, in particular a reference sTREM-1 level, derived from a reference population of subjects diagnosed or identified as suffering, or having suffered, from a disease caused by a coronavirus, in particular from COVID-19 caused by SARS-CoV-2.

In one embodiment, the reference value can be derived from statistical analyses and/or risk prediction data of a reference population as described hereinabove obtained from mathematical algorithms and computed indices of a disease caused by a coronavirus, in particular COVID-19 caused by SARS-CoV-2.

In one embodiment, the reference value derived from a reference population as described hereinabove is the average TREM-1 level, in particular the average sTREM-1 level, of said reference population. In one embodiment, the reference value derived from a reference population as described hereinabove is the median TREM-1 level, in particular the median sTREM-1 level, of said reference population.

In one embodiment, the reference value derived from a reference population as described hereinabove is a TREM-1 tercile (or tertile), in particular a sTREM-1 tercile (or tertile), i.e., the first TREM-1 tercile, in particular the first sTREM-1 tercile, or the second TREM-1 tercile, in particular the second sTREM-1 tercile, of said reference population.

According to this embodiment of the present invention:
the first tercile (or tertile) corresponds to the TREM-1 value or the sTREM-1 value below which a third of the TREM-1 or sTREM-1 levels measured in the reference population lie and above which two thirds of the TREM-1 or sTREM-1 levels measured in the reference population lie; and
the second tercile (or tertile) corresponds to the TREM-1 value or the sTREM-1 value below which two thirds of the TREM-1 or sTREM-1 levels measured in the reference population lie and above which one third of the TREM-1 or sTREM-1 levels measured in the reference population lie.

In one embodiment, the reference value, preferably the reference value derived from a reference population as described hereinabove, is a TREM-1 level, in particular a sTREM-1 level, preferably a blood, plasma or serum level, ranging from about 20 pg/mL to about 6000 pg/mL, preferably from about 30 pg/mL to about 2000 pg/mL, more preferably from about 50 pg/mL to about 1000 pg/mL. In one embodiment, the reference value, preferably the reference value derived from a reference population as described hereinabove, is a TREM-1 level, in particular a sTREM-1 level, preferably a blood, plasma or serum level, ranging from about 20 pg/mL to about 6000 pg/mL, preferably from about 30 pg/mL to about 2000 pg/mL, more preferably from about 50 pg/mL to about 1000 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value, preferably the reference value derived from a reference population as described hereinabove, is a TREM-1 level, in particular a sTREM-1 level, preferably a blood, plasma or serum level, ranging from about 50 pg/mL to about 800 pg/mL, preferably from about 75 pg/mL to about 600 pg/mL, more preferably from about 100 pg/mL to about 400 pg/mL, even more preferably from about 130 pg/mL to about 400 pg/mL. In one embodiment, the reference value, preferably the reference value derived from a reference population as described hereinabove, is a TREM-1 level, in particular a sTREM-1 level, preferably a blood, plasma or serum level, ranging from about 50 pg/mL to about 800 pg/mL, preferably from about 75 pg/mL to about 600 pg/mL, more preferably from about 100 pg/mL to about 400 pg/mL, even more preferably from about 130 pg/mL to about 400 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value, preferably the reference value derived from a reference population as described hereinabove, is a TREM-1 level, in particular a sTREM-1 level, preferably a blood, plasma or serum level, ranging from about 130 pg/mL to about 600 pg/mL, preferably from about 200 pg/mL to about 500 pg/mL, more preferably from about 250 pg/mL to about 400 pg/mL, even more preferably from about 300 pg/mL to about 375 pg/mL. In one embodiment, the reference value, preferably the reference value derived from a reference population as described hereinabove, is a TREM-1 level, in particular a sTREM-1 level, preferably a blood, plasma or serum level, ranging from about 130 pg/mL to about 600 pg/mL, preferably from about 200 pg/mL to about 500 pg/mL, more preferably from about 250 pg/mL to about 400 pg/mL, even more preferably from about 300 pg/mL to about 375 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value, preferably the reference value derived from a reference population as described hereinabove, is a TREM-1 level, in particular a sTREM-1 level, preferably a blood, plasma or serum level, ranging from about 350 pg/mL to about 1200 pg/mL, preferably from about 600 pg/mL to about 1100 pg/mL, more preferably from about 700 pg/mL to about 1000 pg/mL, as determined using an ECLIA, or a corresponding TREM-1 level, in particular a corresponding sTREM-1 level, preferably a blood, plasma or serum level, as determined using another immunoassay, in particular an ELISA.

In one embodiment, the reference value, preferably the reference value derived from a reference population as described hereinabove, is a TREM-1 level, in particular a sTREM-1 level, preferably a blood, plasma or serum level, of about 75, 100, 125, 150, 175, 200, 225, 250, 275, 300, 325, 350, 375 or 400 pg/mL. In one embodiment, the reference value, preferably the reference value derived from a reference population as described hereinabove, is a TREM-1 level, in particular a sTREM-1 level, preferably a blood, plasma or serum level, of about 75, 100, 125, 150, 175, 200, 225, 250, 275, 300, 325, 350, 375 or 400 pg/mL as determined using an ELISA, or a corresponding TREM-1 level, in particular a corresponding sTREM-1 level, preferably a blood, plasma or serum level, as determined using another immunoassay, in particular an ECLIA. In one embodiment, the reference value, preferably the reference value derived from a reference population as described hereinabove, is a TREM-1 level, in particular a sTREM-1 level, preferably a blood, plasma or serum level, of about 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, or 430 pg/mL. In one embodiment, the reference value, preferably the reference value derived from a reference population as described hereinabove, is a TREM-1 level, in particular a sTREM-1 level, preferably a blood, plasma or serum level, of about 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 pg/mL.

In one embodiment, the reference value, preferably the reference value derived from a reference population as described hereinabove, is a TREM-1 level, in particular a sTREM-1 level, preferably a blood, plasma or serum level, of about 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 885, 890, 895, 900, 905, 910, 915, 920, 925, 930, 935, 940, 945, 950, 955, 960, 965, 970, 975, 980, 985, 990, 995, or 1000 pg/mL as determined using an ECLIA, or a corresponding TREM-1 level, in particular a corresponding sTREM-1 level, preferably a blood, plasma or serum level, as determined using another immunoassay, in particular an ELISA.

According to one embodiment, the reference value is a personalized reference value, i.e., the reference value is a TREM-1 level, in particular a sTREM-1 level, measured in a biological sample obtained from the subject.

In one embodiment, the personalized reference value is a TREM-1 level, in particular a sTREM-1 level, measured in a biological sample obtained from the subject at baseline, i.e., a baseline TREM-1 level, in particular a baseline sTREM-1 level. In one embodiment, the baseline level is a TREM-1 level, in particular a sTREM-1 level, measured in a biological sample obtained from the subject before the start of medical care. In one embodiment, the baseline level is a TREM-1 level, in particular a sTREM-1 level, measured in a biological sample obtained from the subject upon hospitalization or upon admission in ICU. In one embodiment, the baseline level is a TREM-1 level, in particular a sTREM-1 level, measured after diagnosis of a disease caused by a coronavirus as described hereinabove, in particular after diagnosis in the hospital of a disease caused by a coronavirus as described hereinabove. In one embodiment, the baseline level is a TREM-1 level, in particular a sTREM-1 level, measured in a biological sample obtained from the subject before or at the beginning of the administration of a therapy, in particular before or at the beginning of the administration of a TREM-1 inhibitor as described herein.

According to one embodiment, the methods as described herein further comprise measuring the level of at least another biomarker in a biological sample from the subject as described hereinabove, and comparing the level of said at least another biomarker measured in the biological sample from the subject to a reference value as described herein.

In one embodiment, the methods as described herein further comprise measuring the level of interleukin-6 (IL-6) in a biological sample from the subject as described herein, and comparing the level of IL-6 measured in the biological sample from the subject to an IL-6 reference value.

IL-6 is a cytokine which has effects notably on inflammation, immune response, and hematopoiesis. Methods for measuring IL-6 levels are well-known to the skilled artisan and include, without being limited to, immunohistochemistry, multiplex methods, immunoassays, western blot, enzyme-linked immunosorbent assay (ELISA), sandwich ELISA, multiplex ELISA, capillary-based ELISA, electrochemiluminescence immunoassay (ECLIA), enzyme-linked fluorescent assay (ELFA), fluorescent-linked immunosorbent assay (FLISA), enzyme immunoassay (EIA), radioimmunoassay (RIA), flow cytometry (FACS), surface plasmon resonance (SPR), biolayer interferometry (BLI), immunochromatographic assay (ICA) and mass spectrometry-based approaches.

According to one embodiment, the IL-6 reference value is a reference IL-6 level, preferably a blood, plasma or serum level.

According to one embodiment, the reference IL-6 level is determined using an enzyme-linked immunosorbent assay (ELISA).

As mentioned hereinabove, it is well-known to the skilled artisan than the measure of the level of a protein may vary depending on the method or assay used to determine said measure. Therefore, according to one embodiment, the reference IL-6 level as determined using a given method or assay encompasses corresponding reference IL-6 levels as determined using another method or assay. In one embodiment, the reference IL-6 level as determined using an ELISA encompasses corresponding reference IL-6 levels as determined using another immunoassay, such as an electrochemiluminescence immunoassay (ECLIA) or an enzyme-linked fluorescence assay (ELFA). In one embodiment, the reference IL-6 level as determined using an ELISA encompasses the corresponding reference IL-6 level as determined using an ECLIA.

In one embodiment, the IL-6 reference value is derived from the measure of the IL-6 level in a biological sample obtained from one or more subject(s) who are substantially healthy as defined hereinabove. In one embodiment, the IL-6 reference value is derived from a reference population of subjects who are substantially healthy as defined hereinabove.

In one embodiment, the IL-6 reference value is derived from the measure of the IL-6 level in a biological sample obtained from one or more subjects diagnosed or identified as suffering, or having suffered, from a disease caused by a coronavirus, in particular from COVID-19 caused by SARS-CoV-2. In one embodiment, the IL-6 reference value is derived from a reference population of subjects diagnosed or identified as suffering, or having suffered, from a disease caused by a coronavirus, in particular from COVID-19 caused by SARS-CoV-2.

In one embodiment, the IL-6 reference value is an IL-6 level, preferably a blood, plasma or serum level, ranging from about 20 pg/mL to about 800 pg/mL, preferably from about 50 pg/mL to about 500 pg/mL, more preferably from about 100 pg/mL to about 400 pg/mL, even more preferably from about 200 pg/mL to about 300 pg/mL. In one embodiment, the IL-6 reference value is an IL-6 level, preferably a blood, plasma or serum level, of about 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 370, 375, 380, 385, 390, 395, or 400 pg/mL. In one embodiment, the IL-6 reference value is an IL-6 level, preferably a blood, plasma or serum level, of about 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 pg/mL.

In one embodiment, the IL-6 level, preferably a blood, plasma or serum level, as described above is determined using an ELISA, or is a corresponding IL-6 level, preferably a blood, plasma or serum level, as determined using another immunoassay, in particular an ECLIA.

One object of the invention is an in vitro method for identifying a subject suffering from a disease caused by a coronavirus as described hereinabove as being at risk of having or developing a severe form and/or a complication of the disease caused by a coronavirus, and/or at risk of death, said method comprising or consisting of:
measuring the level of TREM-1, in particular of sTREM-1, in a biological sample from the subject as described hereinabove; and
comparing the level of TREM-1, in particular of sTREM-1, measured in the biological sample from the subject to a reference value as described hereinabove.

In one embodiment, the present invention relates to an in vitro method for identifying a subject suffering from COVID-19 as being at risk of having or developing a severe form and/or a complication of COVID-19, and/or at risk of death, said method comprising or consisting of:
measuring the level of sTREM-1 in a biological sample from the subject as described hereinabove; and
comparing the level of sTREM-1 measured in the biological sample from the subject to a reference value as described hereinabove.

In one embodiment, a severe form of the disease caused by a coronavirus, in particular a severe form of COVID-19, is defined as requiring hospitalization. In one embodiment, a severe form of the disease caused by a coronavirus, in particular a severe form of COVID-19, is defined as requiring admission in ICU.

In one embodiment, a severe form of the disease caused by a coronavirus, in particular a severe form of COVID-19, is defined as requiring respiratory support as defined hereinabove. In one embodiment, the respiratory support is selected from the group comprising or consisting of supplemental oxygen (also called oxygen therapy) by mask or nasal prongs, positive pressure, high flow nasal oxygen; non-invasive ventilation (NIV); invasive mechanical ventilation (IMV) requiring tracheal intubation and/or tracheostomy; and extracorporeal membrane oxygenation (ECMO). In one embodiment, a severe form of the disease caused by a coronavirus, in particular a severe form of COVID-19, is defined as requiring invasive mechanical ventilation as described hereinabove. In one embodiment, a severe form of the disease caused by a coronavirus, in particular a severe form of COVID-19, is defined as requiring prolonged respiratory support, in particular prolonged invasive mechanical ventilation.

In one embodiment, prolonged respiratory support, in particular prolonged invasive mechanical ventilation, is respiratory support, in particular invasive mechanical ventilation, lasting at least 5, 6, 7, 8, 9, 10, 11, 12, 13, 14, 15, 16, 17, 18, 19, or 20 days, preferably at least 15 days. In one embodiment, prolonged respiratory support, in particular prolonged invasive mechanical ventilation, is a respiratory support, in particular invasive mechanical ventilation, lasting at least 1, 2, 3, 4, or 5 week(s), preferably at least 2 weeks. In one embodiment, prolonged respiratory support, in particular prolonged invasive mechanical ventilation, is a respiratory support, in particular invasive mechanical ventilation, lasting more than 5, 6, 7, 8, 9, 10, 11, 12, 13, 14, 15, 16, 17, 18, 19, or 20 days, preferably more than 15 days. In one embodiment, prolonged respiratory support, in particular prolonged invasive mechanical ventilation, is a respiratory support, in particular invasive mechanical ventilation, lasting more than 1, 2, 3, 4, or 5 week(s), preferably more than 2 weeks.

Examples of complications of a disease caused by a coronavirus, in particular of COVID-19, include, without being limited to, respiratory failure, including acute respiratory failure or acute respiratory distress syndrome (ARDS); respiratory failure requiring oxygen therapy (including non-invasive ventilation); respiratory failure requiring mechanical ventilation; persistence of respiratory failure including the requirement for prolonged mechanical ventilation, in particular prolonged mechanical ventilation lasting more than 15 days, and failed extubation; secondary infection or superinfection; thrombotic complications also referred to as thromboembolic complications or thromboembolic events, including venous and/or arterial thromboembolism, deep venous thrombosis, pulmonary embolism, and cerebrovascular accidents; cardiocirculatory failure (which may also be referred to as cardiovascular failure); renal failure including acute kidney injury (AKI); liver failure; and any combinations thereof.

In one embodiment, the one or more complication(s) of the disease caused by a coronavirus, in particular of COVID-19, is selected from the group comprising or consisting of, respiratory failure; persistence of respiratory failure; secondary infection or superinfection; thrombotic complications (also referred to as thromboembolic complications); cardiocirculatory failure (which may also be referred to as cardiovascular failure; renal failure; liver failure. In one embodiment, the complication of the disease caused by a coronavirus, in particular of COVID-19, is selected from the group comprising or consisting of respiratory failure, including acute respiratory failure or acute respiratory distress syndrome (ARDS); respiratory failure requiring oxygen therapy (including non-invasive ventilation); respiratory failure requiring mechanical ventilation; persistence of respiratory failure including the requirement for prolonged mechanical ventilation, in particular prolonged mechanical ventilation lasting more than 15 days, and failed extubation; secondary infection or superinfection; thrombotic complications also referred to as thromboembolic complications or thromboembolic events, including venous and/or arterial thromboembolism, deep venous thrombosis, pulmonary embolism, and cerebrovascular accidents; cardiocirculatory failure (which may also be referred to as cardiovascular failure); renal failure such as acute kidney injury (AKI); liver failure; and any combinations thereof.

In one embodiment, the complication of the disease caused by a coronavirus, in particular of COVID-19, is respiratory failure. In one embodiment, the complication of the disease caused by a coronavirus, in particular of COVID-19, is respiratory failure requiring oxygen therapy, respiratory failure requiring mechanical ventilation, acute respiratory failure and/or acute respiratory distress syndrome (ARDS). In one embodiment, the complication of the disease caused by a coronavirus, in particular of COVID-19, is persistence of respiratory failure, including the requirement for prolonged mechanical ventilation as defined hereinabove, and failed extubation.

In one embodiment, secondary infection is diagnosed when the subject shows clinical, laboratory or radiological signs or symptoms of pneumonia or bacteremia, optionally confirmed by positive culture.

In one embodiment, thrombotic complication (also referred to as thromboembolic complication or thromboembolic event) comprises or consists of venous and/or arterial thromboembolism, deep venous thrombosis, pulmonary embolism, and/or cerebrovascular accident.

In one embodiment, acute respiratory distress syndrome (ARDS) is diagnosed according to the Berlin Definition (ARDS Definition Task Force, Ranieri et al., JAMA. 2012; 307(23):2526-2533.).

In one embodiment, acute kidney injury is diagnosed according to the kidney disease improving global outcomes (KDIGO) clinical practice guidelines (Khwaja, Nephron Clin Pract. 2012; 120(4):c179-cl 84).

In one embodiment, cardiac failure is defined as the presence of one or more of the following: elevated serum levels of troponin, elevated serum levels of brain type natriuretic peptide (BNP), clinical or radiological features of cardiac failure, and/or a requirement for pharmacological or mechanical support of cardiac function.

In one embodiment, vascular dysfunction is defined as one or more of the following: clinical or laboratory features of vasodilatation, low systemic vascular resistance or blood pressure, and/or requirement for vasopressor medications to maintain adequate blood pressure.

In one embodiment, cardiocirculatory failure is diagnosed when the serum levels of troponin are greater than 12 pg/mL or when there is a requirement for vasopressors.

In one embodiment, the risk of death for the subject suffering from disease caused by a coronavirus, in particular COVID-19, is the risk of all-cause death. In one embodiment, the risk of death for the subject suffering from disease caused by a coronavirus, in particular COVID-19, is the risk of death from a symptom or a complication of the disease caused by a coronavirus, in particular COVID-19. In one embodiment, the risk of death for the subject suffering from disease caused by a coronavirus, in particular COVID-19, is the risk of death occurring after the coronavirus infection, in particular after the SARS-CoV-2 infection.

In one embodiment, a level of TREM-1, in particular of sTREM-1, measured in the biological sample from the subject higher than the reference value as described hereinabove is indicative of a risk of having or developing a severe form and/or a complication of the disease caused by a coronavirus, in particular COVID-19, and/or a risk of death.

In one embodiment, the reference value is derived from the measure of the TREM-1 level, in particular of the sTREM-1 level, in a biological sample obtained from one or more subjects who are substantially healthy as defined hereinabove. In one embodiment, the reference value is a reference sTREM-1 level, derived from a reference population of subjects who are substantially healthy.

In one embodiment, said reference value is a sTREM-1 level, preferably a blood, plasma or serum level, ranging from about 20 pg/mL to about 500 pg/mL, preferably from about 50 pg/mL to about 250 pg/mL, more preferably from about 100 pg/mL to about 200 pg/mL. In one embodiment, said reference value is a sTREM-1 level, preferably a blood, plasma or serum level, ranging from about 20 pg/mL to about 500 pg/mL, preferably from about 50 pg/mL to about 250 pg/mL, more preferably from about 100 pg/mL to about 200 pg/mL, as determined using an ELISA, or a corresponding TREM-1 level, in particular a corresponding sTREM-1 level, preferably a blood, plasma or serum level, as determined using another immunoassay, in particular an ECLIA. In one embodiment, said reference value is a sTREM-1 level, preferably a blood, plasma or serum level, of about 50, 75, 100, 125, 150, 175, 200, 225 or 250 pg/mL. In one embodiment, said reference value is a sTREM-1 level, preferably a blood, plasma or serum level, of about 50, 75, 100, 125, 150, 175, 200, 225 or 250 pg/mL as determined using an ELISA, or a corresponding TREM-1 level, in particular a corresponding sTREM-1 level, preferably a blood, plasma or serum level, as determined using another immunoassay, in particular an ECLIA.

In one embodiment, the reference value derived from a reference population of subjects who are substantially healthy is a sTREM-1 level, preferably a blood, plasma or serum level, ranging from about 50 pg/mL to about 500 pg/mL, preferably from about 200 pg/mL to about 450 pg/mL, more preferably from about 250 pg/mL to about 400 pg/mL, as determined using an ECLIA, or a corresponding TREM-1 level, in particular a corresponding sTREM-1 level, preferably a blood, plasma or serum level, as determined using another immunoassay, in particular an ELISA.

In one embodiment, the reference value derived from a reference population of subjects who are substantially healthy is a TREM-1 level, in particular a sTREM-1 level, preferably a blood, plasma or serum level, of about 50, 55, 60, 65, 70, 75, 80, 85, 90, 95, 100, 105, 110, 115, 120, 125, 130, 135, 140, 145, or 150 pg/mL. In one embodiment, the reference value derived from a reference population of subjects who are substantially healthy is a TREM-1 level, in particular a sTREM-1 level, preferably a blood, plasma or serum level, of about 50, 55, 60, 65, 70, 75, 80, 85, 90, 95, 100, 105, 110, 115, 120, 125, 130, 135, 140, 145, or 150 pg/mL as determined using an ELISA, or a corresponding TREM-1 level, in particular a corresponding sTREM-1 level, preferably a blood, plasma or serum level, as determined using another immunoassay, in particular an ECLIA. In one embodiment, the reference value derived from a reference population of subjects who are substantially healthy is a TREM-1 level, in particular a sTREM-1 level, preferably a blood, plasma or serum level, of about 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 pg/mL. In one embodiment, the reference value derived from a reference population of subjects who are substantially healthy is a TREM-1 level, in particular a sTREM-1 level, preferably a blood, plasma or serum level, of about 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 pg/mL as determined using an ELISA, or a corresponding TREM-1 level, in particular a corresponding sTREM-1 level, preferably a blood, plasma or serum level, as determined using another immunoassay, in particular an ECLIA.

In one embodiment, the reference value derived from a reference population of subjects who are substantially healthy is a TREM-1 level, in particular a sTREM-1 level, preferably a blood, plasma or serum level, of about 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355; 360, 365, 370, 375, 380, 385, 390, 395, 400, 405, 410, or 415 pg/mL as determined using an ECLIA, or a corresponding TREM-1 level, in particular a corresponding sTREM-1 level, preferably a blood, plasma or serum level, as determined using another immunoassay, in particular an ELISA.

In one embodiment, the reference value is derived from the measure of the TREM-1 level, in particular of the sTREM-1 level, in a biological sample obtained from one or more subjects diagnosed or identified as suffering, or having suffered, from a disease caused by a coronavirus, in particular from COVID-19 caused by SARS-CoV-2. In one embodiment, the reference value is a reference sTREM-1 level, derived from a reference population of subjects diagnosed or identified as suffering, or having suffered, from a disease caused by a coronavirus, in particular from COVID-19 caused by SARS-CoV-2.

In one embodiment, said reference value is a TREM-1 level, in particular a sTREM-1 level, preferably a blood, plasma or serum level, ranging from about 50 pg/mL to about 800 pg/mL, preferably from about 75 pg/mL to about 600 pg/mL, more preferably from about 100 pg/mL to about 400 pg/mL, even more preferably from about 130 pg/mL to about 400 pg/mL. In one embodiment, said reference value is a TREM-1 level, in particular a sTREM-1 level, preferably a blood, plasma or serum level, ranging from about 50 pg/mL to about 800 pg/mL, preferably from about 75 pg/mL to about 600 pg/mL, more preferably from about 100 pg/mL to about 400 pg/mL, even more preferably from about 130 pg/mL to about 400 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value, preferably the reference value derived from a reference population as described hereinabove, is a TREM-1 level, in particular a sTREM-1 level, preferably a blood, plasma or serum level, ranging from about 130 pg/mL to about 600 pg/mL, preferably from about 200 pg/mL to about 500 pg/mL, more preferably from about 250 pg/mL to about 400 pg/mL, even more preferably from about 300 pg/mL to about 375 pg/mL. In one embodiment, the reference value, preferably the reference value derived from a reference population as described hereinabove, is a TREM-1 level, in particular a sTREM-1 level, preferably a blood, plasma or serum level, ranging from about 130 pg/mL to about 600 pg/mL, preferably from about 200 pg/mL to about 500 pg/mL, more preferably from about 250 pg/mL to about 400 pg/mL, even more preferably from about 300 pg/mL to about 375 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value, preferably the reference value derived from a reference population as described hereinabove, is a TREM-1 level, in particular a sTREM-1 level, preferably a blood, plasma or serum level, ranging from about 350 pg/mL to about 1200 pg/mL, preferably from about 600 pg/mL to about 1100 pg/mL, more preferably from about 700 pg/mL to about 1000 pg/mL, as determined using an ECLIA, or a corresponding TREM-1 level, in particular a corresponding sTREM-1 level, preferably a blood, plasma or serum level, as determined using another immunoassay, in particular an ELISA.

In one embodiment, the reference value, preferably the reference value derived from a reference population as described hereinabove, is a TREM-1 level, in particular a sTREM-1 level, preferably a blood, plasma or serum level, of about 75, 100, 125, 150, 175, 200, 225, 250, 275, 300, 325, 350, 375 or 400 pg/mL. In one embodiment, the reference value, preferably the reference value derived from a reference population as described hereinabove, is a TREM-1 level, in particular a sTREM-1 level, preferably a blood, plasma or serum level, of about 75, 100, 125, 150, 175, 200, 225, 250, 275, 300, 325, 350, 375 or 400 pg/mL, as determined using an ELISA, or a corresponding TREM-1 level, in particular a corresponding sTREM-1 level, preferably a blood, plasma or serum level, as determined using another immunoassay, in particular an ECLIA. In one embodiment, the reference value, preferably the reference value derived from a reference population as described hereinabove, is a TREM-1 level, in particular a sTREM-1 level, preferably a blood, plasma or serum level, of about 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, or 430 pg/mL. In one embodiment, the reference value, preferably the reference value derived from a reference population as described hereinabove, is a TREM-1 level, in particular a sTREM-1 level, preferably a blood, plasma or serum level, of about 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, or 430 pg/mL, as determined using an ELISA, or a corresponding TREM-1 level, in particular a corresponding sTREM-1 level, preferably a blood, plasma or serum level, as determined using another immunoassay, in particular an ECLIA. In one embodiment, the reference value, preferably the reference value derived from a reference population as described hereinabove, is a TREM-1 level, in particular a sTREM-1 level, preferably a blood, plasma or serum level, of about 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 pg/mL. In one embodiment, the reference value, preferably the reference value derived from a reference population as described hereinabove, is a TREM-1 level, in particular a sTREM-1 level, preferably a blood, plasma or serum level, of about 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value, preferably the reference value derived from a reference population as described hereinabove, is a TREM-1 level, in particular a sTREM-1 level, preferably a blood, plasma or serum level, of about 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 885, 890, 895, 900, 905, 910, 915, 920, 925, 930, 935, 940, 945, 950, 955, 960, 965, 970, 975, 980, 985, 990, 995, or 1000 pg/mL as determined using an ECLIA, or a corresponding TREM-1 level, in particular a corresponding sTREM-1 level, preferably a blood, plasma or serum level, as determined using another immunoassay, in particular an ELISA.

According to one embodiment, a level of TREM-1, in particular of sTREM-1, measured in the biological sample from the subject higher than the reference value is indicative of a risk of having or developing a severe form and/or a complication of the disease caused by a coronavirus, in particular COVID-19. In one embodiment, said reference value is a TREM-1 level, in particular a sTREM-1 level, preferably a blood, plasma or serum level, ranging from about 100 pg/mL to about 400 pg/mL, preferably from about 130 pg/mL to about 300 pg/mL, more preferably from about 130 pg/mL to about 200 pg/mL, even more preferably from about 135 pg/mL to about 190 pg/mL, preferably as determined using an ELISA, or a corresponding TREM-1 level, in particular a corresponding sTREM-1 level, preferably a blood, plasma or serum level, as determined using another immunoassay, in particular an ECLIA. In one embodiment, said reference value is a TREM-1 level, in particular a sTREM-1 level, preferably a blood, plasma or serum level, of about 130, 135, 140, 145, 150, 155, 160, 165, 170, 175, 180, 185, 190, 195, 200, 205, 210, 215, 220, 225, 230, 235, 240, 245, or 250 pg/mL, as determined using an ELISA, or a corresponding TREM-1 level, in particular a corresponding sTREM-1 level, preferably a blood, plasma or serum level, as determined using another immunoassay, in particular an ECLIA. In one embodiment, said reference value is a TREM-1 level, in particular a sTREM-1 level, preferably a blood, plasma or serum level, of about 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 181, 182, 183, 184, 185, 186, 187, 188, 189, 190,191, 192, 193, 194, 195, 196, 197, 198, 199, or 200 pg/mL, preferably as determined using an ELISA, or a corresponding TREM-1 level, in particular a corresponding sTREM-1 level, preferably a blood, plasma or serum level, as determined using another immunoassay, in particular an ECLIA.

According to one embodiment, a level of TREM-1, in particular of sTREM-1, measured in the biological sample from the subject higher than the reference value is indicative of a risk of death. In one embodiment, said reference value is a TREM-1 level, in particular a sTREM-1 level, preferably a blood, plasma or serum level, ranging from about 200 pg/mL to about 500 pg/mL, preferably from about 250 pg/mL to about 400 pg/mL, more preferably from about 300 pg/mL to about 375 pg/mL, preferably as determined using an ELISA, or a corresponding TREM-1 level, in particular a corresponding sTREM-1 level, preferably a blood, plasma or serum level, as determined using another immunoassay, in particular an ECLIA. In one embodiment, said reference value is a TREM-1 level, in particular a sTREM-1 level, preferably a blood, plasma or serum level, of about 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, or 430 pg/mL, preferably as determined using an ELISA, or a corresponding TREM-1 level, in particular a corresponding sTREM-1 level, preferably a blood, plasma or serum level, as determined using another immunoassay, in particular an ECLIA. In one embodiment, said reference value is a TREM-1 level, in particular a sTREM-1 level, preferably a blood, plasma or serum level, of about 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375 pg/mL, preferably as determined using an ELISA, or a corresponding TREM-1 level, in particular a corresponding sTREM-1 level, preferably a blood, plasma or serum level, as determined using another immunoassay, in particular an ECLIA.

According to one embodiment, the method further comprises measuring the level of interleukin-6 (IL-6) in a biological sample from the subject as described hereinabove, and comparing the level of IL-6 measured in the biological sample from the subject to an IL-6 reference value as described hereinabove.

In one embodiment, a level of IL-6 measured in the biological sample from the subject higher than the IL-6 reference value as described hereinabove is indicative of a risk of having or developing a severe form and/or a complication of the disease caused by a coronavirus, in particular COVID-19, and/or a risk of death.

In one embodiment, the present invention relates to an in vitro method for identifying a subject suffering from a disease caused by a coronavirus, in particular COVID-19, as being at risk of having or developing a severe form and/or a complication of the disease caused by a coronavirus, in particular COVID-19, and/or at risk of death, said method comprising or consisting of:
measuring the level of sTREM-1 in a biological sample from the subject as described hereinabove; and
comparing the level of sTREM-1 measured in the biological sample from the subject to a reference value, preferably a reference sTREM-1 level derived from a reference population of subjects who are substantially healthy as described hereinabove, or derived from a reference population of subjects diagnosed or identified as suffering, or having suffered, from a disease caused by a coronavirus, in particular from COVID-19 caused by SARS-CoV-2, as described hereinabove,
   wherein a level of sTREM-1 measured in the biological sample from the subject higher than the reference value is indicative of a risk of having or developing a severe form and/or a complication of the disease caused by a coronavirus, in particular COVID-19, or a risk of death.

In one embodiment, the present invention relates to an in vitro method for identifying a subject suffering from a disease caused by a coronavirus, in particular COVID-19, as being at risk of having or developing a severe form and/or a complication of the disease caused by a coronavirus, in particular COVID-19, and/or at risk of death, said method comprising or consisting of:
measuring the level of sTREM-1 in a biological sample from the subject as described hereinabove, and measuring the level of IL-6 in a biological sample from the subject as described hereinabove; and
comparing the level of sTREM-1 measured in the biological sample from the subject to a sTREM-1 reference level as described hereinabove, and comparing the level of IL-6 measured in the biological sample from the subject to an IL-6 reference level as described hereinabove,
   wherein a level of sTREM-1 measured in the biological sample from the subject higher than the sTREM-1 reference level and a level of IL-6 measured in the biological sample from the subject higher than the IL-6 reference level are indicative of a risk of having or developing a severe form and/or a complication of the disease caused by a coronavirus, in particular COVID-19, and/or a risk of death.

Another object of the invention is an in vitro method for determining the prognosis of a subject suffering from a disease caused by a coronavirus as described hereinabove, said method comprising or consisting of:
measuring the level of TREM-1, in particular of sTREM-1, in a biological sample from the subject as described hereinabove; and
comparing the level of TREM-1, in particular of sTREM-1, measured in the biological sample from the subject to a reference value as described hereinabove.

In one embodiment, the present invention relates to an in vitro method for determining the prognosis of a subject suffering from COVID-19, said method comprising or consisting of:
measuring the level of sTREM-1 in a biological sample from the subject as described hereinabove; and
comparing the level of sTREM-1 measured in the biological sample from the subject to a reference value as described hereinabove.

In one embodiment, a level of TREM-1, in particular of sTREM-1, measured in the biological sample from the subject higher than the reference value as described hereinabove is indicative of a poor prognosis for the subject suffering from a disease caused by a coronavirus as described hereinabove, in particular COVID-19.

In one embodiment, a poor prognosis is defined as an elevated risk of developing a complication and/or an elevated risk of death.

In one embodiment, the reference value is derived from the measure of the TREM-1 level, in particular of the sTREM-1 level, in a biological sample obtained from one or more subjects who are substantially healthy as defined hereinabove. In one embodiment, the reference value is a reference sTREM-1 level, derived from a reference population of subjects who are substantially healthy.

In one embodiment, said reference value is a sTREM-1 level, preferably a blood, plasma or serum level, ranging from about 20 pg/mL to about 500 pg/mL, preferably from about 50 pg/mL to about 250 pg/mL, more preferably from about 100 pg/mL to about 200 pg/mL. In one embodiment, said reference value is a sTREM-1 level, preferably a blood, plasma or serum level, ranging from about 20 pg/mL to about 500 pg/mL, preferably from about 50 pg/mL to about 250 pg/mL, more preferably from about 100 pg/mL to about 200 pg/mL, as determined using an ELISA, or a corresponding TREM-1 level, in particular a corresponding sTREM-1 level, preferably a blood, plasma or serum level, as determined using another immunoassay, in particular an ECLIA. In one embodiment, said reference value is a sTREM-1 level, preferably a blood, plasma or serum level, of about 50, 75, 100, 125, 150, 175, 200, 225 or 250 pg/mL. In one embodiment, said reference value is a sTREM-1 level, preferably a blood, plasma or serum level, of about 50, 75, 100, 125, 150, 175, 200, 225 or 250 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value derived from a reference population of subjects who are substantially healthy is a TREM-1 level, in particular a sTREM-1 level, preferably a blood, plasma or serum level, ranging from about 50 pg/mL to about 500 pg/mL, preferably from about 200 pg/mL to about 450 pg/mL, more preferably from about 250 pg/mL to about 400 pg/mL, as determined using an ECLIA, or a corresponding TREM-1 level, in particular a corresponding sTREM-1 level, preferably a blood, plasma or serum level, as determined using another immunoassay, in particular an ELISA

In one embodiment, the reference value derived from a reference population of subjects who are substantially healthy is a TREM-1 level, in particular a sTREM-1 level, preferably a blood, plasma or serum level, of about 50, 55, 60, 65, 70, 75, 80, 85, 90, 95, 100, 105, 110, 115, 120, 125, 130, 135, 140, 145, or 150 pg/mL. In one embodiment, the reference value derived from a reference population of subjects who are substantially healthy is a TREM-1 level, in particular a sTREM-1 level, preferably a blood, plasma or serum level, of about 50, 55, 60, 65, 70, 75, 80, 85, 90, 95, 100, 105, 110, 115, 120, 125, 130, 135, 140, 145, or 150 pg/mL, as determined using an ELISA, or a corresponding TREM-1 level, in particular a corresponding sTREM-1 level, preferably a blood, plasma or serum level, as determined using another immunoassay, in particular an ECLIA. In one embodiment, the reference value derived from a reference population of subjects who are substantially healthy is a TREM-1 level, in particular a sTREM-1 level, preferably a blood, plasma or serum level, of about 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 pg/mL. In one embodiment, the reference value derived from a reference population of subjects who are substantially healthy is a TREM-1 level, in particular a sTREM-1 level, preferably a blood, plasma or serum level, of about 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value derived from a reference population of subjects who are substantially healthy is a TREM-1 level, in particular a sTREM-1 level, preferably a blood, plasma or serum level, of about 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355; 360, 365, 370, 375, 380, 385, 390, 395, 400, 405, 410, or 415 pg/mL as determined using an ECLIA, or a corresponding TREM-1 level, in particular a corresponding sTREM-1 level, preferably a blood, plasma or serum level, as determined using another immunoassay, in particular an ELISA.

In one embodiment, the reference value is derived from the measure of the TREM-1 level, in particular of sTREM-1 level in a biological sample from one or more subjects diagnosed or identified as suffering, or having suffered, from a disease caused by a coronavirus, in particular from COVID-19. In one embodiment, the reference value is a reference sTREM-1 level derived from a reference population of subjects diagnosed or identified as suffering, or having suffered, from a disease caused by a coronavirus, in particular from COVID-19.

In one embodiment, said reference value is a TREM-1 level, in particular a sTREM-1 level, preferably a blood, plasma or serum level, ranging from about 50 pg/mL to about 800 pg/mL, preferably from about 75 pg/mL to about 600 pg/mL, more preferably from about 100 pg/mL to about 400 pg/mL, even more preferably from about 130 pg/mL to about 400 pg/mL. In one embodiment, said reference value is a TREM-1 level, in particular a sTREM-1 level, preferably a blood, plasma or serum level, ranging from about 50 pg/mL to about 800 pg/mL, preferably from about 75 pg/mL to about 600 pg/mL, more preferably from about 100 pg/mL to about 400 pg/mL, even more preferably from about 130 pg/mL to about 400 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value, preferably the reference value derived from a reference population as described hereinabove, is a TREM-1 level, in particular a sTREM-1 level, preferably a blood, plasma or serum level, ranging from about 130 pg/mL to about 600 pg/mL, preferably from about 200 pg/mL to about 500 pg/mL, more preferably from about 250 pg/mL to about 400 pg/mL, even more preferably from about 300 pg/mL to about 375 pg/mL. In one embodiment, the reference value, preferably the reference value derived from a reference population as described hereinabove, is a TREM-1 level, in particular a sTREM-1 level, preferably a blood, plasma or serum level, ranging from about 130 pg/mL to about 600 pg/mL, preferably from about 200 pg/mL to about 500 pg/mL, more preferably from about 250 pg/mL to about 400 pg/mL, even more preferably from about 300 pg/mL to about 375 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value, preferably the reference value derived from a reference population as described hereinabove, is a TREM-1 level, in particular a sTREM-1 level, preferably a blood, plasma or serum level, ranging from about 350 pg/mL to about 1200 pg/mL, preferably from about 600 pg/mL to about 1100 pg/mL, more preferably from about 700 pg/mL to about 1000 pg/mL, as determined using an ECLIA, or a corresponding TREM-1 level, in particular a corresponding sTREM-1 level, preferably a blood, plasma or serum level, as determined using another immunoassay, in particular an ELISA.

In one embodiment, said reference value is a TREM-1 level, in particular a sTREM-1 level, preferably a blood, plasma or serum level, of about 75, 100, 125, 150, 175, 200, 225, 250, 275, 300, 325, 350, 375 or 400 pg/mL. In one embodiment, said reference value is a TREM-1 level, in particular a sTREM-1 level, preferably a blood, plasma or serum level, of about 75, 100, 125, 150, 175, 200, 225, 250, 275, 300, 325, 350, 375 or 400 pg/mL. In one embodiment, the reference value, preferably the reference value derived from a reference population as described hereinabove, is a TREM-1 level, in particular a sTREM-1 level, preferably a blood, plasma or serum level, of about 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, or 430 pg/mL. In one embodiment, the reference value, preferably the reference value derived from a reference population as described hereinabove, is a TREM-1 level, in particular a sTREM-1 level, preferably a blood, plasma or serum level, of about 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, or 430 pg/mL. In one embodiment, the reference value, preferably the reference value derived from a reference population as described hereinabove, is a TREM-1 level, in particular a sTREM-1 level, preferably a blood, plasma or serum level, of about 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 pg/mL. In one embodiment, the reference value, preferably the reference value derived from a reference population as described hereinabove, is a TREM-1 level, in particular a sTREM-1 level, preferably a blood, plasma or serum level, of about 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, or 430 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value, preferably the reference value derived from a reference population as described hereinabove, is a TREM-1 level, in particular a sTREM-1 level, preferably a blood, plasma or serum level, of about 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 885, 890, 895, 900, 905, 910, 915, 920, 925, 930, 935, 940, 945, 950, 955, 960, 965, 970, 975, 980, 985, 990, 995, or 1000 pg/mL as determined using an ECLIA, or a corresponding TREM-1 level, in particular a corresponding sTREM-1 level, preferably a blood, plasma or serum level, as determined using another immunoassay, in particular an ELISA.

According to one embodiment, a level of TREM-1, in particular of sTREM-1, measured in the biological sample from the subject higher than the reference value is indicative of a poor prognosis being defined as an elevated risk of developing a complication. In one embodiment, said reference value is a TREM-1 level, in particular a sTREM-1 level, preferably a blood, plasma or serum level, ranging from about 100 pg/mL to about 400 pg/mL, preferably from about 130 pg/mL to about 300 pg/mL, more preferably from about 130 pg/mL to about 200 pg/mL, even more preferably from about 135 pg/mL to about 190 pg/mL, preferably as determined using an ELISA, or a corresponding TREM-1 level, in particular a corresponding sTREM-1 level, preferably a blood, plasma or serum level, as determined using another immunoassay, in particular an ECLIA. In one embodiment, said reference value is a TREM-1 level, in particular a sTREM-1 level, preferably a blood, plasma or serum level, of about 130, 135, 140, 145, 150, 155, 160, 165, 170, 175, 180, 185, 190, 195, 200, 205, 210, 215, 220, 225, 230, 235, 240, 245, or 250 pg/mL, preferably as determined using an ELISA, or a corresponding TREM-1 level, in particular a corresponding sTREM-1 level, preferably a blood, plasma or serum level, as determined using another immunoassay, in particular an ECLIA. In one embodiment, said reference value is a TREM-1 level, in particular a sTREM-1 level, preferably a blood, plasma or serum level, of about 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 181, 182, 183, 184, 185, 186, 187, 188, 189, 190,191, 192, 193, 194, 195, 196, 197, 198, 199, or 200 pg/mL, preferably as determined using an ELISA, or a corresponding TREM-1 level, in particular a corresponding sTREM-1 level, preferably a blood, plasma or serum level, as determined using another immunoassay, in particular an ECLIA.

According to one embodiment, a level of TREM-1, in particular of sTREM-1, measured in the biological sample from the subject higher than the reference value is indicative of a poor prognosis being defined as an elevated risk of death. In one embodiment, said reference value is a TREM-1 level, in particular a sTREM-1 level, preferably a blood, plasma or serum level, ranging from about 200 pg/mL to about 500 pg/mL, preferably from about 250 pg/mL to about 400 pg/mL, more preferably from about 300 pg/mL to about 375 pg/mL, preferably as determined using an ELISA, or a corresponding TREM-1 level, in particular a corresponding sTREM-1 level, preferably a blood, plasma or serum level, as determined using another immunoassay, in particular an ECLIA. In one embodiment, said reference value is a TREM-1 level, in particular a sTREM-1 level, preferably a blood, plasma or serum level, of about 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, or 430 pg/mL, preferably as determined using an ELISA, or a corresponding TREM-1 level, in particular a corresponding sTREM-1 level, preferably a blood, plasma or serum level, as determined using another immunoassay, in particular an ECLIA. In one embodiment, said reference value is a TREM-1 level, in particular a sTREM-1 level, preferably a blood, plasma or serum level, of about 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375 pg/mL, preferably as determined using an ELISA, or a corresponding TREM-1 level, in particular a corresponding sTREM-1 level, preferably a blood, plasma or serum level, as determined using another immunoassay, in particular an ECLIA.

According to one embodiment, the method of the invention comprises measuring the level of TREM-1, in particular of sTREM-1, in a biological sample from the subject as described hereinabove on at least two occasions. In other words, in one embodiment, the method of the invention comprises measuring the level of TREM-1, in particular of sTREM-1, in a biological sample from the subject as described hereinabove several times over time. Thus, it is to be understood that, in one embodiment, the method of the invention comprises measuring the level of TREM-1, in particular of sTREM-1, in several biological samples obtained from the subject as described hereinabove over time.

In one embodiment, the at least two measures of the level of TREM-1, in particular of sTREM-1, in a biological sample from the subject are separated by at least 24 h, 36 h, 48 h, 60 h, or 72 h. In one embodiment, the at least two measures of the level of TREM-1, in particular of sTREM-1, in a biological sample from the subject are separated by at least 1, 2, or 3 days.

In one embodiment, the method of the invention comprises measuring the level of TREM-1, in particular of sTREM-1, in a biological sample from the subject as described hereinabove every 24 h, every 36 h, every 48 h, every 60 h or every 72 h. In one embodiment, the method of the invention comprises measuring the level of TREM-1, in particular of sTREM-1, in a biological sample from the subject as described hereinabove every day, every 2 days or every 3 days.

In one embodiment, a level of TREM-1, in particular of sTREM-1, measured in the biological sample from the subject which is higher than the reference value as described hereinabove and which remains stable or increases over time is indicative of a poor prognosis for the subject suffering from a disease caused by a coronavirus as described hereinabove, in particular COVID-19.

In one embodiment, a level of TREM-1, in particular of sTREM-1, measured in the biological sample from the subject which remains stable over time is a level of TREM-1, in particular of sTREM-1, measured in the biological sample from the subject which remains stable over at least 1, 2, 3, 4, 5, 6, 7, 8, 9, 10, 11, 12, 13, 14, 15, 16, 17, 18, 19, 20 or 21 days. In one embodiment, a level of TREM-1, in particular of sTREM-1, measured in the biological sample from the subject which increases over time is a level of TREM-1, in particular of sTREM-1, measured in the biological sample from the subject which increases over at least 1, 2, 3, 4, 5, 6, 7, 8, 9, 10, 11, or 12 days.

In one embodiment, the present invention relates to an in vitro method for determining the prognosis of a subject suffering from a disease caused by a coronavirus, in particular COVID-19, said method comprising or consisting of:
measuring the level of sTREM-1 in a biological sample from the subject as described hereinabove; and
comparing the level of sTREM-1 measured in the biological sample from the subject to a reference value, preferably a reference sTREM-1 level derived from a reference population of subjects who are substantially healthy as described hereinabove, or derived from a reference population of subjects diagnosed or identified as suffering, or having suffered, from a disease caused by a coronavirus, in particular from COVID-19 caused by SARS-CoV-2, as described hereinabove,
   wherein a level of sTREM-1 measured in the biological sample from the subject higher than the reference value, preferably a level of sTREM-1 measured in the biological sample from the subject which is higher than the reference value as described hereinabove and which remains stable or increases over time, is indicative of a poor prognosis for the subject suffering from a disease caused by a coronavirus as described hereinabove, in particular COVID-19.

Another object of the invention is an in vitro method for determining the severity of a disease caused by a coronavirus as described hereinabove in a subject, said method comprising or consisting of:
measuring the level of TREM-1, in particular of sTREM-1, in a biological sample from the subject as described hereinabove; and
comparing the level of TREM-1, in particular of sTREM-1, measured in the biological sample from the subject to a reference value.

In one embodiment, the present invention relates to an in vitro method for determining the severity of COVID-19 in a subject, said method comprising or consisting of:
measuring the level of sTREM-1 in a biological sample from the subject as described hereinabove; and
comparing the level of sTREM-1 measured in the biological sample from the subject to a reference value as described hereinabove.

In one embodiment, the level of TREM-1, in particular of sTREM-1, measured in the biological sample from the subject is correlated with the severity of the disease caused by a coronavirus, in particular COVID-19, in said subject.

In one embodiment, the higher the level of TREM-1, in particular of sTREM-1, measured in the biological sample from the subject is as compared to the reference value as described hereinabove, the more severe the disease caused by a coronavirus, in particular COVID-19, is in said subject.

In one embodiment, the reference value is derived from the measure of the TREM-1 level, in particular of the sTREM-1 level, in a biological sample obtained from one or more subjects who are substantially healthy as defined hereinabove. In one embodiment, the reference value is a reference sTREM-1 level derived from a reference population of subjects who are substantially healthy.

In one embodiment, said reference value is a sTREM-1 level, preferably a blood, plasma or serum level, ranging from about 20 pg/mL to about 500 pg/mL, preferably from about 50 pg/mL to about 250 pg/mL, more preferably from about 100 pg/mL to about 200 pg/mL. In one embodiment, said reference value is a sTREM-1 level, preferably a blood, plasma or serum level, ranging from about 20 pg/mL to about 500 pg/mL, preferably from about 50 pg/mL to about 250 pg/mL, more preferably from about 100 pg/mL to about 200 pg/mL, as determined using an ELISA, or a corresponding TREM-1 level, in particular a corresponding sTREM-1 level, preferably a blood, plasma or serum level, as determined using another immunoassay, in particular an ECLIA. In one embodiment, said reference value is a sTREM-1 level, preferably a blood, plasma or serum level, of about 50, 75, 100, 125, 150, 175, 200, 225 or 250 pg/mL. In one embodiment, said reference value is a sTREM-1 level, preferably a blood, plasma or serum level, of about 50, 75, 100, 125, 150, 175, 200, 225 or 250 pg/mL as determined using an ELISA, or a corresponding TREM-1 level, in particular a corresponding sTREM-1 level, preferably a blood, plasma or serum level, as determined using another immunoassay, in particular an ECLIA.

In one embodiment, the reference value derived from a reference population of subjects who are substantially healthy is a TREM-1 level, in particular a sTREM-1 level, preferably a blood, plasma or serum level, ranging from about 50 pg/mL to about 500 pg/mL, preferably from about 200 pg/mL to about 450 pg/mL, more preferably from about 250 pg/mL to about 400 pg/mL, as determined using an ECLIA, or a corresponding TREM-1 level, in particular a corresponding sTREM-1 level, preferably a blood, plasma or serum level, as determined using another immunoassay, in particular an ELISA.

In one embodiment, the reference value derived from a reference population of subjects who are substantially healthy is a TREM-1 level, in particular a sTREM-1 level, preferably a blood, plasma or serum level, of about 50, 55, 60, 65, 70, 75, 80, 85, 90, 95, 100, 105, 110, 115, 120, 125, 130, 135, 140, 145, or 150 pg/mL. In one embodiment, the reference value derived from a reference population of subjects who are substantially healthy is a TREM-1 level, in particular a sTREM-1 level, preferably a blood, plasma or serum level, of about 50, 55, 60, 65, 70, 75, 80, 85, 90, 95, 100, 105, 110, 115, 120, 125, 130, 135, 140, 145, or 150 pg/mL as determined using an ELISA, or a corresponding TREM-1 level, in particular a corresponding sTREM-1 level, preferably a blood, plasma or serum level, as determined using another immunoassay, in particular an ECLIA. In one embodiment, the reference value derived from a reference population of subjects who are substantially healthy is a TREM-1 level, in particular a sTREM-1 level, preferably a blood, plasma or serum level, of about 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 pg/mL. In one embodiment, the reference value derived from a reference population of subjects who are substantially healthy is a TREM-1 level, in particular a sTREM-1 level, preferably a blood, plasma or serum level, of about 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 pg/mL as determined using an ELISA, or a corresponding TREM-1 level, in particular a corresponding sTREM-1 level, preferably a blood, plasma or serum level, as determined using another immunoassay, in particular an ECLIA.

In one embodiment, the reference value derived from a reference population of subjects who are substantially healthy is a TREM-1 level, in particular a sTREM-1 level, preferably a blood, plasma or serum level, of about 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355; 360, 365, 370, 375, 380, 385, 390, 395, 400, 405, 410, or 415 pg/mL as determined using an ECLIA, or a corresponding TREM-1 level, in particular a corresponding sTREM-1 level, preferably a blood, plasma or serum level, as determined using another immunoassay, in particular an ELISA.

In one embodiment, the reference value is derived from the measure of the TREM-1 level, in particular of sTREM-1 level in a biological sample from one or more subjects diagnosed or identified as suffering, or having suffered, from a disease caused by a coronavirus, in particular from COVID-19. In one embodiment, the reference value is a reference sTREM-1 level derived from a reference population of subjects diagnosed or identified as suffering, or having suffered, from a disease caused by a coronavirus, in particular from COVID-19.

In one embodiment, said reference value is a TREM-1 level, in particular a sTREM-1 level, preferably a blood, plasma or serum level, ranging from about 50 pg/mL to about 800 pg/mL, preferably from about 75 pg/mL to about 600 pg/mL, more preferably from about 100 pg/mL to about 400 pg/mL, even more preferably from about 130 pg/mL to about 400 pg/mL. In one embodiment, said reference value is a TREM-1 level, in particular a sTREM-1 level, preferably a blood, plasma or serum level, ranging from about 50 pg/mL to about 800 pg/mL, preferably from about 75 pg/mL to about 600 pg/mL, more preferably from about 100 pg/mL to about 400 pg/mL, even more preferably from about 130 pg/mL to about 400 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value, preferably the reference value derived from a reference population as described hereinabove, is a TREM-1 level, in particular a sTREM-1 level, preferably a blood, plasma or serum level, ranging from about 130 pg/mL to about 600 pg/mL, preferably from about 200 pg/mL to about 500 pg/mL, more preferably from about 250 pg/mL to about 400 pg/mL, even more preferably from about 300 pg/mL to about 375 pg/mL. In one embodiment, the reference value, preferably the reference value derived from a reference population as described hereinabove, is a TREM-1 level, in particular a sTREM-1 level, preferably a blood, plasma or serum level, ranging from about 130 pg/mL to about 600 pg/mL, preferably from about 200 pg/mL to about 500 pg/mL, more preferably from about 250 pg/mL to about 400 pg/mL, even more preferably from about 300 pg/mL to about 375 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value, preferably the reference value derived from a reference population as described hereinabove, is a TREM-1 level, in particular a sTREM-1 level, preferably a blood, plasma or serum level, ranging from about 350 pg/mL to about 1200 pg/mL, preferably from about 600 pg/mL to about 1100 pg/mL, more preferably from about 700 pg/mL to about 1000 pg/mL, as determined using an ECLIA, or a corresponding TREM-1 level, in particular a corresponding sTREM-1 level, preferably a blood, plasma or serum level, as determined using another immunoassay, in particular an ELISA.

In one embodiment, said reference value is a TREM-1 level, in particular a sTREM-1 level, preferably a blood, plasma or serum level, of about 75, 100, 125, 150, 175, 200, 225, 250, 275, 300, 325, 350, 375 or 400 pg/mL. In one embodiment, said reference value is a TREM-1 level, in particular a sTREM-1 level, preferably a blood, plasma or serum level, of about 75, 100, 125, 150, 175, 200, 225, 250, 275, 300, 325, 350, 375 or 400 pg/mL as determined using an ELISA, or a corresponding TREM-1 level, in particular a corresponding sTREM-1 level, preferably a blood, plasma or serum level, as determined using another immunoassay, in particular an ECLIA. In one embodiment, the reference value, preferably the reference value derived from a reference population as described hereinabove, is a TREM-1 level, in particular a sTREM-1 level, preferably a blood, plasma or serum level, of about 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, or 430 pg/mL. In one embodiment, the reference value, preferably the reference value derived from a reference population as described hereinabove, is a TREM-1 level, in particular a sTREM-1 level, preferably a blood, plasma or serum level, of about 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, or 430 pg/mL as determined using an ELISA, or a corresponding TREM-1 level, in particular a corresponding sTREM-1 level, preferably a blood, plasma or serum level, as determined using another immunoassay, in particular an ECLIA. In one embodiment, the reference value, preferably the reference value derived from a reference population as described hereinabove, is a TREM-1 level, in particular a sTREM-1 level, preferably a blood, plasma or serum level, of about 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 pg/mL. In one embodiment, the reference value, preferably the reference value derived from a reference population as described hereinabove, is a TREM-1 level, in particular a sTREM-1 level, preferably a blood, plasma or serum level, of about 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 pg/mL as determined using an ELISA, or a corresponding TREM-1 level, in particular a corresponding sTREM-1 level, preferably a blood, plasma or serum level, as determined using another immunoassay, in particular an ECLIA.

In one embodiment, the reference value, preferably the reference value derived from a reference population as described hereinabove, is a TREM-1 level, in particular a sTREM-1 level, preferably a blood, plasma or serum level, of about 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 885, 890, 895, 900, 905, 910, 915, 920, 925, 930, 935, 940, 945, 950, 955, 960, 965, 970, 975, 980, 985, 990, 995, or 1000 pg/mL as determined using an ECLIA, or a corresponding TREM-1 level, in particular a corresponding sTREM-1 level, preferably a blood, plasma or serum level, as determined using another immunoassay, in particular an ELISA.

In one embodiment, as described hereinabove, a severe form of the disease caused by a coronavirus, in particular a severe form COVID-19, is defined as requiring hospitalization and/or requiring admission in ICU. In one embodiment, a severe form of the disease caused by a coronavirus, in particular a severe form COVID-19, is defined as requiring respiratory support as described hereinabove. In one embodiment, a severe form of the disease caused by a coronavirus, in particular a severe form COVID-19, is defined as requiring invasive mechanical ventilation as described hereinabove.

In one embodiment, the present invention relates to an in vitro method for determining the severity of a disease caused by a coronavirus, in particular of COVID-19, in a subject, said method comprising or consisting of:
measuring the level of sTREM-1 in a biological sample from the subject as described hereinabove; and
comparing the level of sTREM-1 measured in the biological sample from the subject to a reference value, preferably a reference sTREM-1 level derived from a reference population of subjects who are substantially healthy as described hereinabove, or derived from a reference population of subjects diagnosed or identified as suffering, or having suffered, from a disease caused by a coronavirus, in particular from COVID-19 caused by SARS-CoV-2, as described hereinabove,
   wherein the higher the level of sTREM-1 measured in the biological sample from the subject is as compared to the reference value as described hereinabove, the more severe the disease caused by a coronavirus, in particular COVID-19, is in said subject.

According to one embodiment, the method of the invention comprises measuring the level of TREM-1, in particular of sTREM-1, in a biological sample from the subject as described hereinabove on at least two occasions, preferably separated by at least 24 hours. In other words, in one embodiment, the method of the invention comprises measuring the level of TREM-1, in particular of sTREM-1, in a biological sample from the subject as described hereinabove several times over time as described hereinabove. As mentioned hereinabove, it is to be understood that, in one embodiment, the method of the invention comprises measuring the level of TREM-1, in particular of sTREM-1, in several biological samples obtained from the subject as described hereinabove over time

Thus, in one embodiment, the method of the invention allows the monitoring over time of the disease caused by a coronavirus infection in the subject.

Another object of the invention is thus an in vitro method for monitoring a disease caused by a coronavirus as described hereinabove in a subject, said method comprising or consisting of:
measuring the level of TREM-1, in particular of sTREM-1, in a biological sample from the subject as described hereinabove on at least two occasions, preferably separated by at least 24 hours; and
comparing the level of TREM-1, in particular of sTREM-1, measured in the biological sample from the subject to a reference value.

In one embodiment, the present invention relates to an in vitro method for monitoring COVID-19 in a subject, said method comprising or consisting of:
measuring the level of sTREM-1 in a biological sample from the subject as described hereinabove on at least two occasions, preferably separated by at least 24 hours; and
comparing the level of sTREM-1 measured in the biological sample from the subject to a reference value.

In one embodiment, the method of the invention comprises measuring the level of TREM-1, in particular of sTREM-1, in a biological sample from the subject as described hereinabove on at least two occasions separated by at least 24 h, 36 h, 48 h, 60 h or 72 h. In one embodiment, the method of the invention comprises measuring the level of TREM-1, in particular of sTREM-1, in a biological sample from the subject as described hereinabove on at least two occasions separated by at least 1, 2, or 3 days.

As indicated above, it is to be understood that measuring the level of TREM-1, in particular of sTREM-1, in a biological sample from the subject as described hereinabove on at least two occasions means measuring the level of TREM-1, in particular of sTREM-1, in biological samples from the subject as described hereinabove obtained on at least two occasions.

In one embodiment, the method of the invention comprises measuring the level of TREM-1, in particular of sTREM-1, in a biological sample from the subject as described hereinabove every 24 h, every 36 h, every 48 h, every 60 h or every 72 h. In one embodiment, the method of the invention comprises measuring the level of TREM-1, in particular of sTREM-1, in a biological sample from the subject as described hereinabove every day, every 2 days or every 3 days.

According to one embodiment, a level of TREM-1, in particular of sTREM-1, measured in the biological sample from the subject which remains stable or increases over time is indicative of a worsening of the disease caused by a coronavirus as described hereinabove, in particular COVID-19, in the subject.

Accordingly, in one embodiment, a level of TREM-1, in particular of sTREM-1, measured in the biological sample from the subject which decreases over time is indicative of a lessening of the disease caused by a coronavirus as described hereinabove, in particular COVID-19, in the subject.

In one embodiment, the reference value is a personalized reference value, i.e., the reference value is a TREM-1 level, in particular a sTREM-1 level, measured in a biological sample obtained from the subject. In one embodiment, the personalized reference value is a TREM-1 level, in particular a sTREM-1 level, measured in a biological sample obtained from the subject at baseline, i.e., a baseline TREM-1 or sTREM-1 level. In one embodiment, the baseline TREM-1 or sTREM-1 level is a TREM-1 or sTREM-1 level measured in a biological sample obtained from the subject before the start of medical care. In one embodiment, the baseline TREM-1 or sTREM-1 level is a TREM-1 or sTREM-1 level measured in a biological sample obtained from the subject upon hospitalization or upon admission in ICU.

According to one embodiment, a level of TREM-1, in particular of sTREM-1, measured in the biological sample from the subject which is higher than the reference value as described hereinabove and which remains stable or increases over time is indicative of a worsening of the disease caused by a coronavirus as described hereinabove, in particular COVID-19, in the subject.

In one embodiment, the reference value is derived from the measure of the TREM-1 level, in particular of the sTREM-1 level, in a biological sample obtained from one or more subjects who are substantially healthy as defined hereinabove. In one embodiment, the reference value is a reference sTREM-1 level derived from a reference population of subjects who are substantially healthy.

In one embodiment, said reference value is a sTREM-1 level, preferably a blood, plasma or serum level, ranging from about 20 pg/mL to about 500 pg/mL, preferably from about 50 pg/mL to about 250 pg/mL, more preferably from about 100 pg/mL to about 200 pg/mL. In one embodiment, said reference value is a sTREM-1 level, preferably a blood, plasma or serum level, ranging from about 20 pg/mL to about 500 pg/mL, preferably from about 50 pg/mL to about 250 pg/mL, more preferably from about 100 pg/mL to about 200 pg/mL, as determined using an ELISA, or a corresponding TREM-1 level, in particular a corresponding sTREM-1 level, preferably a blood, plasma or serum level, as determined using another immunoassay, in particular an ECLIA. In one embodiment, said reference value is a sTREM-1 level, preferably a blood, plasma or serum level, of about 50, 75, 100, 125, 150, 175, 200, 225 or 250 pg/mL. In one embodiment, said reference value is a sTREM-1 level, preferably a blood, plasma or serum level, of about 50, 75, 100, 125, 150, 175, 200, 225 or 250 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value derived from a reference population of subjects who are substantially healthy is a TREM-1 level, in particular a sTREM-1 level, preferably a blood, plasma or serum level, ranging from about 50 pg/mL to about 500 pg/mL, preferably from about 200 pg/mL to about 450 pg/mL, more preferably from about 250 pg/mL to about 400 pg/mL, as determined using an ECLIA, or a corresponding TREM-1 level, in particular a corresponding sTREM-1 level, preferably a blood, plasma or serum level, as determined using another immunoassay, in particular an ELISA.

In one embodiment, the reference value derived from a reference population of subjects who are substantially healthy is a TREM-1 level, in particular a sTREM-1 level, preferably a blood, plasma or serum level, of about 50, 55, 60, 65, 70, 75, 80, 85, 90, 95, 100, 105, 110, 115, 120, 125, 130, 135, 140, 145, or 150 pg/mL. In one embodiment, the reference value derived from a reference population of subjects who are substantially healthy is a TREM-1 level, in particular a sTREM-1 level, preferably a blood, plasma or serum level, of about 50, 55, 60, 65, 70, 75, 80, 85, 90, 95, 100, 105, 110, 115, 120, 125, 130, 135, 140, 145, or 150 pg/mL as determined using an ELISA, or a corresponding TREM-1 level, in particular a corresponding sTREM-1 level, preferably a blood, plasma or serum level, as determined using another immunoassay, in particular an ECLIA. In one embodiment, the reference value derived from a reference population of subjects who are substantially healthy is a TREM-1 level, in particular a sTREM-1 level, preferably a blood, plasma or serum level, of about 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 pg/mL. In one embodiment, the reference value derived from a reference population of subjects who are substantially healthy is a TREM-1 level, in particular a sTREM-1 level, preferably a blood, plasma or serum level, of about 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 pg/mL as determined using an ELISA, or a corresponding TREM-1 level, in particular a corresponding sTREM-1 level, preferably a blood, plasma or serum level, as determined using another immunoassay, in particular an ECLIA.

In one embodiment, the reference value derived from a reference population of subjects who are substantially healthy is a TREM-1 level, in particular a sTREM-1 level, preferably a blood, plasma or serum level, of about 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355; 360, 365, 370, 375, 380, 385, 390, 395, 400, 405, 410, or 415 pg/mL as determined using an ECLIA, or a corresponding TREM-1 level, in particular a corresponding sTREM-1 level, preferably a blood, plasma or serum level, as determined using another immunoassay, in particular an ELISA.

In one embodiment, the reference value is derived from the measure of the TREM-1 level, in particular of sTREM-1 level in a biological sample from one or more subjects diagnosed or identified as suffering, or having suffered, from a disease caused by a coronavirus, in particular from COVID-19. In one embodiment, the reference value is a reference sTREM-1 level derived from a reference population of subjects diagnosed or identified as suffering, or having suffered, from a disease caused by a coronavirus, in particular from COVID-19.

In one embodiment, said reference value is a TREM-1 level, in particular a sTREM-1 level, preferably a blood, plasma or serum level, ranging from about 50 pg/mL to about 800 pg/mL, preferably from about 75 pg/mL to about 600 pg/mL, more preferably from about 100 pg/mL to about 400 pg/mL, even more preferably from about 130 pg/mL to about 400 pg/mL. In one embodiment, said reference value is a TREM-1 level, in particular a sTREM-1 level, preferably a blood, plasma or serum level, ranging from about 50 pg/mL to about 800 pg/mL, preferably from about 75 pg/mL to about 600 pg/mL, more preferably from about 100 pg/mL to about 400 pg/mL, even more preferably from about 130 pg/mL to about 400 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value, preferably the reference value derived from a reference population as described hereinabove, is a TREM-1 level, in particular a sTREM-1 level, preferably a blood, plasma or serum level, ranging from about 130 pg/mL to about 600 pg/mL, preferably from about 200 pg/mL to about 500 pg/mL, more preferably from about 250 pg/mL to about 400 pg/mL, even more preferably from about 300 pg/mL to about 375 pg/mL. In one embodiment, the reference value, preferably the reference value derived from a reference population as described hereinabove, is a TREM-1 level, in particular a sTREM-1 level, preferably a blood, plasma or serum level, ranging from about 130 pg/mL to about 600 pg/mL, preferably from about 200 pg/mL to about 500 pg/mL, more preferably from about 250 pg/mL to about 400 pg/mL, even more preferably from about 300 pg/mL to about 375 pg/mL, as determined using an ELISA, or a corresponding TREM-1 level, in particular a corresponding sTREM-1 level, preferably a blood, plasma or serum level, as determined using another immunoassay, in particular an ECLIA.

In one embodiment, the reference value, preferably the reference value derived from a reference population as described hereinabove, is a TREM-1 level, in particular a sTREM-1 level, preferably a blood, plasma or serum level, ranging from about 350 pg/mL to about 1200 pg/mL, preferably from about 600 pg/mL to about 1100 pg/mL, more preferably from about 700 pg/mL to about 1000 pg/mL, as determined using an ECLIA, or a corresponding TREM-1 level, in particular a corresponding sTREM-1 level, preferably a blood, plasma or serum level, as determined using another immunoassay, in particular an ELISA.

In one embodiment, said reference value is a TREM-1 level, in particular a sTREM-1 level, preferably a blood, plasma or serum level, of about 75, 100, 125, 150, 175, 200, 225, 250, 275, 300, 325, 350, 375 or 400 pg/mL. In one embodiment, said reference value is a TREM-1 level, in particular a sTREM-1 level, preferably a blood, plasma or serum level, of about 75, 100, 125, 150, 175, 200, 225, 250, 275, 300, 325, 350, 375 or 400 pg/mL as determined using an ELISA, or a corresponding TREM-1 level, in particular a corresponding sTREM-1 level, preferably a blood, plasma or serum level, as determined using another immunoassay, in particular an ECLIA. In one embodiment, the reference value, preferably the reference value derived from a reference population as described hereinabove, is a TREM-1 level, in particular a sTREM-1 level, preferably a blood, plasma or serum level, of about 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, or 430 pg/mL. In one embodiment, the reference value, preferably the reference value derived from a reference population as described hereinabove, is a TREM-1 level, in particular a sTREM-1 level, preferably a blood, plasma or serum level, of about 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, or 430 pg/mL as determined using an ELISA, or a corresponding TREM-1 level, in particular a corresponding sTREM-1 level, preferably a blood, plasma or serum level, as determined using another immunoassay, in particular an ECLIA. In one embodiment, the reference value, preferably the reference value derived from a reference population as described hereinabove, is a TREM-1 level, in particular a sTREM-1 level, preferably a blood, plasma or serum level, of about 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 pg/mL. In one embodiment, the reference value, preferably the reference value derived from a reference population as described hereinabove, is a TREM-1 level, in particular a sTREM-1 level, preferably a blood, plasma or serum level, of about 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 pg/mL as determined using an ELISA, or a corresponding TREM-1 level, in particular a corresponding sTREM-1 level, preferably a blood, plasma or serum level, as determined using another immunoassay, in particular an ECLIA.

In one embodiment, the reference value, preferably the reference value derived from a reference population as described hereinabove, is a TREM-1 level, in particular a sTREM-1 level, preferably a blood, plasma or serum level, of about 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 885, 890, 895, 900, 905, 910, 915, 920, 925, 930, 935, 940, 945, 950, 955, 960, 965, 970, 975, 980, 985, 990, 995, or 1000 pg/mL as determined using an ECLIA, or a corresponding TREM-1 level, in particular a corresponding sTREM-1 level, preferably a blood, plasma or serum level, as determined using another immunoassay, in particular an ELISA.

In one embodiment, the present invention relates to an in vitro method for monitoring a disease caused by a coronavirus, in particular COVID-19, in a subject, said method comprising or consisting of:
measuring the level of sTREM-1 in a biological sample from the subject as described hereinabove on at least two occasions, preferably separated by at least 24 hours; and
comparing the level of sTREM-1 measured in the biological sample from the subject to a reference value, preferably a personalized reference value such as a sTREM-1 level measured in a biological sample obtained from the subject at baseline,
   wherein a level of sTREM-1 measured in the biological sample from the subject which remains stable or increases over time is indicative of a worsening of the disease caused by a coronavirus as described hereinabove, in particular COVID-19, in the subject.

In one embodiment, the present invention relates to an in vitro method for monitoring a disease caused by a coronavirus, in particular COVID-19, in a subject, said method comprising or consisting of:
measuring the level of sTREM-1 in a biological sample from the subject as described hereinabove on at least two occasions, preferably separated by at least 24 hours; and
comparing the level of sTREM-1 measured in the biological sample from the subject to a reference value, preferably a reference sTREM-1 level derived from a reference population of subjects who are substantially healthy as described hereinabove, or derived from a reference population of subjects diagnosed or identified as suffering, or having suffered, from a disease caused by a coronavirus, in particular from COVID-19 caused by SARS-CoV-2, as described hereinabove,
   wherein a level of sTREM-1 measured in the biological sample from the subject which is higher than the reference value as described hereinabove and which remains stable or increases over time is indicative of a worsening of the disease caused by a coronavirus as described hereinabove, in particular COVID-19, in the subject.

The present invention also relates to a method for providing an adapted care to a subject suffering from a disease caused by a coronavirus, in particular COVID-19, identified as being at risk of having or developing a severe form and/or a complication of the disease caused by a coronavirus, in particular COVID-19, or at risk of death, said method comprising or consisting of:
identifying a subject suffering from a disease caused by a coronavirus, in particular COVID-19, as being at risk of having or developing a severe form and/or a complication of the disease caused by a coronavirus, in particular COVID-19, or at risk of death as described hereinabove; and
providing an adapted care to the subject suffering from a disease caused by a coronavirus, in particular COVID-19, identified as being at risk of having or developing a severe form and/or a complication of the disease caused by a coronavirus, in particular COVID-19, or at risk of death.

Another object of the invention is a method for providing an adapted care to a subject suffering from a disease caused by a coronavirus, in particular COVID-19, identified as having a poor prognosis, said method comprising or consisting of:
identifying a subject suffering from a disease caused by a coronavirus, in particular COVID-19, as having a poor prognosis as described hereinabove; and
providing an adapted care to the subject suffering from a disease caused by a coronavirus, in particular COVID-19, identified as having a poor prognosis.

Another object of the invention is a method for providing an adapted care to a subject suffering from a disease caused by a coronavirus, in particular COVID-19, identified as having a severe form of the disease caused by a coronavirus, in particular COVID-19, said method comprising or consisting of:
identifying a subject suffering from a disease caused by a coronavirus, in particular COVID-19, as having a severe form of the disease caused by a coronavirus, in particular COVID-19, as described hereinabove; and
providing an adapted care to the subject suffering from a disease caused by a coronavirus, in particular COVID-19, identified as having a severe form of the disease caused by a coronavirus, in particular COVID-19.

In one embodiment, providing an adapted care to the subject suffering from a disease caused by a coronavirus, in particular COVID-19, identified as described hereinabove comprises or consists of at least one of the following:
monitoring the subject, in particular monitoring the subject as described hereinabove;
providing respiratory support to the subject, in particular providing supplemental oxygen, non-invasive ventilation (NIV), or invasive mechanical ventilation (IMV) to the subject;
admitting the subject in intensive care unit (ICU).

In one embodiment, monitoring the subject comprises or consists of monitoring the respiratory function of the subject, for example through the monitoring of his/her oxygen saturation, of his/her arterial blood gases and/or venous blood gases, or his/her ratio of artery partial pressure of oxygen/inspired oxygen fraction or PaO2/FiO2 (mmHg).

In one embodiment, monitoring the subject comprises or consists of monitoring the organ function of the subject, for example through the determination of his/her SOFA score (Sequential Organ Failure Assessment (SOFA) score originally referred to as the Sepsis-related Organ Failure Assessment). The SOFA scoring system (Vincent et al., Crit Care Med. 1998 November; 26(11):1793-800) relies on the assessment of the respiratory system (i.e., PaO2/FiO2 (mmHg)); of the nervous system (i.e., Glasgow coma scale); of the cardiovascular system (i.e., mean arterial pressure or administration of vasopressors required); of the liver function (i.e., bilirubin (mg/dL or μmon)); of coagulation (i.e., platelet count); and of the kidney function (i.e., creatinine (mg/dL or μmon) or urine output (mL/d)).

The present invention also relates to a method for treating a subject suffering from a disease caused by a coronavirus, in particular COVID-19, identified as being at risk of having or developing a severe form and/or a complication of the disease caused by a coronavirus, in particular COVID-19, or at risk of death, said method comprising or consisting of:
identifying a subject suffering from a disease caused by a coronavirus, in particular COVID-19, as being at risk of having or developing a severe form and/or a complication of the disease caused by a coronavirus, in particular COVID-19, or at risk of death as described hereinabove; and
treating the subject suffering from a disease caused by a coronavirus, in particular COVID-19, identified as being at risk of having or developing a severe form and/or a complication of the disease caused by a coronavirus, in particular COVID-19, or at risk of death.

Another object of the invention is a method for treating a subject suffering from a disease caused by a coronavirus, in particular COVID-19, identified as having a poor prognosis, said method comprising or consisting of:
identifying a subject suffering from a disease caused by a coronavirus, in particular COVID-19, as having a poor prognosis as described hereinabove; and
treating the subject suffering from a disease caused by a coronavirus, in particular COVID-19, identified as having a poor prognosis.

Another object of the invention is a method for treating a subject suffering from a disease caused by a coronavirus, in particular COVID-19, identified as having a severe form of the disease caused by a coronavirus, in particular COVID-19, said method comprising or consisting of:
identifying a subject suffering from a disease caused by a coronavirus, in particular COVID-19, as having a severe form of the disease caused by a coronavirus, in particular COVID-19, as described hereinabove; and
treating the subject suffering from a disease caused by a coronavirus, in particular COVID-19, identified as having a severe form of the disease caused by a coronavirus, in particular COVID-19.

In one embodiment, treating the subject suffering from a disease caused by a coronavirus, in particular COVID-19, identified as described hereinabove comprises or consists of administering to said subject at least one of the following: respiratory support as defined hereinabove, vasopressor therapy (such as for example phenylephrine, norepinephrine, epinephrine, vasopressin, and/or dopamine), fluid therapy, antimicrobial therapy, antiviral therapy, cardiovascular support, renal replacement therapy, sedation, or any mixes thereof.

In one embodiment, treating the subject suffering from a disease caused by a coronavirus, in particular COVID-19, identified as described hereinabove comprises or consists of administering to said subject an antiviral agent, an anti-interleukin 6 (anti-IL-6) agent, steroids, another agent such as chloroquine or hydroxychloroquine, or any mixes thereof. In one embodiment, treating the subject suffering from a disease caused by a coronavirus, in particular COVID-19, identified as described hereinabove comprises or consists of administering to said subject an antiviral agent, an anti-interleukin 6 (anti-IL-6) agent, another agent such as chloroquine or hydroxychloroquine, or any mixes thereof.

In one embodiment, treating the subject suffering from a disease caused by a coronavirus, in particular COVID-19, identified as described hereinabove comprises or consists of administering to said subject at least one of the following: respiratory support as defined hereinabove, vasopressor therapy (such as for example phenylephrine, norepinephrine, epinephrine, vasopressin, and/or dopamine), fluid therapy, antimicrobial therapy, antiviral therapy, cardiovascular support, renal replacement therapy, sedation, an antiviral agent, an anti-interleukin 6 (anti-IL-6) agent, or another agent such as chloroquine or hydroxychloroquine, or any mixes thereof.

Example of antiviral agents include, without being limited to, remdesivir, and a combination of lopinavir and ritonavir (lopinavir/ritonavir). In one embodiment, treating the subject suffering from a disease caused by a coronavirus, in particular COVID-19, identified as described hereinabove comprises or consists of administering to said subject remdesivir, or a combination of lopinavir and ritonavir (lopinavir/ritonavir).

As used herein, anti-IL-6 agents target either IL-6 (interleukin 6 or interleukin-6) or its receptor (IL-6R). Example of anti-IL-6 agents include, without being limited to, tocilizumab and sarilumab. In one embodiment, treating the subject suffering from a disease caused by a coronavirus, in particular COVID-19, identified as described hereinabove comprises or consists of administering to said subject tocilizumab or sarilumab.

In one embodiment, the at least one further pharmaceutically active agent is a steroid, such as dexamethasone.

In one embodiment, treating the subject suffering from a disease caused by a coronavirus, in particular COVID-19, identified as described hereinabove comprises or consists of administering to said subject at least one of the following: remdesivir, a combination of lopinavir and ritonavir, tocilizumab, sarilumab, a steroid such as dexamethasone, chloroquine, hydroxychloroquine, or any mixes thereof. In one embodiment, treating the subject suffering from a disease caused by a coronavirus, in particular COVID-19, identified as described hereinabove comprises or consists of administering to said subject at least one of the following: remdesivir, a combination of lopinavir and ritonavir, tocilizumab, sarilumab, chloroquine, hydroxychloroquine, or any mixes thereof.

### BRIEF DESCRIPTION OF THE DRAWINGS

FIGS. 1A-1B are a combination of graphs comparing the levels of sTREM-1 in healthy volunteers (HV) and in patients suffering from COVID-19. FIG. 1A shows the levels of sTREM-1 at baseline (day 1). FIG. 1B shows the levels of sTREM-1 at day 3 following admission in intensive care unit (ICU). ***: p<0.001; ****: p<0.0001.

FIGS. 2A-2B are a combination of graphs comparing the levels of sTREM-1 in patients suffering from COVID-19 who survived (survivors—S) and in patients suffering from COVID-19 who did not survive (non-survivors—NS). FIG. 2A shows the levels of sTREM-1 at day 1 (i.e., day of admission in ICU). FIG. 2B shows the levels of sTREM-1 at day 3 following admission in intensive care unit (ICU). *: p<0.05; **: p<0.01.

FIGS. 3A-3B are a combination of graphs comparing the levels of sTREM-1 in healthy volunteers (HV) and in different groups of patients suffering from COVID-19: all: all patients suffering from COVID-19; MV<10: patients suffering from COVID-19 who were under mechanical ventilation for less than 10 days; MV≥10 and NS: patients suffering from COVID-19 who were under mechanical ventilation for 10 days or more, and patients who did not survive (non-survivors—NS) regardless of the duration of mechanical ventilation; MV<15: patients suffering from COVID-19 who were under mechanical ventilation for less than 15 days; MV≥15 and NS: patients suffering from COVID-19 who were under mechanical ventilation for 15 days or more, and patients who did not survive (non-survivors—NS) regardless of the duration of mechanical ventilation. FIG. 3A shows the levels of sTREM-1 at day 1 (i.e., day of admission in ICU). FIG. 3B shows the levels of sTREM-1 at day 3 following admission in ICU. ns: non-significant; *: p<0.05; ***: p<0.001; ****: p<0.0001.

FIGS. 4A-4B are a combination of graphs showing the levels of sTREM-1 in patients suffering from COVID-19 over time (from day 1 until day 21 following admission in ICU). FIG. 4A compares the levels of sTREM-1 over time in patients suffering from COVID-19 who were under mechanical ventilation for less than 15 days (MV<15) and in patients suffering from COVID-19 who were under mechanical ventilation for 15 days or more (MV≥15). FIG. 4B compares the levels of sTREM-1 over time in patients suffering from COVID-19 with troponin levels at baseline lower than 12 pg/mL (TROPO<12) and in patients suffering from COVID-19 with troponin levels at baseline higher than 12 pg/mL (TROPO>12). Missing data were treated with the LOCF method (Last Observed Carry Forward value).

FIG. 5A is a graph showing the ROC curve assessing the ability of sTREM-1 levels at day 3 following admission in ICU to discriminate between a mechanical ventilation (MV) lasting less than 15 days and a mechanical ventilation (MV) lasting 15 days or more. FIG. 5B is a table showing the parameters associated with the ROC curve.

FIGS. 6A-6D are a combination of incidence curves (Cox proportional-hazard (PH) model for individual classifiers and Kaplan-Meier for the overall cohort) showing the proportion over time of patients suffering from COVID-19 becoming free from invasive mechanical ventilation (i.e., begin extubated) or the proportion over time of patients suffering from COVID-19 exiting the intensive care unit. FIG. 6A is an incidence curve showing the proportion over time of patients suffering from COVID-19 becoming free from invasive mechanical ventilation (i.e., begin extubated) depending on a cut-off sTREM-1 level at day 1 (baseline) of 186 pg/mL. FIG. 6B is an incidence curve showing the proportion over time of patients suffering from COVID-19 becoming free from invasive mechanical ventilation (i.e., begin extubated) depending on a cut-off sTREM-1 level at day 3 of 186 pg/mL. FIG. 6C is an incidence curve showing the proportion over time of patients suffering from COVID-19 exiting the intensive care unit depending on a cut-off sTREM-1 level at day 1 (baseline) of 186 pg/mL. FIG. 6D is an incidence curve showing the proportion over time of patients suffering from COVID-19 exiting the intensive care unit depending on a cut-off sTREM-1 level at day 3 of 186 pg/mL. KM overall: proportion over time for the overall cohort of patients; sTREM-1>186: proportion over time for the patients with a sTREM-1 level at the indicated time (day 1 or day 3)>186 pg/mL; sTREM-1<186: proportion over time for the patients with a sTREM-1 level at the indicated time (day 1 or day 3)<186 pg/mL.

FIG. 7 is a flow chart depicting the validation cohort. Severe illness was defined as the need for ICU admission during hospital stay. Data are presented as median with interquartile range.

FIGS. 8A-C are a combination of graphs showing the plasma levels of sTREM-1 in different groups of patients with COVID-19. FIG. 8A compares the plasma sTREM-1 levels in moderately ill patients (moderate illness) and in severely ill patients (severe illness). Severe illness was defined as the need for ICU admission during hospital stay. FIG. 8B compares the plasma sTREM-1 levels in survivors and in non-survivors. FIG. 8C compares the plasma sTREM-1 levels in patients with a thromboembolic event (TEE) and in patients without a thromboembolic event (no TEE). Data are presented as median with interquartile ranges. P-values were calculated with Mann-Whitney U tests. *: p<0.05; ***: p<0.001.

FIGS. 9A-D are a combination of graphs showing the plasma levels of sTREM-1 in different groups of patients with COVID-19. FIG. 9A shows the plasma sTREM-1 levels in moderately ill patients with COVID-19 according to outcome (survivors vs. non-survivors, p-value=0.019). FIG. 9B compares the plasma sTREM-1 levels in severely ill patients with COVID-19 according to outcome (survivors vs. non-survivors, p-value=0.007). FIG. 9C compares the plasma sTREM-1 levels in surviving patients with COVID-19 according to illness severity (moderate illness vs. severe illness, p-value=0.068). FIG. 9D compares the plasma sTREM-1 levels in non-surviving patients with COVID-19 according to illness severity (moderate illness vs. severe illness, p-value=0.737). Severe illness was defined as the need for ICU admission during hospital stay. Data are presented as median with interquartile range. P-values were calculated with Mann-Whitney U tests. ns: non-significant; *: p<0.05; **: p<0.01.

FIGS. 10A-C are a combination of graphs showing the correlations between sTREM-1 levels and other clinical outcomes in patients with COVID-19. FIG. 10A shows the correlation between sTREM-1 levels and total length of hospital stay. FIG. 10B shows the correlation between sTREM-1 levels and total length of ICU stay. FIG. 10C shows the correlation between sTREM-1 levels and illness duration at time of sampling. Correlation coefficients and p-values, as indicated on the figures, were calculated using Spearman's rank correlation test.

FIGS. 11A-F are a combination of graphs showing correlations between sTREM-1 levels and inflammatory markers in patients with COVID-19. FIG. 11A shows the correlation between sTREM-1 levels and white blood cell (WBC) count. FIG. 11B shows the correlation between sTREM-1 levels and lymphocyte count. FIG. 11C shows the correlation between sTREM-1 levels and the levels of C-reactive protein (CRP). FIG. 11D shows the correlation between sTREM-1 levels and ferritin levels. FIG. 11E shows the correlation between sTREM-1 levels and D-dimer levels. FIG. 11F shows the correlation between sTREM-1 levels and interleukin-6 (IL-6) circulating concentration. Correlation coefficients and p-values, as indicated on the figures, were calculated using Spearman's rank correlation test.

FIGS. 12A-D are a combination of receiver-operating characteristic (ROC) curves assessing the ability of the indicated biomarkers (measured in the first sample obtained after COVID-19 diagnosis in the hospital) to discriminate between survivors and non-survivors. FIG. 12A is a ROC curve assessing the ability of sTREM-1 levels to discriminate between survivors and non-survivors. FIG. 12B is a ROC curve assessing the ability of C-reactive protein (CRP) levels to discriminate between survivors and non-survivors. FIG. 12C is a ROC curve assessing the ability of ferritin levels to discriminate between survivors and non-survivors. FIG. 12D is a ROC curve assessing the ability of interleukin-6 (IL-6) circulating concentration to discriminate between survivors and non-survivors. AUC: area under the curve.

FIGS. 13A-E are a combination of Kaplan-Meier curves showing the percentage survival over time of patients suffering from COVID-19 depending on the indicated biomarker cut-off values. FIG. 13A shows the percentage survival over time of patients suffering from COVID-19 depending on the indicated sTREM-1 cut-off value. FIG. 13B shows the percentage survival over time of patients suffering from COVID-19 depending on the indicated C-reactive protein (CRP) cut-off value. FIG. 13C shows the percentage survival over time of patients suffering from COVID-19 depending on the indicated ferritin cut-off value. FIG. 13D shows the percentage survival over time of patients suffering from COVID-19 depending on the indicated interleukin-6 (IL-6) cut-off value. FIG. 13E the percentage survival over time of patients suffering from COVID-19 depending on the indicated combinations of sTREM-1 and interleukin-6 (IL-6) cut-off values. Cut-off values were based on the maximal Youden's index derived from the receiver-operating characteristic curves shown on FIGS. 12A-D. Hazard ratios were calculated with the log-rank (Mantel-Cox) test.

FIG. 14 is a scatter plot of the sTREM-1 plasma concentrations measured with the sTREM-1 Elecsys ECLIA and with the sTREM-1 Quantikine ELISA.

### EXAMPLES

The present invention is further illustrated by the following examples.

### Example 1

### Materials and Methods

### Study Design and Participants

27 adult patients diagnosed with COVID-19 according to WHO (World Health Organization) interim guidance and admitted to intensive care unit (ICU) were included between Mar. 15th 2020 and Mar. 31St 2020 in the Centre Hospitalier Régional Universitaire (CHRU) of Nancy, France. All patients were confirmed as positive for SARS-CoV-2 by polymerase chain reaction (nasal/throat swab or pulmonary sample), and were admitted in ICU for acute respiratory distress syndrome. This study was approved by the CHRU Nancy ethic committee (saisine n° 196).

### Definitions

Secondary infection was diagnosed in patients showing clinical signs or symptoms of pneumonia or bacteremia confirmed by positive culture. Acute respiratory distress syndrome (ARDS) was diagnosed according to the Berlin Definition (ARDS Definition Task Force, Ranieri et al., JAMA. 2012; 307(23):2526-2533.). Acute kidney injury was diagnosed according to the kidney disease improving global outcomes (KDIGO) clinical practice guidelines (Khwaja, Nephron Clin Pract. 2012; 120(4):c179-c184). Cardiocirculatory failure was diagnosed if serum levels of troponin were above 12 pg/mL, or if there was a need for vasopressors.

### Data Collection and Laboratory Procedures

Medical records were collected and retrospectively analyzed. Plasma samples were collected from ICU admission to discharge at day 1, 3, 6, 14, and 21. Blood count, hematology, serum biochemical tests, troponins, and interleukin-6 (IL-6) were obtained by routine blood tests. Inflammatory markers were quantified in plasma by enzyme-linked immunosorbent assay (ELISA) and multiplex assay.

sTREM-1 Measurement

sTREM-1 levels were measured using an analytically validated ELISA-based method (EMA Guideline on bioanalytical method validation. EMEA/CHMP/EWP/192217/2009 (2011)) using a commercially available research use only ELISA assay (Human TREM-1 Quantikine® ELISA kit, R&D Systems, reference DTRM10C). The analytical performances and acceptance criteria of this method are summarized in Table 1 below.

TABLE 1
Summary of the analytical performances and acceptance
criteria of the sTREM-1 ELISA validated method

| Analyte | sTREM-1 | |
|---|---|---|
| Matrix | Human K₂-EDTA plasma | |
| Analytical method | ELISA | |
| Calibration standards | 15.6#, 31.3 (LCS), 62.5, 125, 250, 500, 750, 1000, | |
| (two wells per CS including | 2000 (HCS) and 3000# pg/mL | |
| anchoring points#) in assay | The imprecision (% CV), must be ≤20% for CS | |
| diluent from the kit | ranged from 31.3 (LCS) to 2000 pg/mL (HCS). | |
| | The inaccuracy (% RE) must be within ±20% | |
| | (±25% at the LCS and the HCS). | |
| | No more than two CS (30%) may be excluded from | |
| | the calibration curve, which must contain finally at | |
| | least 6 calibration concentration levels, including the | |
| | LCS and HCS. | |
| LLOQ in human K25-EDTA | 31.3 pg/mL | |
| plasma | | |
| ULOQ in human K2-EDTA | 1800 pg/mL | |
| plasma | | |
| QC samples | QC.Low (101 pg/mL), QC.Mid (532 pg/mL) and | |
| (n = 2 duplicates, unspiked | QC.High (1080 pg/mL) | |
| or spiked samples prepared | % CV must be ≤20% and % RE must be within ±20%. | |
| in undiluted or diluted | At least 4/6 QC samples must be within acceptance | |
| human K₂-EDTA plasma) | criteria (2/6 QC samples, not both at the same | |
| | concentration, may be outside the acceptance | |
| | criteria). | |
| Minimum required dilution | None | |
| (MRD) | | |
| Dilution linearity | Up to 1/100 in calibrator diluent RD5-18 | |
| Specificity against | No interference was demonstrated in any of the | |
| endogenous matrix | blank human K₂-EDTA plasma samples tested. | |
| components | | |
| Stability in human K₂-EDTA | Freeze/thaw (F/T) | 3 cycles at −24° C. ± 6° C. |
| plasma | stability | 3 cycles at −75° C. ± 10° C. |
| | Short-term stability | 24 hours at room |
| | | temperature |
| | | 24 hours at +5° C. ± 5° C. |
| | Long-term (LT) stability | 670 days at −24° C. ± 6° C. |
| | | 670 days at −75° C. ± 10° C. |

LCS: lowest calibration standard; HCS: highest calibration standard; CS: calibration standard(s); QC: quality control; RE: relative error; CV: coefficient of variation; LLOQ: lower limit of quantification; ULOQ: upper limit of quantification.

### Cytokines Assays

The Cytometric Bead Array (CBA) Human Soluble Protein Flex Set System (BD Biosciences, San Jose, Calif., USA) was used for the simultaneous detection of 12 cytokines/chemokines and endothelial markers (IL-1β (interleukin-1(3), IL-6 (interleukin-6), IL-8 (interleukin-8), IL-10 (interleukin-10), MCP-1 (monocyte chemoattractant protein-1), INFγ (interferon γ), RANTES (regulated on activation, normal T cell expressed and secreted), VEGF (vascular endothelium growth factor), ICAM-1 (intercellular adhesion molecule 1), E-selectin, P-selectin and VCAM-1 (vascular cell adhesion molecule 1)) in plasma. The CBA technique was performed according to the manufacturer's instructions. The detection sensitivities were 36.06 pg/mL for ICAM-1, E-selectin, P-selectin, VCAM-1 and 9.77 pg/mL for IL-1β, IL-6, IL-8, IL-10, MCP-1, INFγ, RANTES, VEGF. Data were analyzed using the FCAP Array Infinite software (SoftFlow Group, Hungary). Alternatively, plasma concentrations of Ang1 (angiopoietin-1) and Ang2 (angiopoietin-2) were measured by commercially available specific enzyme-linked immunosorbent assay (Human Angiopoietin-1/Angiopoietin-2 Quantikine® ELISA Kit, R&D Systems), according to the manufacturer's instructions. The detection limits were 10.3 pg/mL (Ang 1) and 21.3 pg/mL (Ang2).

### Statistical Analysis

Categorical variables were expressed as number (%) and compared by χ² test between groups. Continuous variables were expressed as median (interquartile range) and compared with the Mann-Whitney U test. Receiver-operating characteristic (ROC) curves were constructed to illustrate cut-off values of sTREM-1.

Time-to-event analyses were performed by Kaplan-Meier survival analysis, in which a penalty for death was applied. Missing values were replaced by using the last observation carried forward (LOCF) method when indicated.
Time to ICU exit (exit from intensive care unit) and time to becoming IVM free (extubated from invasive mechanical ventilation, i.e., time to extubation) analyses were conducted separately. Subjects censored due to death were considered as being censored at day 35 for the time-to-event analyses. The Product-Limit Estimates (Kaplan-Meier curves) were estimated first, without any consideration of sTREM-1 class. Subsequently Cox proportional hazard (PH) models were fitted separately for each of these categorical predictors: sTREM-1 classifier at day 1 (baseline), and sTREM-1 classifier at day 3. From each of the Cox PH models, the hazards ratios for the endpoints were produced along with their 95% confidence intervals and p-values from a log-rank test. From each Cox PH model, the cumulative incidence function at each classifier level was plotted and overlaid alongside the Kaplan-Meier curve.
ROC (Receiver Operator Characteristics) curves were used to investigate the sTREM-1 as a classification factor (i) for MV (Mechanical Ventilation) duration of <15 days or (ii) for MV duration of >15 days and non-survivors, as a supportive analysis. The sensitivity (true positive rate) and specificity (true negative rate) were explored using these curves.
The sTREM-1 values at day 1 and day 3 were used in this analysis, separately. The Area Under the Curve (AUC) of the ROC curve was used to assess performance of sTREM-1.

### Results

### Healthy Volunteers Characteristics

Characteristics of the healthy volunteers are shown in Table 2 below.

TABLE 2
Characteristics of the healthy volunteers

| | Healthy volunteers (n = 21) | |
|---|---|---|
| Age (years) | 42 | (22-48.5) |
| Gender (n, %) | | |
| Male | 9 | (43) |
| Female | 12 | (57) |
| BMI (kg/m²) | NA | |
| Comorbidity (n, %) | | |
| Diabetes mellitus | NA | |
| Cardiovascular disease | | |
| Pulmonary disease | | |
| Malignancy | | |
| Length of hospital stay (days) | NA | |
| MV duration (days) | NA | |
| Mortality (n, %) | NA | |
| sTREM-1 (pg/mL) | 104 | (75-124) |

| | |
|---|---|
| IL-6 (pg/mL) | NA |

Data are presented as median (IQR) or n (%). BMI: body mass index. MV: mechanical ventilation. IL-6: interleukin-6.

### Patients Characteristics

Baseline characteristics are shown in Table 3 below.

TABLE 3
Baseline characteristics of the patients included in the cohort

| | | | | | MV ≥ 15 | | |
|---|---|---|---|---|---|---|---|
| | All patients | | MV < 15 days | | days and NS | | p- |
| | (n = 27) | | (n = 11) | | (n = 16) | | value |
| Characteristics | | | | | | | |
| Age (years) | 63 | (56-71) | 62 | (52-71) | 64 | (59-71) | 0.267 |
| BMI (kg/m²) | 28.9 | (26.1-31.18) | 29.65 | (25.95-31.18) | 27.95 | (26.1-32.55) | 0.5845 |
| Male, n (%) | 19 | (70.37%) | 5 | (45.45%) | 14 | (87.50%) | 0.0187 |
| Comorbidities | | | | | | | |
| Hypertension, n (%) | 11 | (40.74%) | 3 | (27.27%) | 8 | (50.00%) | 0.2376 |
| Diabetes, n (%) | 4 | (14.81%) | 0 | (0.00%) | 4 | (25.00%) | 0.0724 |
| CV disease, n (%) | 1 | (3.70%) | 0 | (0.00%) | 1 | (6.25%) | 0.3981 |
| Pulmonary disease, n (%) | 1 | (3.70%) | 0 | (0.00%) | 1 | (6.25%) | 0.3981 |
| Cardiocirculatory failure | | | | | | | |
| Vasopressors, n (%) | 22 | (81.48%) | 9 | (81.82%) | 13 | (81.25%) | 0.9993 |
| TROPO > 12 pg/mL, n (%) | 17 | (62.96%) | 6 | (54.55%) | 11 | (68.75%) | 0.9702 |
| AKI | | | | | | | |
| KDIGO 1 (Creat 15-30), n (%) | 0 | (0.00%) | 0 | (0.00%) | 0 | (0.00%) | 0.223 |
| KDIGO 2 (Creat 30-40), n (%) | 0 | (0.00%) | 0 | (0.00%) | 0 | (0.00%) | |
| KDIGO 3 (Creat >40 or RRT), n (%) | 2 | (7.41%) | 0 | (0.00%) | 2 | (12.50%) | |
| ARDS | | | | | | | |
| PaO₂/FiO₂ ratio <100, n (%) | 10 | (37.04%) | 2 | (18.18%) | 8 | (50.00%) | 0.0457 |
| PaO₂/FiO₂ ratio 100-200, n (%) | 13 | (48.15%) | 6 | (54.55%) | 8 | (50.00%) | |
| PaO₂/FiO₂ ratio 200-300, n (%) | 4 | (14.81%) | 3 | (27.27%) | 0 | (0.00%) | |
| Outcome | | | | | | | |
| Secondary Infection, n (%) | 9 | (33.33%) | 1 | (9.09%) | 8 | (50.00%) | 0.0267 |
| VAP, n (%) | 8 | (29.63%) | 0 | (0.00%) | 8 | (50.00%) | |
| Length of stay (days) | 17 | (9-19) | 10 | (8-12) | 18 | (17-21.75) | 0.001 |
| MV duration (days) | 15 | (8-17) | | | | | |

Unless indicated, values are presented as median (interquartile range). P-values are comparisons between mechanical ventilation (MV) < 15 days subgroup and MV ≥ 15 days and non-survivors (NS) subgroup. BMI: body mass index. VAP: ventilator-associated pneumonia. MV: mechanical ventilation. NS: non-survivors. CV disease: cardiovascular disease. Creat: creatine. RRT: renal replacement therapy. ARDS: acute respiratory distress syndrome. TROPO: troponin levels (pg/mL). AKI: acute kidney injury. KDIGO: kidney disease improving global outcomes. PaO₂/FiO₂ ratio: ratio of artery partial pressure of oxygen/inspired oxygen fraction.

Among the 27 patients of this cohort, 22 patients required vasopressors and 17 had cardiac troponin levels above 12 pg/mL, 2 patients had a KDIGO of 3, 10 patients presented with severe ARDS, 13 with moderate ARDS, and 4 with mild ARDS. 9 patients developed secondary infection, of whom 8 were suffering from ventilator-associated pneumonia (VAP). 3 patients died, and the median duration of mechanical ventilation (MV) was 15 days. This value was unchanged when a penalty of 33 days was attributed for MV duration for non-survivors, which corresponds to the maximum duration of MV observed in that cohort.

11 patients had MV duration below 15 days, and 16 had at least 15 days of MV or died. A higher proportion of male and a higher proportion of patients with severe ARDS had MV duration of at least 15 days or died. These patients had a higher frequency of secondary infections.
No significant differences in baseline and day 3 hematology and biochemistry parameters were observed between these two groups, with an exception for white blood cells count (WBC) and neutrophils at day 3, but values were within normal range.
sTREM-1 Levels were More Elevated in COVID-19 Patients than in Healthy Volunteers (HV), Both at Baseline and at Day 3

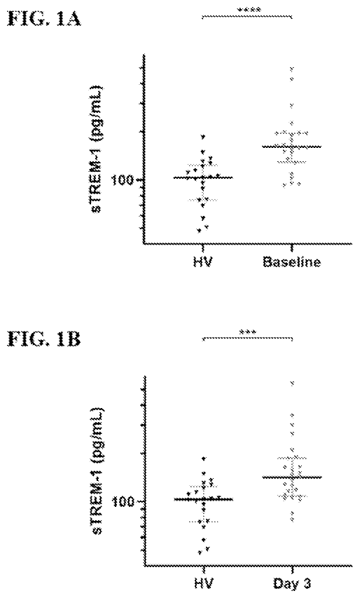

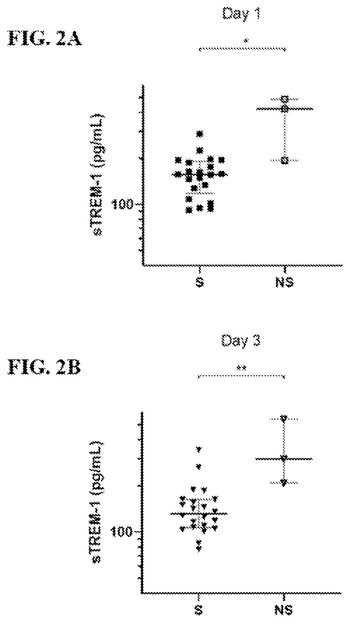

The median sTREM-1 plasma concentration in healthy volunteers (HV) was 103.5 pg/mL (IQR (interquartile range) 75.22-124.4), and median sTREM-1 plasma concentration in COVID-19 patients was 161.1 pg/mL (IQR 129.4-195.7) at baseline, and 142 pg/mL (IQR 108.7-187.3) at day 3 (see FIG. 1). As shown on FIGS. 1A-B, the difference between the sTREM-1 plasma concentration of HV and that of COVID-19 patients was statistically different. Interestingly, even if only 3 patients died, significantly higher sTREM-1 levels were observed in non-survivors (see FIG. 2) as compared to sTREM-1 levels in COVID-19 patients who survived, both at baseline (see FIG. 2A) and at day 3 (see FIG. 2B). The median sTREM-1 plasma concentration in non-survivors (NS) was 422.6 pg/mL at baseline, and 300 pg/mL at day 3.

Higher sTREM-1 Levels were Observed at Baseline (Day 1) and Day 3 in Patients Who Required Longer Duration Under Mechanical Ventilation (MV).

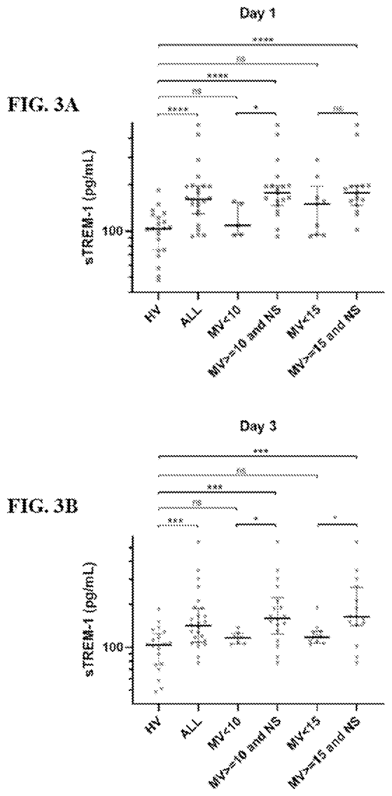

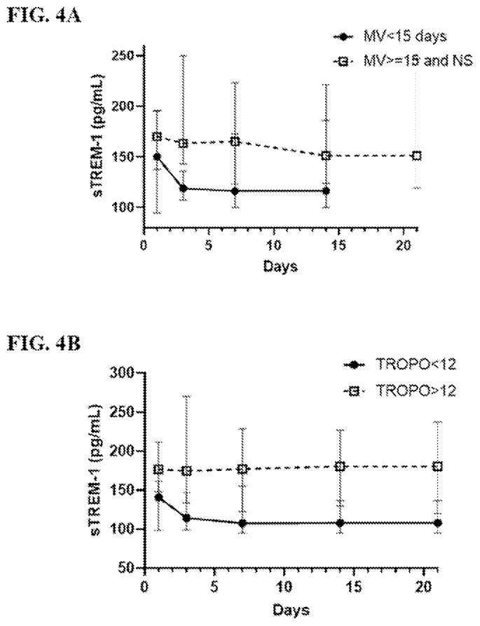

Indeed, at baseline and day 3, a significant difference in sTREM-1 plasma concentration was observed in the subgroup including patients who did not survive and patients who required 10 days or more under MV, as compared to the subgroup of patients who required less than 10 days under MV. This difference was still present in the subgroup including non-survivors and patients who required 15 days or more under MV, as compared to the subgroup of patients who required less than 15 days under MV (see FIGS. 3A-B and Table 4 below). This was further confirmed when looking at the kinetics of sTREM-1 in these two subgroups (see FIG. 4A). Indeed, non-survivors and patients with 15 days or more under MV showed more elevated levels of sTREM-1 from baseline (day 1) to discharge (day 21), as compared to patients who required less than 15 days under MV.

TABLE 4
Hematology data, biochemistry data, and inflammatory markers in all COVID-19 patients, and in the two
subgroups defined by either less than 15 days under MV, or by death or at least 15 days under MV

| | | | |
|---|---|---|---|
| All patients | MV < 15 days | MV ≥ 15 days and NS | p- |
| (n = 27) | (n = 11) | (n = 16) | value |

Hematology

| | | | | | | | | |
|---|---|---|---|---|---|---|---|---|
| WBC (G/L) | D 1 | 7380 | (6400-9640) | 6830 | (6310-7630) | 8405 | (6518-10375) | 0.1244 |
| | D 3 | 7690 | (6493-9393) | 7310 | (5020-8500) | 8390 | (6940-11370) | 0.0428 |
| Monocytes (G/L) | D 1 | 300 | (170-410) | 340 | (240-440) | 215 | (170-402.5) | 0.3108 |
| | D 3 | 435 | (277.5-827.5) | 420 | (270-880) | 450 | (280-810) | 0.8284 |
| Ly (G/L) | D 1 | 6403 | (440-1010) | 640 | (400-1010) | 655 | (492.5-1050) | 0.8179 |
| | D 3 | 870 | (485-1368) | 1010 | (530-1620) | 860 | (350-1200) | 0.4127 |
| PMN (G/L) | D 1 | 6420 | (4950-8080) | 5570 | (4840-6820) | 7310 | (4985-8215) | 0.1988 |
| | D 3 | 5830 | (5005-7225) | 4990 | (4320-5830) | 6450 | (5700-8620) | 0.0009 |
| PLT (G/L) | D 1 | 230500 | (165500-257250) | 240500 | (206750-257250) | 197000 | (146750-261500) | 0.1654 |
| | D 3 | 252500 | (185750-344500) | 290000 | (205000-335000) | 251000 | (163000-352000) | 0.413 |

Biochemistry

| | | | | | | | | |
|---|---|---|---|---|---|---|---|---|
| Hg (g/dL) | D 1 | 12.4 | (10.9-13.8) | 12.4 | (10.7-13.4) | 12.75 | (10.95-13.88) | 0.4731 |
| | D 3 | 11.3 | (10.38-12.05) | 10.7 | (10-11.6) | 11.8 | (10.5-12.8) | 0.0666 |
| Bilirubin (mg/L) | D 1 | 7 | (5-8) | 5 | (5-7) | 8 | (6-8.75) | 0.0931 |
| | D 3 | 6 | (4-12) | 4.5 | (4-8.5) | 7 | (5-13) | 0.2867 |
| AST (U/L) | D 1 | 71 | (47-106) | 74 | (24-116) | 70 | (52.75-100.3) | 0.8557 |
| | D 3 | 56.5 | (43.25-117.5) | 52 | (39-159) | 64 | (47-103) | 0.5232 |
| Creatinine kinase (U/L) | D 1 | 198.5 | (74.25-535.3) | 105 | (46.5-341) | 275 | (106-836) | 0.2093 |
| | D 3 | 138 | (33-399) | 61 | (34-289) | 149.5 | (31.5-598.8) | 0.9048 |
| TP (g/L) | D 1 | 56 | (51-59) | 56 | (51-59) | 56 | (51.25-59) | 0.7793 |
| | D 3 | 54.5 | (51.25-57) | 54 | (51.5-55) | 56 | (51-58) | 0.3393 |
| Lactate (mmol/L) | D 1 | 1.2 | (0.6-1.8) | 1 | (0.7-1.7) | 1.2 | (0.9-1.575) | 0.7611 |
| | D 3 | 1.1 | (0.9-1.6) | 1.35 | (0.8-1.6) | 1.1 | (1-1.7) | 0.88 |

Inflammatory markers (pg/mL)

| | | | | | | | | |
|---|---|---|---|---|---|---|---|---|
| sTREM-1 | D 1 | 161.1 | (129.4-195.7) | 150 | (94.48-194.9) | 176.8 | (147-195.9) | 0.1383 |
| | D 3 | 142 | (108.7-187.3) | 117.4 | (106.8-129.9) | 163.4 | (142-263.7) | 0.0375 |
| ICAM-1 | D 1 | 79141 | (64361-118402) | 79337 | (67209-152074) | 78945 | (64004-120051) | 0.7702 |
| | D 3 | 109646 | (78067-149311) | 110651 | (66034-185491) | 109646 | (84864-148100) | 0.935 |
| E-Selectin | D 1 | 8047 | (5467-12088) | 6945 | (4133-10925) | 8330 | (6698-12224) | 0.2384 |
| | D 3 | 7676 | (4590-10856) | 6284 | (4308-9382) | 7676 | (6257-11019) | 0.4865 |
| P-Selectin | D 1 | 7387 | (5297-20841) | 8965 | (5366-23250) | 7342 | (4840-20436) | 0.482 |
| | D 3 | 9227 | (7206-16571) | 10649 | (7117-21825) | 8019 | (7079-15062) | 0.683 |
| VCAM-1 | D 1 | 404614 | (356629-470779) | 393329 | (356685-409567) | 446181 | (313891-478054) | 0.446 |
| | D 3 | 375892 | (293199-455877) | 377341 | (308508-453248) | 366593 | (241842-475257) | 0.683 |
| IL-6 | D 1 | 172.2 | (94.57-231.7) | 145.7 | (97.2-342.4) | 184.4 | (61.2-221.2) | 0.9534 |
| | D 3 | 194.6 | (112.4-372) | 126.2 | (95.47-335.5) | 216.7 | (119.4-395.8) | 0.3669 |
| IL-8 | D 1 | 49.37 | (39.04-64.63) | 49.28 | (33.77-72.63) | 53.24 | (44.55-64.96) | 0.6824 |
| | D 3 | 43.91 | (25.48-63.12) | 34.62 | (22.97-54.91) | 46.86 | (25.61-83.87) | 0.311 |
| IL-10 | D 1 | 5.225 | (1.583-18.38) | 5.1 | (2.28-21.03) | 5.35 | (0-19.97) | 0.6077 |
| | D 3 | 0 | (0-8.52) | 4.265 | (0-9.708) | 0 | (0-7.53) | 0.4605 |
| MCP-1 | D 1 | 294.5 | (189.4-515.3) | 197.9 | (108-363.8) | 400.7 | (266.8-571.6) | 0.0732 |
| | D 3 | 194 | (81.23-506.1) | 168.2 | (48.07-258.2) | 282.3 | (165.2-566) | 0.091 |
| RANTES | D 1 | 10023 | (8309-15045) | 11737 | (8308-17880) | 9840 | (8303-11594) | 0.446 |
| | D 3 | 11923 | (10013-15182) | 12307 | (10872-15660) | 11437 | (9730-14929) | 0.4284 |
| Ang1 | D 1 | 10768 | (7530-13344) | 11508 | (7653-14140) | 9243 | (7328-13588) | 0.5985 |
| | D 3 | 11880 | (7988-14845) | 11451 | (8042-14203) | 12053 | (6878-16380) | 0.8918 |
| Ang2 | D 1 | 2103 | (1672-2862) | 1711 | (1591-2853) | 2132 | (1768-2870) | 0.446 |
| | D 3 | 4517 | (3014-6115) | 3780 | (2743-5046) | 4803 | (3655-7090) | 0.238 |
| Ang2/Ang1 | D 1 | 0.18 | (0.14-0.3425) | 0.15 | (0.125-0.3) | 0.19 | (0.14-0.49) | 0.2688 |
| | D 3 | 0.39 | (0.255-0.535) | 0.325 | (0.185-1.8475 | 0.4 | (0.27-0.46) | 0.4695 |

sTREM-1

| | | | | | | | |
|---|---|---|---|---|---|---|---|
| D 3 − D 1 | −2.995 | (−36.47-18.83) | −9.675 | (−35.06-15.86) | 1.655 | (−38.85-42.96) | 0.9203 |
| % D 3 − D 1 | −2.785 | (−20.02-13.33) | −7.09 | (−19.01-17.1) | 0.87 | (−20.43-15.13) | 0.9734 |

P-values are comparisons between the MV < 15 days subgroup and the MV ≥ 15 days and non-survivors (NS) subgroup. MV: mechanical ventilation. NS: non-survivors. D 1: day 1. D 3: day 3. WBC: white blood cells. Ly: lymphocytes. PMN: polymorphonuclear neutrophils. PLT: platelets. Hg: hemagglutinin. AST: aspartate aminotransferase TP: total proteins. ICAM-1: intercellular adhesion molecule 1. VCAM-1: vascular cell adhesion molecule 1. IL-6: interleukin 6. IL-8: interleukin-8. IL-10: interleukin-10. MCP-1: monocyte chemoattractant protein-1. RANTES: regulated on activation, normal T cell expressed and secreted. Ang1: angiopoietin-1. Ang2: angiopoietin-2. D3-D1 is the difference between sTREM-1 concentration at day 3 and day 1. % D3-D1 is the percentage of that difference compared to the value at D1.

The median duration of mechanical ventilation (MV) was 15 days in the study cohort (see Table 3 above). As indicated above, 11 patients had MV duration below 15 days, and 16 had at least 15 days of MV or died. As shown on FIGS. 3A-B, patients who required less than 15 days under MV had similar sTREM-1 levels at baseline and day 3 (150 pg/mL (IQR: 94,48-194.9) and 117.4 pg/mL (IQR:106.8-129.9), respectively) as compared to healthy volunteers (103.5 pg/mL (IQR:75.22-124.4)). Strikingly, patients requiring 15 days or more under MV and non-survivors had significantly higher sTREM-1 levels at baseline (176.8 pg/mL (IQR:147-195.9), p<0.0001) and at day 3 (163.4 pg/mL (IQR:142-263.7), p=0.0003) as compared to healthy volunteers (see FIGS. 3A-B), and had significantly higher sTREM-1 levels at day 3 as compared to patients who required less than 15 days incidence under MV (see FIG. 3B).

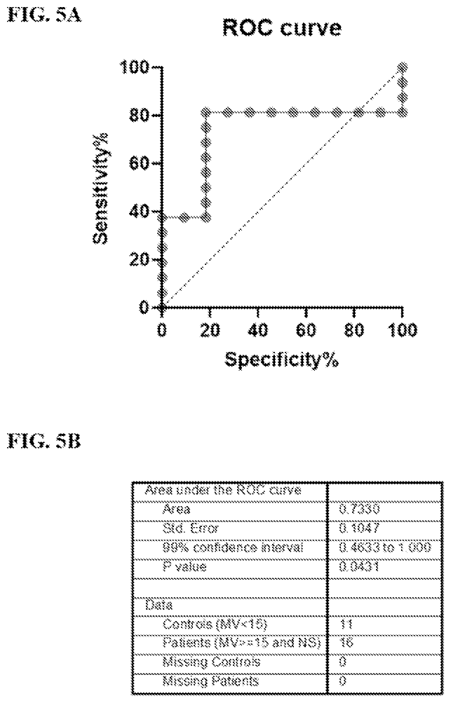

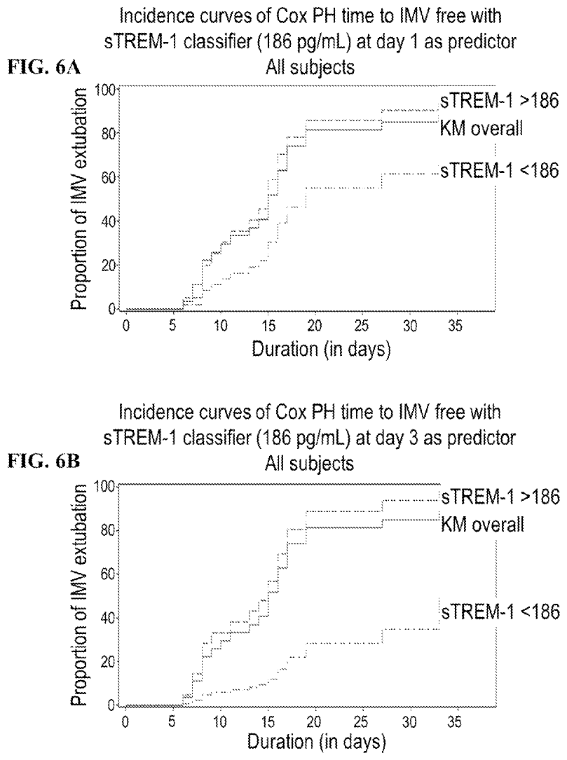

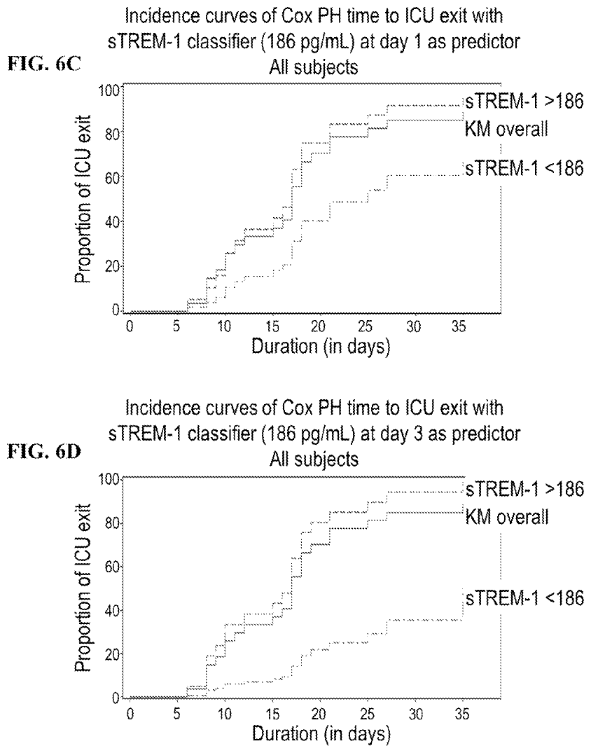

As shown in FIGS. 5A-B, sTREM-1 concentration at day 3 yielded a discriminative value in identifying patients with MV needs below 15 days from patients with MV needs above 15 days, with an area under the curve of 0.733 (95% confidence interval 0.4633-1.000) and a p-value of 0.0431. A sTREM-1 cut-off value of 186 pg/mL provided approximately 80% specificity and 90% sensitivity. Based on the sTREM-1 cut-off value of 186 pg/mL, analyses of the time to ICU exit and time to IMV free (i.e., time to extubation from IMV) were performed. For time to becoming IVM free (i.e., extubated from invasive mechanical ventilation), the hazard ratio (HR) and its respective 95% CI was 0.411 (0.155,1.087) at baseline (day 1) (see FIG. 6A), and 0.153 (0.034,0.685) at day 3 (see FIG. 6B). For time to ICU exit (i.e., exit from intensive care unit), the HR and its respective 95% CI was 0.374 (0.141,0.992) at baseline (day 1) (see FIG. 6C), and 0.151 (0.033, 0.681) at day 3 (see FIG. 6D).

The data thus show that COVID-19 patients displaying sTREM-1 levels at baseline and day 3 above 186 pg/mL had lower likelihood to be extubated before 15 days or to survive (baseline HR: 0.374 (95% CI 0.141-0.992), day3 HR: 0.151 (95% CI 0.033-0.681)).

Correlations Between sTREM-1 and Other Clinical Parameters

To further determine whether TREM-1 pathway activation was associated with complications in COVID-19, correlations between sTREM-1 and clinical parameters and features of COVID-19 were assessed at day 1 and day 3 (see Tables 5-7 below).

Lymphocyte levels have been found to be predictor of prognosis in COVID-19 patients, and individuals who died of COVID-19 are demonstrated to have had expressively lower lymphocyte levels than survivors. Interestingly, at day 3, sTREM-1 showed a significant inverse correlation with lymphocyte counts in all COVID-19 patients (spearman r −0.6593 (−0.8530 to −0.3058), p-value=0.0012, see Table 5). This inverse correlation was lost when looking at less severely ill patients requiring less than 15 days under MV (see Table 6), but was still present in more severely ill patients (spearman r −0.5699 (−0.8425 to −0.06476), p-value 0.0291, see Table 7). sTREM-1 levels were also found to be correlated with bilirubin levels at day 3 (spearman r 0.5196 (0.07123 to 0.7933) p-value 0.0226, see Table 5), as well as with urea and creatine levels (see below Table 5, Table 6, and Table 7), different markers of liver and renal suffering or dysfunction. A positive correlation was also found between sTREM-1 and troponin levels in all patients (r=0.6559 p=0.0096, see Table 5), and this correlation was even more pronounced in the more severely ill patients (r=−0.8333, p=0.0154, see Table 7). This association between TREM-1 pathway activation and cardiac failure was confirmed by looking at the kinetics of sTREM-1 in patients presenting with troponins levels above 12 pg/mL: they showed higher levels of sTREM-1 from inclusion (day 1) to discharge (day 21), as compared to patients presenting with troponin levels below 12 pg/mL (see FIG. 4B).

Organ dysfunction, and in particular renal, liver and/or cardiac dysfunction, is a major complication in COVID-19 patients, associated with complicated and poor outcome. The data confirm that TREM-1 pathway engagement may drive inflammation and complications in COVID-19 patients.

No detectable levels of INF-γ, IL-1β, and VEGF, elevated levels of ICAM-1, E-selectin, P-selectin, VCAM-1, IL-6, IL-8, IL-10, MCP-1, RANTES, Ang1, and Ang2 were observed in the patients of the cohort (see Table 4 above). Unexpectedly, sTREM-1 was the only marker that showed a difference between the two groups of patients defined by either less than 15 days under MV, or by death or at least 15 days under MV. The difference was more pronounced, and statistically significant, at day 3.

TABLE 5
Correlation table between sTREM-1 and clinical and
inflammatory markers in all COVID-19 patients

| Correlation table | | All patients (n = 27) | | | | |
|---|---|---|---|---|---|---|
| sTREM-1 with | | Spearman r (95% IC) | | p-value | n | |
| WBC | D 1 | 0.3662 | (−0.05629 to 0.6774) | 0.0784 | 24 | |
| | D 3 | −0.1364 | (−0.5461 to 0.3261) | 0.5556 | 21 | |
| Monocytes | D 1 | −0.04224 | (−0.4483 to 0.3783) | 0.8446 | 24 | |
| | D 3 | −0.2762 | (−0.6406 to 0.1898) | 0.2256 | 21 | |
| Lymphocytes | D 1 | −0.3484 | (−0.6663 to 0.07656) | 0.0952 | 24 | |
| | D 3 | −0.6593 | (−0.8530 to −0.3058) | 0.0012 | 21 | ** |
| Neutrophils | D 1 | 0.3487 | (−0.07624 to 0.6664) | 0.0949 | 24 | |
| | D 3 | 0.1494 | (−0.3142 to 0.5554) | 0.5182 | 21 | |
| Platelets | D 1 | −0.1391 | (−0.5229 to 0.2916) | 0.5167 | 24 | |
| | D 3 | −0.4208 | (−0.7279 to 0.02698) | 0.0575 | 21 | |
| Hemoglobin | D 1 | 0.01174 | (−0.4041 to 0.4236) | 0.9566 | 24 | |
| | D 3 | 0.1222 | (−0.3389 to 0.5359) | 0.5978 | 21 | |
| ALAT | D 1 | −0.08612 | (−0.4828 to 0.3399) | 0.6891 | 24 | |
| | D 3 | 0.009785 | (−0.4459 to 0.4615) | 0.9673 | 20 | |
| ASAT | D 1 | −0.09702 | (−0.4912 to 0.3302) | 0.652 | 24 | |
| | D 3 | 0.006769 | (−0.4484 to 0.4591) | 0.9774 | 20 | |
| Bilirubin | D 1 | 0.3545 | (−0.06964 to 0.6701) | 0.0892 | 24 | |
| | D 3 | 0.5196 | (0.07123 to 0.7933) | 0.0226 | 19 | * |
| Urea | D 1 | 0.677 | (0.3655 to 0.8521) | 0.0003 | 24 | *** |
| | D 3 | 0.5751 | (0.2212 to 0.7952) | 0.0026 | 25 | ** |
| Creatine | D 1 | 0.7306 | (0.4540 to 0.8788) | <0.0001 | 24 | **** |
| | D 3 | 0.7372 | (0.4732 to 0.8797) | <0.0001 | 25 | **** |
| Total Protein | D 1 | 0.2311 | (−0.2022 to 0.5887) | 0.2773 | 24 | |
| | D 3 | 0.003493 | (−0.4110 to 0.4168) | 0.9871 | 24 | |
| Lactate | D 1 | 0.002182 | (−0.4121 to 0.4157) | 0.9919 | 24 | |
| | D 3 | 0.1833 | (−0.2401 to 0.5480) | 0.3806 | 25 | |
| Troponin | D 1 | 0.3926 | (−0.02547 to 0.6938) | 0.0577 | 24 | |
| | D 3 | 0.6559 | (0.2003 to 0.8783) | 0.0096 | 15 | ** |
| ICAM-1 | D 1 | −0.193 | (−0.5621 to 0.2401) | 0.3661 | 24 | |
| | D 3 | −0.01615 | (−0.4189 to 0.3919) | 0.9389 | 25 | |
| E-selectin | D 1 | −0.213 | (−0.5762 to 0.2203) | 0.3175 | 24 | |
| | D 3 | 0.3701 | (−0.04169 to 0.6744) | 0.0686 | 25 | |
| P-selectin | D 1 | −0.1652 | (−0.5421 to 0.2670) | 0.4404 | 24 | |
| | D 3 | −0.1377 | (−0.5145 to 0.2836) | 0.5116 | 25 | |
| VCAM-1 | D 1 | −0.02783 | (−0.4367 to 0.3906) | 0.8973 | 24 | |
| | D 3 | 0.2169 | (−0.2068 to 0.5721) | 0.2976 | 25 | |
| IL-6 | D 1 | −0.02261 | (−0.4325 to 0.3950) | 0.9165 | 24 | |
| | D 3 | 0.3462 | (−0.06904 to 0.6591) | 0.0901 | 25 | |
| IL-8 | D 1 | 0.413 | (−0.001068 to 0.7062) | 0.0448 | 24 | * |
| | D 3 | 0.5123 | (0.1348 to 0.7599) | 0.0088 | 25 | ** |
| IL-10 | D 1 | −0.03967 | (−0.4463 to 0.3805) | 0.854 | 24 | |
| | D 3 | 0.3841 | (−0.02541 to 0.6832) | 0.058 | 25 | |
| MCP-1 | D 1 | 0.3991 | (−0.01773 to 0.6978) | 0.0533 | 24 | |
| | D 3 | 0.6508 | (0.3332 to 0.8357) | 0.0004 | 25 | *** |
| RANTES | D 1 | −0.2461 | (−0.5990 to 0.1869) | 0.2464 | 24 | |
| | D 3 | −0.3631 | (−0.6699 to 0.04975) | 0.0744 | 25 | |
| Ang1 | D 1 | 0.1148 | (−0.3141 to 0.5047) | 0.5933 | 24 | |
| | D 3 | −0.2354 | (−0.5850 to 0.1881) | 0.2574 | 25 | |
| Ang2 | D 1 | 0.1722 | (−0.2603 to 0.5471) | 0.4211 | 24 | |
| | D 3 | 0.2592 | (−0.1635 to 0.6015) | 0.2108 | 25 | |
| Ang2/Ang1 | D 1 | 0.09057 | (−0.3360 to 0.4863) | 0.6738 | 24 | |
| | D 3 | 0.3882 | (−0.02051 to 0.6858) | 0.0551 | 25 | |

WBC: white blood cells. ALAT: alanine aminotransferase. ASAT: aspartate aminotransferase. ICAM-1: intercellular adhesion molecule 1. VCAM-1: vascular cell adhesion molecule 1. IL-6: interleukin 6. IL-8: interleukin-8. IL-10: interleukin-10. MCP-1: monocyte chemoattractant protein-1. RANTES: regulated on activation, normal T cell expressed and secreted. Ang1: angiopoietin-1. Ang2: angiopoietin-2.
* indicates statistical significance with * p < 0.05, ** p < 0.01, *** p < 0.001, and **** p < 0.0005.

TABLE 6
Correlation table between sTREM-1 and clinical and inflammatory
markers in COVID-19 patients with MV duration below 15 days

| Correlation table | | MV < 15 (n = 11) | | | |
|---|---|---|---|---|---|
| sTREM-1 with | | Spearman r (95% IC) | p-value | n | |
| WBC | D 1 | 0.5667 | 0.1206 | 9 | |
| | D 3 | 0.1581 | 0.6621 | 10 | |
| Monocytes | D 1 | −0.08333 | 0.8432 | 9 | |
| | D 3 | 0.1337 | 0.7132 | 10 | |
| Lymphocytes | D 1 | −0.2167 | 0.5809 | 9 | |
| | D 3 | −0.1636 | 0.6567 | 10 | |
| Neutrophils | D 1 | 0.5667 | 0.1206 | 9 | |
| | D 3 | 0.3333 | 0.3487 | 10 | |
| Platelets | D 1 | −0.2167 | 0.5809 | 9 | |
| | D 3 | 0.5152 | 0.1334 | 10 | |
| Hemoglobin | D 1 | −0.2833 | 0.463 | 9 | |
| | D 3 | 0.1394 | 0.7072 | 10 | |
| ALAT | D 1 | 0.06667 | 0.8801 | 9 | |
| | D 3 | 0.3929 | 0.3956 | 7 | |
| ASAT | D 1 | 0.08368 | 0.833 | 9 | |
| | D 3 | 0.1071 | 0.8397 | 7 | |
| Bilirubin | D 1 | 0.1368 | 0.7311 | 9 | |
| | D 3 | 0.1518 | 0.8333 | 6 | |
| Urea | D 1 | 0.4603 | 0.2149 | 9 | |
| | D 3 | 0.3587 | 0.3067 | 10 | |
| Creatine | D 1 | 0.7667 | 0.0214 | 9 | * |
| | D 3 | 0.4303 | 0.2182 | 10 | |
| Total Protein | D 1 | −0.1681 | 0.6666 | 9 | |
| | D 3 | 0.1519 | 0.6974 | 9 | |
| Lactate | D 1 | 0.05858 | 0.886 | 9 | |
| | D 3 | −0.09847 | 0.7893 | 10 | |
| Troponin | D 1 | 0.35 | 0.3586 | 9 | |
| | D 3 | 0.1441 | 0.7603 | 7 | |
| ICAM-1 | D 1 | 0.2167 | 0.5809 | 9 | |
| | D 3 | 0.1152 | 0.7589 | 10 | |
| E-selectin | D 1 | −0.2333 | 0.5517 | 9 | |
| | D 3 | −0.6727 | 0.039 | 10 | * |
| P-selectin | D 1 | 0.2167 | 0.5809 | 9 | |
| | D 3 | −0.1152 | 0.7589 | 10 | |
| VCAM-1 | D 1 | −0.45 | 0.2298 | 9 | |
| | D 3 | 0.4545 | 0.1912 | 10 | |
| IL-6 | D 1 | 0.2667 | 0.4933 | 9 | |
| | D 3 | 0.1515 | 0.6821 | 10 | |
| IL-8 | D 1 | 0.55 | 0.1328 | 9 | |
| | D 3 | 0.2439 | 0.4936 | 10 | |
| IL-10 | D 1 | −0.03347 | 0.9377 | 9 | |
| | D 3 | 0.1741 | 0.8 | 10 | |
| MCP-1 | D 1 | 0.7167 | 0.0369 | 9 | * |
| | D 3 | 0.2364 | 0.5135 | 10 | |
| RANTES | D 1 | −0.06667 | 0.8801 | 9 | |
| | D 3 | −0.7333 | 0.0202 | 10 | * |
| Ang1 | D 1 | 0.2 | 0.6134 | 9 | |
| | D 3 | 0.3455 | 0.3304 | 10 | |
| Ang2 | D 1 | 0.3667 | 0.3363 | 9 | |
| | D 3 | 0.09091 | 0.8113 | 10 | |
| Ang2/Ang1 | D 1 | 0.2845 | 0.454 | 9 | |
| | D 3 | −0.103 | 0.785 | 10 | |

WBC: white blood cells. ALAT: alanine aminotransferase. ASAT: aspartate aminotransferase. ICAM-1: intercellular adhesion molecule 1. VCAM-1: vascular cell adhesion molecule 1. IL-6: interleukin 6. IL-8: interleukin-8. IL-10: interleukin-10. MCP-1: monocyte chemoattractant protein-1. RANTES: regulated on activation, normal T cell expressed and secreted. Ang1: angiopoietin-1. Ang2: angiopoietin-2.
* p < 0.05.

TABLE 7
Correlation table between sTREM-1 and clinical and inflammatory
markers in most severe COVID-19 patients, non-survivors
and patients with MV duration above 15 days

| Correlation table | | MV >= 15 and NS (n = 16) | | | | |
|---|---|---|---|---|---|---|
| sTREM-1 with | | Spearman r (95% IC) | | p-value | n | |
| WBC | D 1 | 0.175 | (−0.3848 to 0.6407) | 0.532 | 15 | |
| | D 3 | −0.2214 | (−0.6683 to 0.3429) | 0.4266 | 15 | |
| Monocytes | D 1 | −0.03943 | (−0.5525 to 0.4953) | 0.8926 | 15 | |
| | D 3 | −0.3596 | (−0.7438 to 0.2033) | 0.1873 | 15 | |
| Lymphocytes | D 1 | −0.3714 | (−0.7498 to 0.1901) | 0.1735 | 15 | |
| | D 3 | −0.5699 | (−0.8425 to −0.06476) | 0.0291 | 15 | * |
| Neutrophils | D 1 | 0.1321 | (−0.4216 to 0.6141) | 0.6389 | 15 | |
| | D 3 | −0.03571 | (−0.5499 to 0.4981) | 0.9031 | 15 | |
| Platelets | D 1 | −0.007143 | (−0.5297 to 0.5193) | 0.9847 | 15 | |
| | D 3 | −0.7286 | (−0.9066 to −0.3303) | 0.0029 | 15 | ** |
| Hemoglobin | D 1 | 0.08929 | (−0.4566 to 0.5863) | 0.7532 | 15 | |
| | D 3 | −0.05357 | (−0.5623 to 0.4845) | 0.8525 | 15 | |
| ALAT | D 1 | −0.1357 | (−0.6163 to 0.4186) | 0.6297 | 15 | |
| | D 3 | 0.4897 | (−0.1021 to 0.8255) | 0.0914 | 13 | |
| ASAT | D 1 | −0.2218 | (−0.6685 to 0.3425) | 0.4238 | 15 | |
| | D 3 | −0.07428 | (−0.6123 to 0.5107) | 0.8101 | 13 | |
| Bilirubin | D 1 | 0.2853 | (−0.2813 to 0.7044) | 0.3008 | 15 | |
| | D 3 | 0.233 | (−0.3806 to 0.7042) | 0.4404 | 13 | |
| Urea | D 1 | 0.6512 | (0.1924 to 0.8764) | 0.0101 | 15 | * |
| | D 3 | 0.6409 | (0.1754 to 0.8722) | 0.0118 | 15 | * |
| Creatine | D 1 | 0.5165 | (−0.01092 to 0.8191) | 0.0508 | 15 | |
| | D 3 | 0.7721 | (0.4162 to 0.9229) | 0.0011 | 15 | ** |
| Total Protein | D 1 | 0.4269 | (−0.1257 to 0.7773) | 0.1133 | 15 | |
| | D 3 | −0.2471 | (−0.6831 to 0.3187) | 0.3718 | 15 | |
| Lactate | D 1 | −0.2009 | (−0.6562 to 0.3617) | 0.4696 | 15 | |
| | D 3 | 0.2757 | (−0.2909 to 0.6991) | 0.3174 | 15 | |
| Troponin | D 1 | 0.2395 | (−0.3259 to 0.6787) | 0.3868 | 15 | |
| | D 3 | 0.8333 | ( ) | 0.0154 | 8 | * |
| ICAM-1 | D 1 | −0.2607 | (−0.6908 to 0.3056) | 0.3469 | 15 | |
| | D 3 | 0.3843 | (−0.1756 to 0.7563) | 0.1573 | 15 | |
| E-selectin | D 1 | −0.3607 | (−0.7444 to 0.2020) | 0.187 | 15 | |
| | D 3 | 0.01071 | (−0.5167 to 0.5322) | 0.9744 | 15 | |
| P-selectin | D 1 | −0.1679 | (−0.6363 to 0.3911) | 0.5492 | 15 | |
| | D 3 | 0.35 | (−0.2137 to 0.7389) | 0.2012 | 15 | |
| VCAM-1 | D 1 | 0.1464 | (−0.4095 to 0.6231) | 0.6024 | 15 | |
| | D 3 | 0.1643 | (−0.3942 to 0.6341) | 0.558 | 15 | |
| IL-6 | D 1 | −0.1643 | (−0.6341 to 0.3942) | 0.558 | 15 | |
| | D 3 | 0.625 | (0.1495 to 0.8657) | 0.0148 | 15 | * |
| IL-8 | D 1 | 0.3643 | (−0.1980 to 0.7462) | 0.1824 | 15 | |
| | D 3 | 0.6245 | (0.1487 to 0.8655) | 0.0152 | 15 | * |
| IL-10 | D 1 | −0.01261 | (−0.5336 to 0.5153) | 0.9664 | 15 | |
| | D 3 | −0.1268 | (−0.6107 to 0.4260) | 0.6571 | 15 | |
| MCP-1 | D 1 | 0.1036 | (−0.4451 to 0.5957) | 0.7144 | 15 | |
| | D 3 | 0.6357 | (0.1669 to 0.8701) | 0.0128 | 15 | * |
| RANTES | D 1 | −0.3071 | (−0.7163 to 0.2591) | 0.265 | 15 | |
| | D 3 | −0.2 | (−0.6557 to 0.3625) | 0.4738 | 15 | |
| Ang1 | D 1 | 0.09643 | (−0.4509 to 0.5910) | 0.7337 | 15 | |
| | D 3 | −0.4321 | (−0.7799 to 0.1194) | 0.1094 | 15 | |
| Ang2 | D 1 | −0.01071 | (−0.5322 to 0.5167) | 0.9744 | 15 | |
| | D 3 | 0.3357 | (−0.2291 to 0.7314) | 0.2212 | 15 | |
| Ang2/Ang1 | D 1 | −0.03757 | (−0.5512 to 0.4967) | 0.8952 | 15 | |
| | D 3 | 0.6845 | (0.2497 to 0.8896) | 0.0061 | 15 | ** |

WBC: white blood cells. ALAT: alanine aminotransferase. ASAT: aspartate aminotransferase. ICAM-1: intercellular adhesion molecule 1. VCAM-1: vascular cell adhesion molecule 1. IL-6: interleukin 6. IL-8: interleukin-8. IL-10: interleukin-10. MCP-1: monocyte chemoattractant protein-1. RANTES: regulated on activation, normal T cell expressed and secreted. Ang1: angiopoietin-1. Ang2: angiopoietin-2.
* indicates statistical significance with * p < 0.05, and ** p < 0.01.

### Example 2

### Materials and Methods

### Study Design and Participants

The retrospective study presented below was conducted on a validation cohort of 192 patients with COVID-19 (both ICU and non-ICU) admitted to the Radboud University Medical Centre (Radboudumc), Nijmegen, the Netherlands. The validation cohort consists of patients with a PCR-proven or clinically diagnosed SARS-CoV-2 infection admitted to the Radboudumc between 6 Mar. 6th 2020 and Apr. 15^{th} 2020. Clinical diagnosis of COVID-19 infection was defined based on signs and symptoms, specific computed tomography (CT) findings according to the Dutch COVID-19 Reporting and Data System (CO-RADS) classification, and final consensus of clinical experts. 95% (183/192) of the COVID-19-diagnosed patients had a positive PCR result at the time of diagnosis. The study protocol was approved by the local ethics committee (CMO 2020 6344 and CMO 2016 2963). All patients or legal representatives were informed about the study details and could decline to participate.

Ethylenediaminetetraacetic acid (EDTA) plasma was collected at the first routine blood withdrawal for laboratory testing after COVID-19 diagnosis in the hospital (i.e., baseline). For analysis, patients were stratified into groups based on disease severity and mortality. Disease severity was defined based on the need for ICU admission during hospital stay: severe illness was defined as patients needing ICU admission, and moderate illness was defined as patients without the need for ICU admission during hospital stay. patients were admitted to a non-ICU ward at the time of sampling, but needed ICU admission later during their hospital stay. These patients were classified in the severe illness group.

### Data Collection

Clinical data and laboratory results were collected from the electronic patient files (EPIC, EPIC Systems Corporation, Verona, Wis., USA) and recorded in electronic Case Report Forms (Castor EDC, Amsterdam, the Netherlands). Values of white blood cell (WBC) counts, lymphocyte counts, C-reactive protein (CRP), ferritin and D-dimer were collected at the day of plasma sampling. Clinical outcomes (ICU admission, hospital length of stay, ICU length of stay, incidence of thromboembolic events (TEE), and mortality) were recorded until hospital discharge.

### Measurements and Assays

Venous blood was collected in EDTA tubes and thereafter centrifuged at 2954 g (3800 rpm) at room temperature for 10 minutes. Plasma was collected and aliquoted before storage at −80° C. for further analysis. Concentrations of IL-6 were measured using enzyme-linked immunosorbent assays (ELISA, Quantikine, R&D systems) according to the manufacturer's instructions, with a lower detection limit of 16 pg/mL. Plasma sTREM-1 levels were measured using an analytically validated ELISA assay according to regulatory requirements (EMA 2011) using a commercially available research use only ELISA assay (Human TREM-1 Quantikine® ELISA kit, R&D Systems, reference DTRM10C). This method was validated with lower and upper limit of quantification of 34.2 and 2070 pg/mL, respectively. For routine analysis, each analytical run contained 3 levels of quality control (QC) sample (Low, Mid and High, with QC.Low=93 pg/mL, QC.Mid=728 pg/mL, and QC.High=1610 pg/mL), and each run was accepted if standard curve and QC samples are within acceptance criteria. The analytical performances and acceptance criteria of this method are summarized in Table 1 above (see Example 1).

### Statistical Analysis

The obtained data were analyzed using SPSS version 25.0 (IBM Corp., Armonk, N.Y., USA), GraphPad Prism version 8.0 (GraphPad Software, Inc., San Diego, Calif., USA), and MedCalc version 19.6.4 (MedCalc Software Ltd, Ostend, Belgium). Differences between groups were assessed by Mann-Whitney U tests for continuous variables and by Fisher's exact tests for discrete variables. Correlations between sTREM-1 concentration and inflammatory parameters, and sTREM-1 concentration and duration of hospital stay were assessed by Spearman's rank correlation tests. Receiver operating characteristic (ROC) analyses were performed to assess the prognostic performance of several biomarkers by calculating the area under the curve (AUC). The optimal cut-off values for the biomarkers were defined based on the maximal Youden's J index and used to assess differences in survival during hospital stay for high versus low biomarker concentrations by Kaplan-Meier survival analysis. Hazard ratios were based on the log-rank test. A p-value <0.05 (two-tailed) was considered statistically significant.

### Results

Elevated sTREM-1 Concentrations in Patients with Severe COVID-19

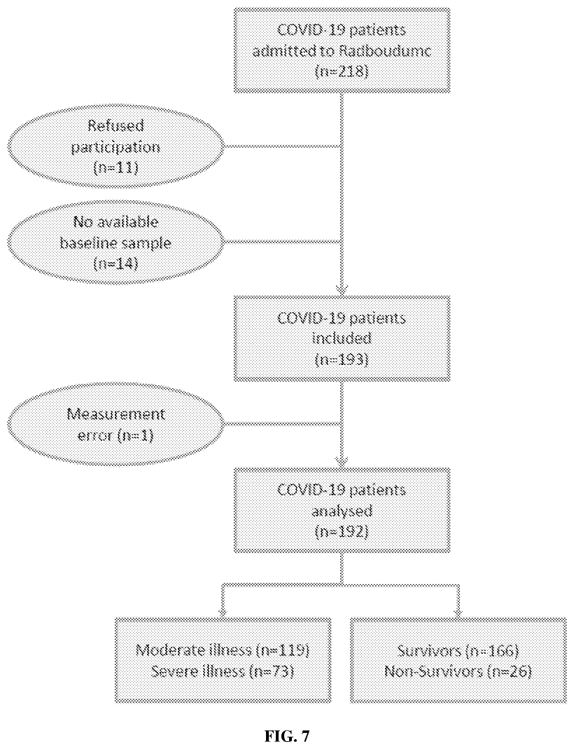

218 patients diagnosed with COVID-19 and admitted to the Radboudumc were assessed for study inclusion (FIG. 7). Of these, 11 patients refused to participate. Moreover, 14 other patients were excluded because no plasma sample was available from first routine blood withdrawal after COVID-19 diagnosis (baseline), and one patient was excluded due to a measurement error. The final study population (n=192) was divided in groups based on disease severity (moderate illness (n=119) and severe illness (n=73)) and outcome (survivors (n=166) and non-survivors (n=26)). 72 patients out of the 73 patients admitted in ICU (i.e., severely ill patients) required invasive mechanical ventilation. 109 patients out of the 119 patients who were moderately ill (i.e., no ICU admission) received oxygen therapy: 8 patients received non-invasive ventilation and 101 patients received supplemental oxygen by nasal cannula or mask. Table 8 below shows the characteristics of the COVID-19 patients, divided in groups according to disease severity (i.e., moderate illness/severe illness). Table 9 below shows the characteristics of COVID-19 patients, divided in groups of according to outcome (i.e., survivors/non-survivors).

TABLE 8
Characteristics of the patients included in the validation cohort (all
patients and patients divided in groups according to disease severity)

| | All patients | | Moderate illness | | Severe illness | | P- |
|---|---|---|---|---|---|---|---|
| | (n = 192) | | (n = 119) | | (n = 73) | | value^{a} |
| Age (years) | 65 | (54-72) | 66 | (53-73) | 64 | (57-71) | 0.285 |
| Gender (n, %) | | | | | | | |
| Male | 133 | (69) | 79 | (66) | 54 | (74) | 0.334 |
| Female | 59 | (31) | 40 | (34) | 19 | (26) | |
| BMI (kg/m²) | 26.4 | (24.0-29.0) | 26.0 | (23.7-28.9) | 26.8 | (24.7-29.3) | 0.250 |
| Comorbidity (n, %) | | | | | | | |
| Diabetes mellitus | 38 | (20) | 22 | (19) | 16 | (22) | 0.580 |
| Cardiovascular disease | 102 | (53) | 64 | (54) | 38 | (52) | 0.882 |
| Pulmonary disease | 39 | (20) | 31 | (26) | 8 | (11) | 0.016 |
| Malignancy | 48 | (25) | 34 | (29) | 14 | (19) | 0.171 |
| Days of illness at day of sampling (days) | 11 | (8-15) | 10 | (8-14) | 13 | (9-16) | 0.022 |
| ICU admission (n, %)^{b} | 73 | (38) | NA | | NA | | NA |
| Length of hospital stay (days) | 9 | (6-24) | 7 | (5-9) | 31 | (19-45) | <0.001 |
| Mortality (n, %) | 26 | (14) | 10 | (8) | 16 | (22) | 0.010 |
| sTREM-1 (pg/mL) | 208 | (151-292) | 195 | (139-283) | 235 | (176-319) | 0.017 |
| CRP (mg/L) | 109 | (60-173) | 81 | (41-120) | 175 | (128-291) | <0.001 |
| Ferritin (μg/L) | 1058 | (543-1879) | 822 | (399-1461) | 1694 | (935-2554) | <0.001 |
| IL-6 (pg/mL)^{c} | 72 | (28-118) | 43 | (23-82) | 144 | (79-405) | <0.001 |

Data are presented as median (IQR) or n (%). BMI: body mass index. CRP: C-reactive protein. IL-6: interleukin-6.
^{a}Moderate illness versus severe illness (Mann-Whitney U test),
^{b}ICU during total hospital admission,
^{c}Measured in 151 of the 192 patients.

TABLE 9
Characteristics of the patients included in the validation cohort
(all patients and patients divided in groups according to outcome)

| | All patients | | Survivors | | Non-survivors | | P- |
|---|---|---|---|---|---|---|---|
| | (n = 192) | | (n = 166) | | (n = 26) | | value^{a} |
| Age (years) | 65 | (54-72) | 64 | (53-71) | 73 | (69-75) | <0.001 |
| Gender (n, %) | | | | | | | |
| Male | 133 | (69) | 115 | (69) | 18 | (69) | 1.000 |
| Female | 59 | (31) | 51 | (31) | 8 | (31) | |
| BMI (kg/m²) | 26.4 | (24.0-29.0) | 26.6 | (23.9-29.3) | 25.8 | (24.0-28.4) | 0.651 |
| Comorbidity (n, %) | | | | | | | |
| Diabetes mellitus | 38 | (20) | 33 | (20) | 5 | (19) | 1.000 |
| Cardiovascular disease | 102 | (53) | 84 | (51) | 18 | (69) | 0.092 |
| Pulmonary disease | 39 | (20) | 31 | (19) | 8 | (31) | 0.188 |
| Malignancy | 48 | (25) | 41 | (25) | 7 | (27) | 0.810 |
| Days of illness at day of sampling (days) | 11 | (8-15) | 11 | (8-15) | 11 | (8-17) | 0.882 |
| ICU admission (n, %)^{b} | 73 | (38) | 57 | (34) | 16 | (62) | 0.010 |
| Length of hospital stay (days) | 9 | (6-24) | 9 | (6-24) | 15 | (6-15) | 0.339 |
| Mortality (n, %) | 26 | (14) | NA | | NA | | NA |
| sTREM-1 (pg/mL) | 208 | (151-292) | 199 | (142-278) | 326 | (207-445) | <0.001 |
| CRP (mg/L) | 109 | (60-173) | 103 | (56-172) | 139 | (83-210) | 0.154 |
| Ferritin (μg/L) | 1058 | (543-1879) | 996 | (491-1864) | 1270 | (717-1962) | 0.237 |
| IL-6 (pg/mL)^{c} | 72 | (28-118) | 67 | (26-104) | 188 | (70-480) | <0.001 |

Data are presented as median (IQR) or n (%). BMI: body mass index. CRP: C-reactive protein. IL-6: interleukin-6.
^{a}Survivors versus non-survivors (Mann Whitney U test),
^{b}ICU during total hospital admission,
^{c}Measured in 151 of the 192 patients.

As compared to moderately ill patients (i.e., patients only admitted to the clinical ward), severely ill patients had higher concentrations of inflammatory parameters (CRP, D-dimer and IL-6), a longer hospital stay (31 days versus 7 days, p<0.001), and a higher mortality rate (22% versus 8%, p=0.010). As compared to the patients who survived, non-survivors were older (73 years vs. 64 years, p<0.001) and were more frequently admitted to the ICU (62% vs. 34%, p=0.010). No other differences in gender, BMI, comorbidities were observed between the groups.

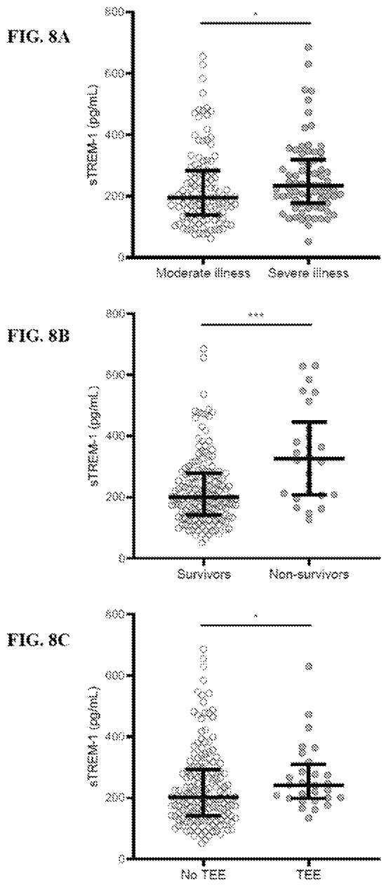

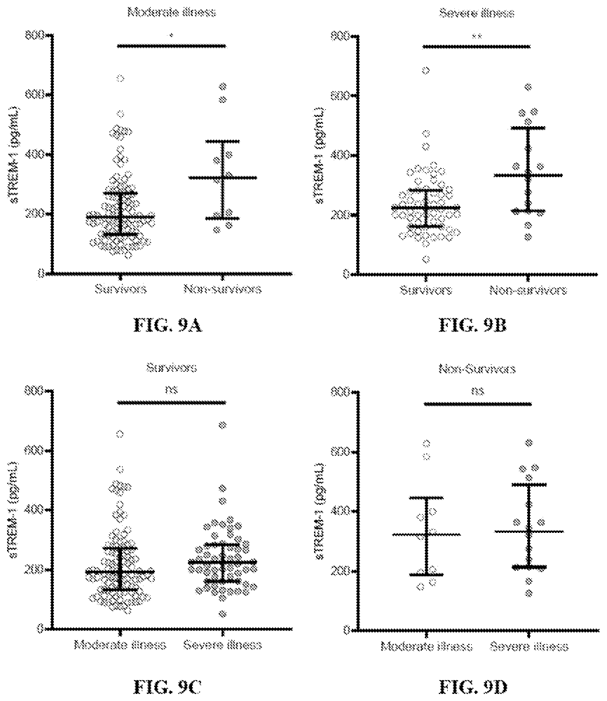

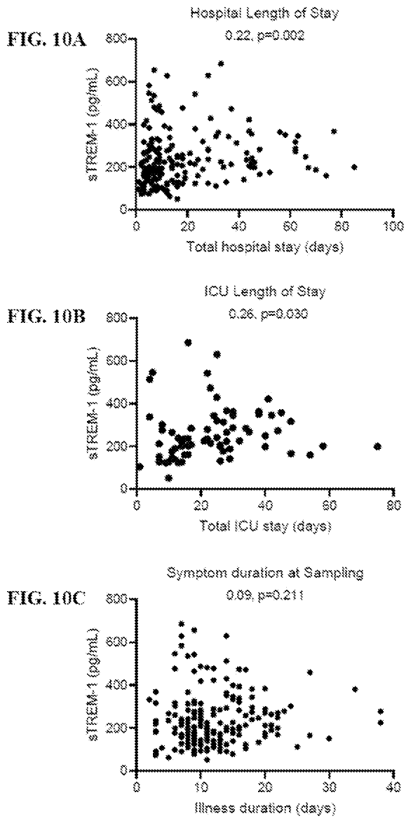

As shown on FIG. 8A, higher sTREM-1 plasma concentrations were observed in severely ill patients as compared to patients with moderate illness (235 pg/mL (IQR 176-319) vs. 195 pg/mL (IQR 139-283), respectively, p=0.017). Similarly, as shown on FIG. 8B, non-survivors had higher sTREM-1 plasma concentrations compared to survivors (326 pg/mL (IQR 207-445) vs. 199 pg/mL (IQR 142-278), respectively, p<0.001).
The relationship between sTREM-1 and disease severity or mortality was separately assessed in these subgroups, indicating the strongest difference in sTREM-1 concentrations in survivors vs. non-survivors (FIGS. 9A-D).
Furthermore, patients who developed thromboembolic events (n=28, 14%) presented with increased sTREM-1 concentrations compared to patients without this complication (FIG. 8C, p=0.044). Thromboembolic events encompassed pulmonary embolisms (n=26), cerebrovascular accidents (n=2) and deep venous thrombosis (n=1). Additionally, sTREM-1 levels were weakly positively correlated with the total duration of hospital stay and ICU stay (r=0.22, p=0.002 and r=0.26, p=0.030, respectively, FIGS. 10A-B), but no correlation was found with duration of symptoms at time of baseline sample collection (r=0.09, p=0.211; FIG. 10C). These latter findings suggest that sTREM-1 may be persistently increased during the disease course in severely ill patients.

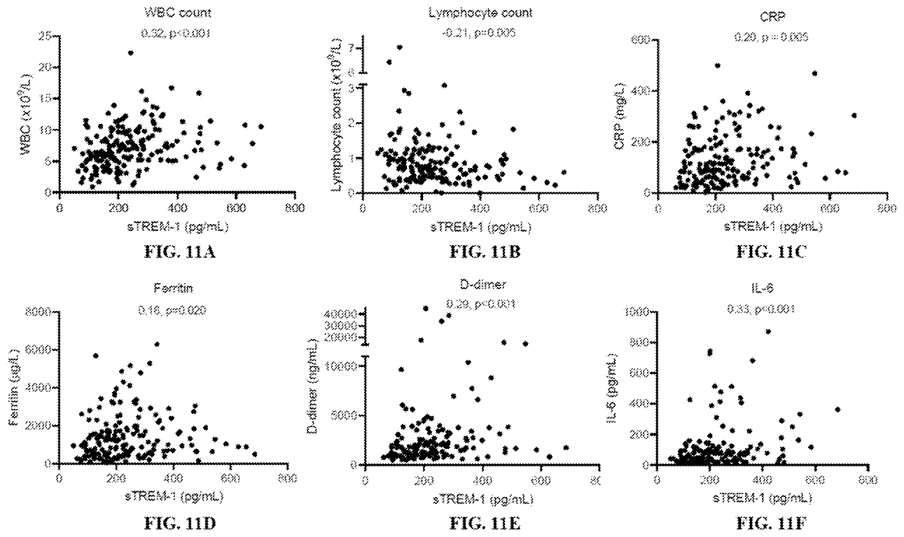

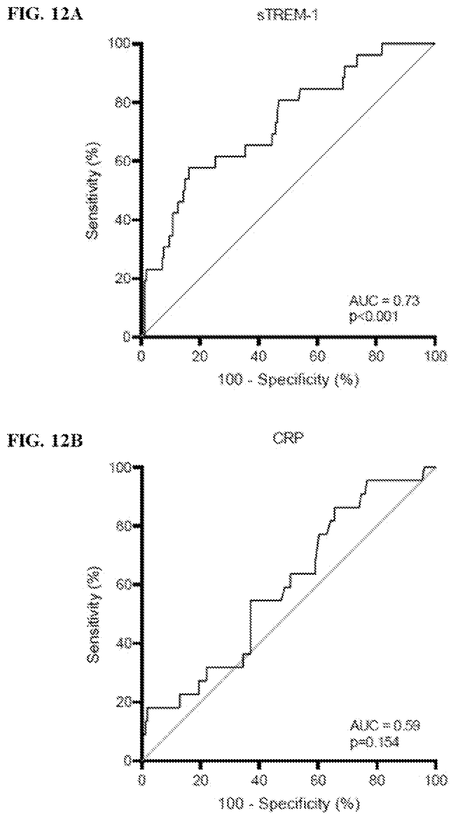

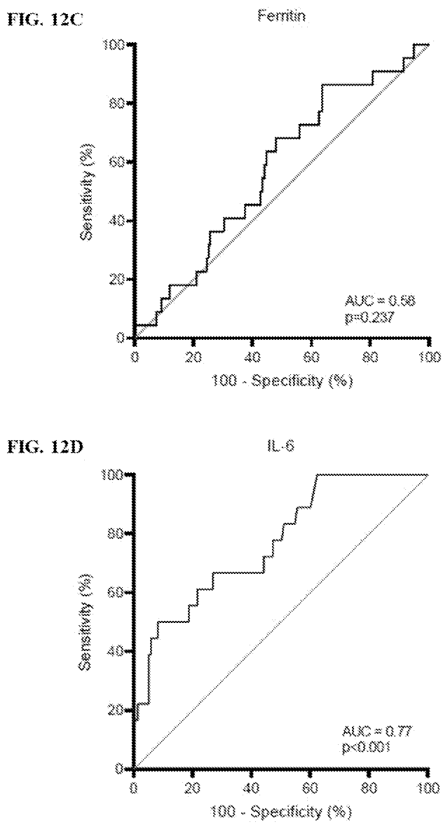

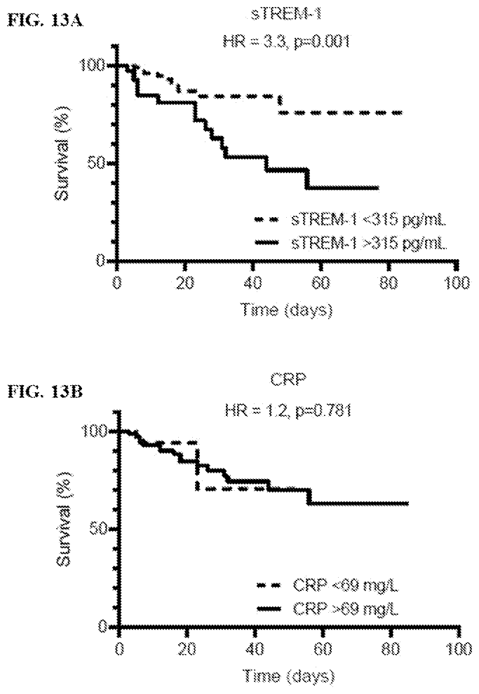

Positive correlations were observed for sTREM-1 concentration and white blood cell (WBC) count, lymphocyte count, C-reactive protein (CRP), ferritin, D-dimer and IL-6 (see FIGS. 11A-F, respectively). These findings indicate that sTREM-1 concentrations are directly associated with a systemic inflammatory response, although they may be independent of standard laboratory inflammatory markers such as CRP or ferritin.

Discriminatory Power of sTREM-1 on Mortality

FIG. 12 presents the ROC-curve for discrimination between survivors and non-survivors based on sTREM-1 concentrations (FIG. 12A); the ROC-curves for CRP (FIG. 12B), ferritin (FIG. 12C) and IL-6 (FIG. 12D) are presented for comparison. The characteristics of the tests conducted for each biomarker are provided in Table 10 below.

TABLE 10
Test characteristics

| | Cut-off | | Youden’s J | Sensitivity | Specificity | PPV | NPV |
|---|---|---|---|---|---|---|---|
| sTREM-1 | >315 | pg/ml | 0.41 | 57.7% | 83.7% | 35.7% | 92.7% |
| CRP | >69 | mg/mL | 0.21 | 86.4% | 34.4% | 17.1% | 94.2% |
| Ferritin | >665 | μg/mL | 0.23 | 86.4% | 36.2% | 17.5% | 94.4% |
| IL-6 | >237 | pg/ml | 0.42 | 50.0% | 91.7% | 48.6% | 92.1% |

AUC: area under the curve.
CRP: C-reactive protein.
IL-6: interleukin-6.
PPV: positive prediction value.
NPV: negative prediction value.

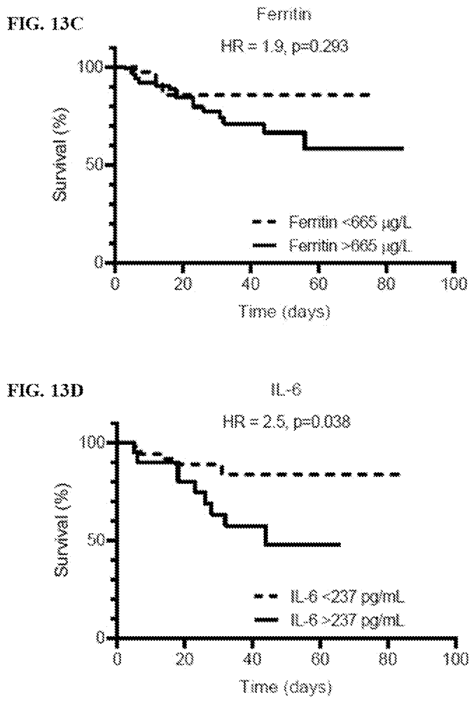

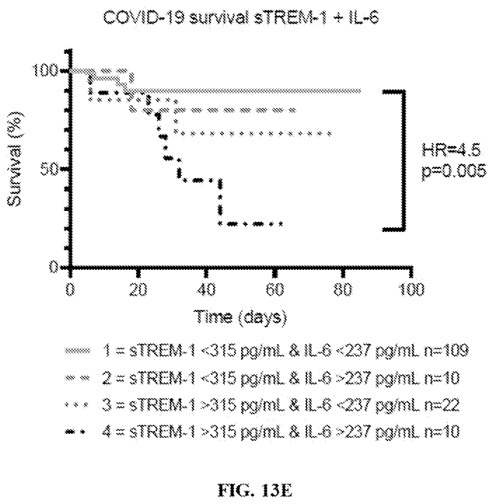

The AUC for sTREM-1 was 0.73 (95% CI 0.62-0.83) indicating moderate discrimination between survivors and non-survivors. The discriminatory power of sTREM-1 was similar to that of IL-6 (AUC=0.77, 95% CI 0.65-0.88; p=0.887), but performed better than that of CRP (AUC=0.59, 95% CI 0.47-0.72; p=0.132) and ferritin (AUC=0.58, 95% CI 0.46-0.70; p=0.078), although not significantly different. Next, survival analysis for sTREM-1 indicated that patients with sTREM-1 plasma concentrations of more than 315 pg/mL had an increased risk for in-hospital mortality (hazard ratio=3.3, 95% CI 1.4-7.8) (FIG. 13A). Hazard ratios of mortality for high circulating concentrations of CRP (FIG. 13B), ferritin (FIG. 13C), and IL-6 (FIG. 13D) were 1.2 (95% CI 0.4-3.7), 1.9 (95% CI 0.7-5.1), and 2.5 (95% CI 0.9-6.9), respectively.

Combining the use of sTREM-1 and IL-6 did not improve the discrimination between survivors and non-survivors. The AUC for the combination of sTREM-1 and IL-6 was 0.79, not statistically different from that of IL-6 alone (p=0.562). Similarly, combining the use of sTREM-1 and IL-6 in a survival analysis (see FIG. 13E) did not show a significant difference as compared to either of the two biomarkers alone. Nevertheless, a significant difference was observed in patients presenting with sTREM-1>315 pg/mL and IL-6>237 pg/mL, compared to patients presenting with sTREM-1<315 pg/mL and IL-6<237 pg/mL (HR: 4.5, p=0.005).

The data presented above in Example 1 and Example 2 demonstrate that sTREM-1 concentrations are significantly increased in patients with COVID-19, and show that they are correlated with severity of the disease and with mortality (see FIGS. 1-2 and 8). Discrimination between patients requiring <15 days under mechanical ventilation and patients >15 days under mechanical ventilation (see FIG. 5), and between survivors and non-survivors was thus possible based on sTREM-1 plasma concentrations (see FIGS. 12A and 13A). Accordingly, patients presenting with high sTREM-1 at entry to hospital or at entry to ICU have a lower likelihood to be extubated before 15 days or to survive (see FIG. 6). The data also demonstrate that elevated sTREM-1 concentrations are significantly correlated with the presence of a complication, such as cardiovascular failure (see FIG. 4B) and thromboembolic events, i.e., thrombotic complications (see FIG. 8C).

### Example 3

### Materials and Methods

sTREM-1 was quantified in 109 plasma samples collected from patients recruited in the study NCT03158948 by ELISA (Human TREM-1 Quantikine® ELISA kit, R&D Systems, reference DTRM10C) and by ECLIA (Elecsys from Roche Diagnostics). The ECLIA method comprises an incubation of the samples with a first sTREM-1-specific antibody which is biotinylated and a second sTREM-1-specific antibody which is labeled with a ruthenium complex to form a sandwich complex. After the addition of streptavidin-coated microparticles, a second incubation allows the binding of the sandwich complex to the microparticles, thus forming a solid phase. The reaction mixture is then aspirated into the measuring cell of an analyzer (Cobas analyzer from Roche Diagnostics), where the microparticles are magnetically captured onto the surface of an electrode. Unbound substances are removed. The application of a voltage to the electrode excites the ruthenium complex and induces a chemiluminescent emission which is measured by a photomultiplier. Results are determined via a calibration curve. The ELISA method comprises a first incubation of the samples in a plate (96-well plate) with wells pre-coated with a capture sTREM-1-specific antibody. After several washes of the wells, the samples are incubated with a detection sTREM-1-specific antibody which is coupled to horseradish peroxidase (HRP). After several washes, a colorimetric reagent is added, followed by a stop solution 30 minutes later. The optical density (OD) is measured in each well within 30 minutes, using a microplate reader set to 450 nm, and wavelength correction with OD 540 nm. Results are determined via a calibration curve

### Results

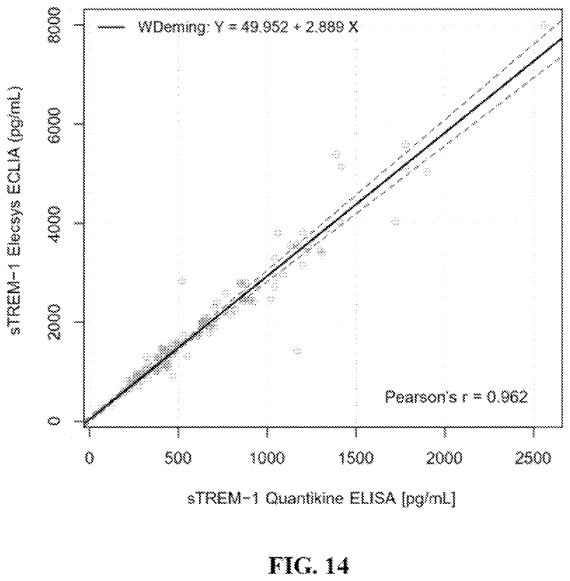

The correlation between the sTREM-1 plasma concentrations measured with the Elecsys sTREM-1 ECLIA assay and those measured with the Quantikine sTREM-1 ELISA assay was investigated. Results show a linear relation between the sTREM-1 plasma concentrations obtained with the two methods. As shown on FIG. 14, the measurements are highly correlated (Pearson's r 0.962, Spearman's rho 0.962, Kendall's tau 0.858). The regression analysis using a weighted Deming regression yields an intercept at 50.0 (−6.41; 106) pg/ml and a slope of 2.89 (2.74; 3.04).

1-10. (canceled)

11. An in vitro method for identifying a subject suffering from a disease caused by a coronavirus infection as being at risk of having or developing a severe form and/or a complication of the disease caused by a coronavirus infection, or at risk of death occurring after the coronavirus infection, said method comprising:
measuring the level of soluble triggering receptor expressed on myeloid cells-1 (sTREM-1) in a biological sample from the subject; and
comparing the level of sTREM-1 measured in the biological sample from the subject to a reference value.

12. A method for treating a subject suffering from a disease caused by a coronavirus identified as being at risk of having or developing a severe form and/or a complication of the disease caused by a coronavirus, or at risk of death occurring after the coronavirus infection, said method comprising:
identifying a subject suffering from a disease caused by a coronavirus as being at risk of having or developing a severe form and/or a complication of the disease caused by a coronavirus, or at risk of death occurring after the coronavirus infection, according to the method of claim 11; and
treating the subject suffering from a disease caused by a coronavirus identified as being at risk of having or developing a severe form and/or a complication of the disease caused by a coronavirus, or at risk of death occurring after the coronavirus infection, by administering to said subject at least one of the following: respiratory support, vasopressor therapy, fluid therapy, antimicrobial therapy, antiviral therapy, cardiovascular support, renal replacement therapy, sedation, an antiviral agent, an anti-interleukin 6 (anti-IL-6) agent, or any mixes thereof.

13. The in vitro method according to claim 11, wherein the complication of the disease caused by a coronavirus infection is selected from the group consisting of respiratory failure, including acute respiratory failure or acute respiratory distress syndrome (ARDS); persistence of respiratory failure including the requirement for prolonged mechanical ventilation, and failed extubation; secondary infection or superinfection; thrombotic complications including venous and/or arterial thromboembolism; pulmonary embolism; cardiocirculatory failure (also referred to as cardiovascular failure); renal failure; liver failure; and any combinations thereof.

14. The in vitro method according to claim 11, wherein the coronavirus is severe acute respiratory syndrome coronavirus 2 (SARS-CoV-2) and the disease caused by a coronavirus infection is coronavirus disease 2019 (COVID-19).

15. The in vitro method according to claim 11, wherein the level of sTREM-1 is a transcription level of sTREM-1 or a translation level of sTREM-1.

16. The in vitro method according to claim 11, wherein the biological sample is a blood sample.

17. The in vitro method according to claim 11, wherein the biological sample is a serum sample.

18. The in vitro method according to claim 11, wherein the subject requires hospitalization.

19. The in vitro method according to claim 18, wherein the subject requires respiratory support.

20. The in vitro method according to claim 18, wherein the level of sTREM-1 is measured at day 3 following hospitalization.

21. An in vitro method for determining the severity of a disease caused by a coronavirus infection in a subject, said method comprising:
measuring the level of soluble triggering receptor expressed on myeloid cells-1 (sTREM-1) in a biological sample from the subject; and
comparing the level of sTREM-1 measured in the biological sample from the subject to a reference value.

22. The in vitro method according to claim 21, wherein the method allows the monitoring over time of the disease caused by a coronavirus infection in the subject, and wherein the method comprises measuring the level of sTREM-1 in biological samples from the subject obtained on at least two occasions.

23. The in vitro method according to claim 22, wherein the at least two occasions are separated by at least 24 hours.

24. The in vitro method according to claim 21, wherein the coronavirus is severe acute respiratory syndrome coronavirus 2 (SARS-CoV-2) and the disease caused by a coronavirus infection is coronavirus disease 2019 (COVID-19).

25. The in vitro method according to claim 21, wherein the level of sTREM-1 is a transcription level of sTREM-1 or a translation level of sTREM-1.

26. The in vitro method according to claim 21, wherein the biological sample is a blood sample.

27. The in vitro method according to claim 21, wherein the biological sample is a serum sample.

28. The in vitro method according to claim 21, wherein the subject requires hospitalization.

29. The in vitro method according to claim 28, wherein the subject requires respiratory support.

30. The in vitro method according to claim 28, wherein the level of sTREM-1 is measured at day 3 following hospitalization.
